(19) United States
(12) Patent Application Publication
(10) Pub. No.: US 2025/0314656 A1
(43) Pub. Date: Oct. 9, 2025

(54) GENETICALLY ENCODED FLUORESCENT INDICATOR OF D-2-HYDROXYGLUTARATE

(71) Applicants: Evan P.S. Pratt, Ishpeming, MI (US); Matthew J. Jennings, Marquette, MI (US)

(72) Inventors: Evan P.S. Pratt, Ishpeming, MI (US); Matthew J. Jennings, Marquette, MI (US)

(21) Appl. No.: 19/083,306

(22) Filed: Mar. 18, 2025

Related U.S. Application Data
(60) Provisional application No. 63/566,728, filed on Mar. 18, 2024.
(60) Provisional application No. 63/739,789, filed on Dec. 30, 2024.

(51) Int. Cl. G01N 33/58; A61K 31/7088; A61K 38/16; C12N 15/62; C12N 15/63; G01N 33/50; G01N 33/68

(52) U.S. Cl. CPC G01N 33/582; A61K 31/7088; A61K 38/16; C12N 15/62; C12N 15/63; G01N 33/5091; G01N 33/68

(57) ABSTRACT

Constructs for detection of d-2-hydroxyglutarate (d-2-HG), and their use in determining the IDH1/2 mutational status of a biological sample obtained from a subject, monitoring a change in D-2-HG levels in a subject, and analysing D-2-HG in a biological sample obtained from a subject, comprising detecting for D-2-HG in the sample or subcellular compartment therein, and methods for the same. Nucleic acid molecules, vectors, cells, and pharmaceutical compositions are also described.

### CROSS REFERENCE TO RELATED APPLICATIONS

This utility application claims priority of U.S. Provisional Patent Application No. 63,566,728, filed Mar. 18, 2024, and U.S. Provisional Patent Application No. 63/739,789, filed Dec. 30, 2024, both co-pending herewith, the disclosures of which are incorporated herein by reference in their entirety.

### INCORPORATION BY REFERENCE

This application incorporates by reference the sequence listing and information submitted simultaneously herewith in the XML file titled 065483-00183_Sequence_Listing_10043704-1, having a production date of 2025 Mar. 18 and a file size of 43kb.

### FIELD OF THE INVENTION

The invention relates to a genetically encoded fluorescent biosensor for detecting D-2-hydroxyglutarate (D-2-HG), and more particularly to a fluorescent biosensor using a transcription factor DhdR as a D-2-HG-sensing domain wherein a unique set of fluorescent reporters provide a superior dynamic range in detecting D-2-HG.

### BACKGROUND OF THE INVENTION

D-2-hydroxyglutarate (D-2-HG) is an oncometabolite that is associated with several forms of cancer, and most notably glioma (Dang et al., 2009) and acute myeloid leukemia (AML) (Raimondi et al., 2022). Cancers that produce elevated levels of D-2-HG harbor mutations in the active site of the enzymes isocitrate dehydrogenase 1 and 2 (IDH1/2). Wild-type IDH1/2 converts isocitrate to α-ketoglutarate, reducing NADP⁺ to NADPH; however, mutations at R132 in IDH1 and R140 or R172 in IDH2 result in neomorphic activity (Reitman & Yan, 2010). IDH1/2 mutant enzymes catalyze the reduction of α-ketoglutarate to D-2-HG and have been shown to be an early event in the development of cancer. The accumulation of D-2-HG promotes oncogenesis through the competitive inhibition of enzymes belonging to the of α-ketoglutarate/Fe(II)-dependent dioxygenase family (Ye et al., 2018). This leads to epigenetic changes associated with the Global-CpG Island Methylation Phenotype (G-CIMP) (Turcan et al., 2012) and aberrant expression of oncogenes and tumor suppressor genes. Other deleterious effects of D-2-HG include altered expression of hypoxia-inducible factor 1-alpha (HIF1α), metabolic reprogramming (Miller et al., 2023), and the accumulation of lipid reactive oxygen species (ROS) (Wang et al., 2019). Cells associated with G-CIMP are thought to be less aggressive in part due to the delayed repair of double stranded DNA breaks. Resultantly, IDH1/2 mutant cells are highly sensitive to DNA damaging agents such as temozolomide or radiation and are more susceptible to poly (ADP)-ribose polymerase (PARP) inhibitors. In general, IDH mutations are associated with a better patient outcome, as they render cells more vulnerable to death and demonstrate reduced levels of migration, angiogenesis, and invasion; thus, a mutant version of this enzyme is a significant and positive prognostic biomarker. In light of the importance of D-2-HG in cancer, robust tools for detection and characterization are essential. While the epigenetic and genetic alterations associated with elevated D-2-HG occur in the nucleus, IDH1 and IDH2 are localized to the cytosol and mitochondria, respectively. Furthermore, D-2-HG is catabolized by D-2-HG dehydrogenase in the mitochondrial matrix (Achouri et al., 2004). Collectively, this infers the presence of D-2-HG in at least three subcellular locations. However, the spatial distribution of D-2-HG has not been investigated. Therefore, a tool for studying D-2-HG spatiotemporal interactions at the subcellular level is warranted.

Given the considerable impact of IDH mutations on cancer phenotype and patient prognosis, efficient and timely identification of this mutation is critical. Due to the lengthy analytical times of currently available diagnostic methods such as sequencing and immunohistochemistry (IHC), clinicians do not have knowledge of the tumor's genetic characteristics until days or weeks post-surgery. As an alternative to the direct detection of IDH mutations, the detection of D-2-HG may be used as a surrogate marker for the presence of IDH1/2 mutations. D-2-HG can be detected by liquid chromatography-mass spectroscopy (LC-MS) (Fujita et al., 2022), (Tuna et al., 2022), (Struys et al., 2004), (Zhang et al., 2023), gas chromatography-mass spectroscopy (GC-MS) (Fernández-Galán et al., 2018) or magnetic resonance spectroscopy (MRS) (Choi et al., 2012). These methods are technically complex, lack ideal sensitivity, and cannot provide real-time quantification. While these methods are well characterized, they are not amenable for rapid and routine use in a clinical laboratory.

Furthermore, these methods cannot readily distinguish between D-2-HG and its enantiomer L-2-HG. Additionally, the recent advent of mutant IDH1 inhibitors makes real-time monitoring of D-2-HG levels in patients imperative as its depletion can be utilized to gauge the effectiveness of inhibitor therapy (Mellinghoff et al., 2023).

Studies which have explored the relationship between D-2-HG level and disease largely disagree on optimal sample type and the relative quantity of oncometabolite corresponding to IDH mutational status (Table 1). This lack of consensus warrants a standardized method for the noninvasive, rapid quantification of D-2-HG in body fluids to allow for the preoperative, intraoperative, and/or postoperative detection of an IDH mutation.

TABLE 1
Summary of D-2-HG or L-2-HG concentrations in biological matrices
of glioma patients as presented in current literature.

| Enan- | Tissue | IDH Mutant | IDH Wildtype | |
|---|---|---|---|---|
| tiomer | Type | (μM) | (μM) | Reference |
| D | Blood | 0.81 (0.51-1.32) | 0.75 (0.41-1.70) | Delahousse et al., 2018, |
| | | 0.37 ± 0.13 | — | Lee et al., 2024, |
| | | 10.9 | 0.8 (0.7-0.9) | Tuna et al., 2022, |
| | | 2.94 | 1.61 | Strain et al., 2021. |
| D | CSF | 0.15 (0.05-11.4) | 0.08 (0.05-0.5) | Kalinina et al., 2016, |
| | | 7.4 (0.3-109.0) | 0.4 (0.1-2.6) | Fujita et al., 2022, |
| | | 3.3 ± 0.5 | 2.4 ± 0.3 | Tuna et al., 2022 |
| D | Tissue | 1964.8 (median) | 14.0 (median) | Sim et al., 2019 |
| L | Blood | 0.58 (0.36-1.16) | 0.80 (0.37-1.50) | Lee et al., 2024, |
| | | 0.41 ± 0.26 | — | Tuna et al., 2022, |
| | | 1.24 | 1.32 | Strain et al., 2021 |
| L | CSF | 0.35 (0.01-0.5) | 0.35 (0.1-0.88) | Kalinina et al., 2016, |
| | | 0.9 (0.4-4.1) | 1.0 (0.1-6.1) | Fujita et al., 2022, |
| | | 2.5 ± 0.3 | 2.6 ± 0.23 | Tuna et al., 2022 |
| Total 2HG | Blood | 1.6 ± 0.4 | 1.29 ± 0.13 | Lee et al., 2024, |
| | | 0.66 ± 0.41 | 0.65 ± 0.30 | Lombardi et al., 2015, |
| | | 0.89 (0.22-1.91) | 0.85 (0.24-1.87) | Fathi et al., 2016, |
| | | 0.79 ± 0.36 | — | Capper et al., 2012 |
| Total 2HG | CSF | 5.9 ± 0.6 | 5.0 ± 0.5 | Fujita et al., 2022 |
| Total 2HG | Urine | 20.0 (7.0-136.9) | 11.6 (5.6-37.5) | Fathi et al., 2016 |
| Total 2HG | Tissue | 1971.5 (median) | 27.0 (median) | Sim et al., 2019 |

### SUMMARY OF THE INVENTION

D-2-HG is an oncometabolite that accumulates in response to certain mutations in isocitrate dehydrogenase 1 or 2 (IDH1/2). While D-2-HG is often used as a surrogate marker for IDH1/2 mutant cancers, simple and enantiomer-specific detection methods are limited. In this study, we present a first-generation genetically encoded fluorescent sensor that is highly specific for D-2-HG compared to other structurally similar metabolites and demonstrates a greater affinity for D-2-HG over L-2-HG. D2HGlo robustly quantifies D-2-HG in a variety of biological fluids and accurately predicted the IDH mutational status of archived glioma tumor supernatants. The reportable range of detection for D2HGlo suggests that it may be a powerful tool for detecting elevated levels of D-2-HG, measuring the efficacy of pharmaceutical inhibitors, and monitoring remission vs. recurrence in patients with IDH mutant cancers. This sensor also facilitated preliminary investigations of the intracellular distribution of D-2-HG in living human cells to include its presence in the nuclear compartment. D2HGlo was used to perform a side-by-side comparison of cytosolic and secreted D-2-HG to reveal that glycolysis, but not glutamine catabolism, drives D-2-HG production in IDH1 mutant cells.

The inventors of the present patent application have developed a genetically encoded fluorescent sensor of D-2-HG, which is termed “D2HGlo”, that rapidly detects and quantifies D-2-HG in biological samples, including cell culture media, artificial cerebrospinal fluid, serum, and urine. The D2HGlo sensor, or in short, D2HGlo, directly analyzed supernatants from tumor lysates, predicting IDH1 mutational status in gliomas with 100% accuracy. D2HGlo responds to clinically relevant concentrations of D-2-HG, demonstrates exceptional selectivity and can quantify D-2-HG in various body fluids and glioma tumor supernatants. Thus, D2HGlo may be amenable to preoperative or intraoperative detection of IDH1/2 mutations and postoperative monitoring of D-2-HG levels in patients treated with mutant IDH1 inhibitors. In addition to D2HGlo's clinical utility, the present application also presents preliminary findings for its adaptation to the cellular environment. To assess D-2-HG production in living immortalized glioma cells, the inventive D2HGlo sensors have been engineered that localize to subcellular compartments. D2HGlo performs robustly in situ, where it demonstrates the specific detection of endogenous and exogenous D-2-HG levels at the subcellular level. As no tool currently exists to study the spatial distribution of D-2-HG in living cells, this patent application presents novel findings of elevated levels within the nucleus, mitochondria, and cytoplasm of IDH1 mutant cells. These results offer valuable insight into the oncogenic mechanisms driving IDH1/2 mutant cancers and may aid in further elucidation of its role in disease.

Thus, in this patent application and the study disclosed herein, a novel genetically encoded fluorescent sensor (D2HGlo) is presented that is capable of assessing D-2-HG directly in vitro and in situ. This application shows that D2HGlo is highly specific for D-2-HG compared to L-2-HG and other structurally similar metabolites. Additionally, D2HGlo allows visualization of the intracellular distribution and regulation of D-2-HG in living human cells for the first time. Importantly, D2HGlo's range of detection makes it a powerful tool for measuring clinically relevant levels of D-2-HG in biological fluids and tumor samples.

Two other D-2-HG reporters have been recently developed that are based on using the transcription factor DhdR as a D-2-HG-sensing domain. The first biosensor leverages AlphaScreen technology to produce a luminescent signal in the presence of D-2-HG (Xiao et al. 2021, Nature communications, 12(1), 7108). This sensing platform is unlike the one being disclosed herein. A second genetically encoded fluorescent biosensor called DHGFR1.0 was also published (Xiao et al. 2023, Sensors and Actuators, 385, 133681).

Compared with DHGFR1.0, the sensor being disclosed in the current application, D2HGlo, contains a truncated version of DhdR that drastically increases sensor response. In addition, the inventive D2HGlo contains a different set of fluorescent reporters than DHGFR1.0 that result in blue and yellow light being emitted, as opposed to green and red light. The main advantages of D2HGlo over DHGFR1.0 are its far superior dynamic range, its proven clinical and research utility in detecting D-2-HG in complex mediums and that D2HGlo functions over a physiologically relevant pH range. DHGFR1.0 only responds to D-2-HG at pH 10, which is physiologically improbable.

D2HGlo is a genetically encoded fluorescent biosensor of D-2-hydroxyglutarate (D-2-HG). D-2-HG is produced in IDH1/2 mutant cancers, including gliomas. D2HGlo contains a D-2-HG-sensing domain (derived from the bacterial transcription factor DhdR) and two fluorescent proteins (ECFP and cpVenus 173) that serve as the reporting elements. The DNA sequence of D2HGlo is encoded by two different expression vectors: pcDNA3 and pBAD. pcDNA3 that encodes for D2HGlo can be transfected into mammalian cells for in situ detection of D-2-HG. pBAD that encodes for D2Hglo can be transformed into competent bacteria. The D2HGlo polypeptide can be subsequently isolated and purified for in vitro detection of D-2-HG.

pBAD encoding D2HGlo are transformed into competent E. Coli, which are grown up in lysogeny broth for 3-4 hours. D2HGlo overexpression is induced with the addition of 0.2% L-arabinose. Following 24 hours of protein expression at room temperature, the bacteria are lysed using B-PER supplemented with lysozyme and DNAase-I. D2HGlo can be purified using a Cobalt Metal Affinity Resin and desalted using a PD-10 desalting column.

Purified D2HGlo can be used to detect D-2-HG in biological fluids, including serum, plasma, urine and cerebrospinal fluid, and may be useful in monitoring D-2-HG in patients with cancers that cause an increase in D-2-HG. The purified sensor can be mixed with biological fluids in a 96-well plate format and the fluorescence readout can be measured using a standard microplate reader. For characterization of D-2-HG-producing cancers, there is the potential for D2HGlo to be used preoperatively, intraoperatively and/or postoperatively to detect the presence of D-2-HG in patient samples. To compliment the clinical utility of D2HGlo, D2HGlo can also be used in basic science research labs to examine D-2-HG distribution, dynamics and regulation in living mammalian cells using a standard fluorescence microscope.

The Applicant has synthesised constructs which are able to bind and detect D-2-HG with high specificity. Such constructs may detect and quantify D-2-HG directly in vitro and in situ, allowing analysis of the intracellular distribution and regulation of D-2-HG.

According to the invention there is provided a construct (such as a fusion protein) for detection of d-2-hydroxyglutarate (d-2-HG), comprising:
a first fluorescent reporter;
a second fluorescent reporter; and
a DhdR transcription factor, or a variant thereof;
wherein the first fluorescent reporter is linked at the N-terminus of the DhdR transcription factor, or variant thereof, and the second fluorescent reporter is linked at the C-terminus of the DhdR transcription factor, or variant thereof, or wherein the first fluorescent reporter is linked at the C-terminus of the DhdR transcription factor, or variant thereof, and the second fluorescent reporter is linked at the N-terminus of the DhdR transcription factor, or variant thereof.

The variant of the DhdR transcription factor is preferably a truncated version of the DhdR transcription factor, as described herein.

### Reporters

The first reporter and second reporter may be such that one is an energy donor and one is an energy acceptor. For example, the first reporter may act as an energy donor and the second reporter may act as an energy acceptor. Energy may thus be transferred from the donor to the acceptor. The donor may emit light which excites the acceptor, causing the acceptor to emit light at a different wavelength to the donor.

The first reporter is preferably a fluorescent reporter. The fluorescent reporter may be excited by light. The second reporter is preferably a fluorescent reporter. The second fluorescent reporter may be excited by light. The first and second fluorescent reporters may form a Fluorescence Resonance Energy Transfer (FRET) pair.

Optionally, the first fluorescent reporter has an emission peak which is less than 490 nm. Optionally, the first fluorescent reporter has an emission peak which is less than 480 nm. Preferably, the first fluorescent reporter has an emission peak which is about 477 nm.

Optionally, the first fluorescent reporter has an emission peak which is more than 450 nm. Optionally, the first fluorescent reporter has an emission peak which is more than 460 nm. Optionally, the first fluorescent reporter has an emission peak which is more than 470 nm

Optionally, the first fluorescent reporter has an excitation peak which is less than 480 nm. Optionally, the first fluorescent reporter has an excitation peak which is less than 470 nm, 460 nm, 450 nm, or 440 nm. Preferably, the first fluorescent reporter has an excitation peak which is about 434 nm.

Optionally, the first fluorescent reporter has an excitation peak which is more than 400 nm. Optionally, the first fluorescent reporter has an excitation peak which is more than 410 nm, 420 nm, or 430 nm.

Optionally, the second fluorescent reporter has an excitation peak which is less than 550 nm. Optionally, the second fluorescent reporter has an excitation peak which is less than 540 nm, 530 nm, 520 nm, or 510 nm. Preferably, the second fluorescent reporter has an excitation peak which is about 500 nm.

Optionally, the second fluorescent reporter has an excitation peak which is more than 450 nm. Optionally, the second fluorescent reporter has an excitation peak which is more than 460 nm, 470 nm, 480 nm, or 490 nm.

Optionally, the second fluorescent reporter has an emission peak which is more than 490 nm. Optionally, the second fluorescent reporter has an emission peak which is more than 500 nm, or 510 nm. Preferably, the second fluorescent reporter has an emission peak which is about 520 nm.

Optionally, the second fluorescent reporter has an emission peak which is less than 550 nm. Optionally, the second fluorescent reporter has an emission peak which is less than 540 nm, or 530 nm.

Optionally, the first and the second fluorescent reporter comprise first and second fluorescent proteins, respectively.

Optionally, the first fluorescent protein is enhanced cyan fluorescent protein (ECFP), and the second fluorescent protein is cpVenus173.

Optionally, the first fluorescent protein is cpVenus173, and the second fluorescent protein is ECFP.

Optionally, the amino acid sequence of ECFP comprises amino acid sequence of SEQ ID NO:6.

Optionally, the amino acid sequence of cpVenus173 comprises amino acid sequence of SEQ ID NO: 8.

Alternatively, the first and second fluorescent reporters may be selected form Clover, mRuby2, mTFP and Venus.

### Linkers

Optionally, the first reporter is linked at the N-terminus of the DhdR transcription factor, or variant thereof, and the second reporter is linked at the C-terminus of the DhdR transcription factor, or variant thereof.

Optionally, the first reporter is linked at the C-terminus of the DhdR transcription factor, or variant thereof, and the second reporter is linked at the N-terminus of the DhdR transcription factor, or variant thereof.

Optionally, the first reporter is linked at the N-terminus of the DhdR transcription factor, or variant thereof, by a first linker (which may also be termed a first spacer), and the second reporter is linked at the C-terminus of the DhdR transcription factor, or variant thereof, by a second linker (which may also be termed a second spacer).

The first and/or second linker preferably comprises one of more amino acids. The first and/or second linker may be a peptide.

The one or more amino acids in the first and/or second linker may thus be distinct from the amino acids of the DhdR transcription factor. So, for example, one or more amino acids may be joined to the N-terminus of the DhdR transcription factor to form the linker, and one or more amino acids may be joined to the C-terminus of the DhdR transcription factor to form the linker. The first and/or second linker may thus comprise amino acid residues which do not occur naturally at the N and/or C-terminus of the DhdR transcription factor.

The one or more amino acids of the first and/or second linker may thus be distinct from the amino acids of the first and/or second reporters, if the first and/or second reporters are proteins (e.g. fluorescent proteins). So, for example, one or more amino acids may be joined to the N-terminus or C-terminus of the first reporter to form the linker, and one or more amino acids may be joined to the N-terminus or C-terminus of the second reporter to form the linker. The first and/or second linker may thus comprise amino acid residues which do not occur naturally at the N and/or C-terminus of the first and/or second reporters.

Optionally, the first reporter is linked at the N-terminus of the DhdR transcription factor, or variant thereof, by a first linker which comprises at least one amino acid residue, and the second reporter is linked at the C-terminus of the DhdR transcription factor, or variant thereof, by a second linker which comprises at least one amino acid residue.

Optionally, the first reporter is linked at the C-terminus of the DhdR transcription factor, or variant thereof, by a first linker which comprises at least one amino acid residue, and the second reporter is linked at the N-terminus of the DhdR transcription factor, or variant thereof, by a second linker which comprises at least one amino acid residue.

Optionally, the first linker comprises one to ten amino acid residues.

Optionally, the second linker comprises one to ten amino acid residues.

Optionally, the first linker comprises one to five amino acid residues.

Optionally, the second linker comprises one to five amino acid residues.

Optionally, the first linker comprises two amino acid residues.

Optionally, the second linker comprises two amino acid residues.

Optionally, the first linker comprises three amino acid residues.

Optionally, the second linker comprises three amino acid residues.

Optionally, the first linker comprises four amino acid residues.

Optionally, the second linker comprises four amino acid residues.

Optionally, the first linker comprises five amino acid residues.

Optionally, the second linker comprises five amino acid residues.

Optionally, the first linker comprises an amino acid R.

Optionally, the first linker comprises an amino acid sequence of R and M.

Optionally, the first linker comprises an amino acid sequence of R, M, and H.

Optionally, the second linker comprises an amino acid R.

Optionally, the second linker comprises an amino acid sequence of R and M.

Optionally, the second linker comprises an amino acid sequence of R, M, and H.

Optionally, the second linker comprises an amino acid E.

Optionally, the second linker comprises an amino acid sequence of E, and L.

Optionally, the first linker comprises an amino acid E.

Optionally, the first linker comprises an amino acid sequence of E, and L.

Preferably, the first linker comprises (or consists of) an amino acid sequence of R, M, and H, and the second linker comprises (or consists of) an amino acid sequence of E, and L.

Preferably, the first linker comprises (or consists of) an amino acid sequence of E, and L, and the second linker comprises (or consists of) an amino acid sequence of R, M, and H.

### DhdR Transcription Factor, or Variant Thereof

Optionally, the DhdR transcription factor is a full-length or wild-type protein. For example, it may comprises an amino acid sequence of SEQ ID NO:1, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:1.

Preferably, the variant of the DhdR transcription factor comprises a truncated DhdR transcription factor for binding d-2-HG. For example, amino acids may be truncated (or removed) from the N-terminus and/or the C-terminus of the DhdR transcription factor. Preferably, amino acids are truncated from both the N-terminus and C-terminus.

Optionally, up to 10 amino acid residues are truncated from the N-terminus of DhdR corresponding to the terminal N-terminus amino acid residues of DhdR, and/or up to 10 amino acid residues are truncated from the C-terminus of DhdR corresponding to the terminal C-terminus amino acid residues of DhdR.

Optionally, up to 5 amino acid residues are truncated from the N-terminus of DhdR corresponding to the terminal N-terminus amino acid residues of DhdR, and/or up to 5 amino acid residues are truncated from the C-terminus of DhdR corresponding to the terminal C-terminus amino acid residues of DhdR.

Optionally, one amino acid residue is truncated from the N-terminus of the truncated DhdR transcription factor, corresponding to the terminal N-terminus amino acid residue of the DhdR transcription factor.

Optionally, one amino acid residue is truncated from the C-terminus of the truncated DhdR transcription factor, corresponding to the terminal C-terminus amino acid residue of the DhdR transcription factor.

Most preferably, two amino acid residues are truncated from the N-terminus of the truncated DhdR transcription factor, corresponding to the two terminal N-terminus amino acid residues of DhdR transcription factor.

Most preferably, two amino acid residues are truncated from the C-terminus of the truncated DhdR transcription factor, corresponding to the two terminal C-terminus amino acid residues of DhdR transcription factor.

Optionally, three amino acid residues are truncated from the N-terminus of the truncated DhdR transcription factor, corresponding to the three terminal N-terminus amino acid residues of DhdR transcription factor.

Optionally, three amino acid residues are truncated from the C-terminus of the truncated DhdR transcription factor, corresponding to the three terminal C-terminus amino acid residues of DhdR transcription factor.

Optionally, four amino acid residues are truncated from the N-terminus of the truncated DhdR transcription factor, corresponding to the four terminal N-terminus amino acid residues of DhdR transcription factor.

Optionally, four amino acid residues are truncated from the C-terminus of the truncated DhdR transcription factor, corresponding to the four terminal C-terminus amino acid residues of DhdR transcription factor.

Optionally, five amino acid residues are truncated from the N-terminus of the truncated DhdR transcription factor, corresponding to the five terminal N-terminus amino acid residues of DhdR transcription factor.

Optionally, five amino acid residues are truncated from the C-terminus of the truncated DhdR transcription factor, corresponding to the five terminal C-terminus amino acid residues of DhdR transcription factor.

Optionally, one amino acid residue is truncated from the N-terminus of the truncated DhdR transcription factor, corresponding to the terminal N-terminus amino acid residue of DhdR transcription factor, and one amino acid residue is truncated from the C-terminus of the truncated DhdR transcription factor, corresponding to the terminal C-terminus amino acid residue of DhdR transcription factor.

Most preferably, two amino acid residues are truncated from the N-terminus of the truncated DhdR transcription factor, corresponding to the two terminal N-terminus amino acid residues of DhdR, and two amino acid residues are truncated from the C-terminus of the truncated DhdR transcription factor, corresponding to the two terminal C-terminus amino acid residues of DhdR transcription factor.

Optionally, three amino acid residues are truncated from the N-terminus of the truncated DhdR transcription factor, corresponding to the three terminal N-terminus amino acid residues of DhdR transcription factor, and three amino acid residues are truncated from the C-terminus of the truncated DhdR transcription factor, corresponding to the three terminal C-terminus amino acid residues of DhdR transcription factor.

Most preferably, the N-terminus amino acid residues of the truncated DhdR transcription factor are the amino acid residues of SEQ ID NO:18, wherein the amino acid residues are present in the order recited in SEQ ID NO:18, beginning at the terminal N-terminus amino acid residue, or an amino acid sequence that has at least 75% amino acid identity along its entire length with the sequence of SEQ ID NO:18.

Most preferably, the C-terminus amino acid residues of the truncated DhdR transcription factor are the amino acid residues of SEQ ID NO:19, wherein the amino acid residues are present in the order recited in SEQ ID NO:19, beginning at the terminal C-terminus amino acid residue, or an amino acid sequence that has at least 75% amino acid identity along its entire length with the sequence of SEQ ID NO:19.

Optionally, the N-terminus amino acid residues of the truncated DhdR transcription factor are the amino acid residues of SEQ ID NO:20, wherein the amino acid residues are present in the order recited in SEQ ID NO:20, beginning at the terminal N-terminus amino acid residue, or an amino acid sequence that has at least 75% amino acid identity along its entire length with the sequence of SEQ ID NO:20.

Optionally, the C-terminus amino acid residues of the truncated DhdR transcription factor are the amino acid residues of SEQ ID NO:21, wherein the amino acid residues are present in the order recited in SEQ ID NO:21, beginning at the terminal C-terminus amino acid residue, or an amino acid sequence that has at least 75% amino acid identity along its entire length with the sequence of SEQ ID NO:21.

Optionally, the N-terminus amino acid residues of the truncated DhdR transcription factor are the amino acid residues of SEQ ID NO:22, wherein the amino acid residues are present in the order recited in SEQ ID NO:22, beginning at the terminal N-terminus amino acid residue, or an amino acid sequence that has at least 75% amino acid identity along its entire length with the sequence of SEQ ID NO:22.

Optionally, the C-terminus amino acid residues of the truncated DhdR transcription factor are the amino acid residues of SEQ ID NO:23, wherein the amino acid residues are present in the order recited in SEQ ID NO:23, beginning at the terminal C-terminus amino acid residue, or an amino acid sequence that has at least 75% amino acid identity along its entire length with the sequence of SEQ ID NO:23.

Preferably, the truncated DhdR transcription factor comprises an amino acid sequence of SEQ ID NO: 2 (Variant 2 truncated DhdR). Optionally, the truncated DhdR transcription factor comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO: 2.

Optionally, the truncated DhdR transcription factor comprises an amino acid sequence of SEQ ID NO: 14 (Variant 1 truncated DhdR). Optionally, the truncated DhdR transcription factor comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO: 14.

Optionally, the truncated DhdR transcription factor comprises an amino acid sequence of SEQ ID NO: 15 (Variant 3 truncated DhdR). Optionally, the truncated DhdR transcription factor comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO: 15.

Optionally, the variant of DhdR transcription factor comprises an amino acid sequence of SEQ ID NO: 1 in which the amino acid sequence of SEQ ID NO:1 comprises a truncation. Optionally, up to 5 amino acid residues are truncated from the N-terminus of the amino acid sequence of SEQ ID NO:1 corresponding to the terminal N-terminus amino acid residues of the amino acid sequence of SEQ ID NO:1, and/or up to 5 amino acid residues are truncated from the C-terminus of the amino acid sequence of SEQ ID NO:1 corresponding to the terminal C-terminus amino acid residues of the amino acid sequence of SEQ ID NO:1.

Optionally, one amino acid residue is truncated from the N-terminus of the variant of DhdR transcription factor, corresponding to the terminal N-terminus amino acid residue of the amino acid sequence of SEQ ID NO:1.

Optionally, one amino acid residue is truncated from the C-terminus of the variant of DhdR transcription factor, corresponding to the terminal C-terminus amino acid residue of the amino acid sequence of SEQ ID NO:1.

Most preferably, two amino acid residues are truncated from the N-terminus of the variant of DhdR transcription factor, corresponding to the two terminal N-terminus amino acid residues of the amino acid sequence of SEQ ID NO:1.

Most preferably, two amino acid residues are truncated from the C-terminus of the variant of DhdR transcription factor, corresponding to the two terminal C-terminus amino acid residues of the amino acid sequence of SEQ ID NO:1.

Optionally, three amino acid residues are truncated from the N-terminus of the variant of DhdR transcription factor, corresponding to the three terminal N-terminus amino acid residues of the amino acid sequence of SEQ ID NO:1.

Optionally, three amino acid residues are truncated from the C-terminus of the variant of the DhdR transcription factor, corresponding to the three terminal C-terminus amino acid residues of the amino acid sequence of SEQ ID NO:1.

Optionally, four amino acid residues are truncated from the N-terminus of the variant of the DhdR transcription factor, corresponding to the four terminal N-terminus amino acid residues of the amino acid sequence of SEQ ID NO:1.

Optionally, four amino acid residues are truncated from the C-terminus of the variant of the DhdR transcription factor, corresponding to the four terminal C-terminus amino acid residues of the amino acid sequence of SEQ ID NO:1.

Optionally, five amino acid residues are truncated from the N-terminus of the variant of the DhdR transcription factor, corresponding to the five terminal N-terminus amino acid residues of DhdR transcription factor.

Optionally, five amino acid residues are truncated from the C-terminus of the variant of the DhdR transcription factor, corresponding to the five terminal C-terminus amino acid residues of the amino acid sequence of SEQ ID NO:1.

Optionally, one amino acid residue is truncated from the N-terminus of the variant of the DhdR transcription factor, corresponding to the terminal N-terminus amino acid residue of DhdR transcription factor, and one amino acid residue is truncated from the C-terminus of the variant of the DhdR transcription factor, corresponding to the terminal C-terminus amino acid residue of the amino acid sequence of SEQ ID NO:1.

Most preferably, two amino acid residues are truncated from the N-terminus of the variant of the DhdR transcription factor, corresponding to the two terminal N-terminus amino acid residues of DhdR, and two amino acid residues are truncated from the C-terminus of the variant of the DhdR transcription factor, corresponding to the two terminal C-terminus amino acid residues of the amino acid sequence of SEQ ID NO:1.

Optionally, three amino acid residues are truncated from the N-terminus of the variant of the DhdR transcription factor, corresponding to the three terminal N-terminus amino acid residues of DhdR transcription factor, and three amino acid residues are truncated from the C-terminus of the DhdR transcription factor, corresponding to the three terminal C-terminus amino acid residues of the amino acid sequence of SEQ ID NO:1.

Most preferably, the N-terminus amino acid residues of the variant of the DhdR transcription factor are the amino acid residues of SEQ ID NO:18, wherein the amino acid residues are present in the order recited in SEQ ID NO:18, beginning at the terminal N-terminus amino acid residue, or an amino acid sequence that has at least 75% amino acid identity along its entire length with the sequence of SEQ ID NO:18.

Most preferably, the C-terminus amino acid residues of the variant of the DhdR transcription factor are the amino acid residues of SEQ ID NO:19, wherein the amino acid residues are present in the order recited in SEQ ID NO:19, beginning at the terminal C-terminus amino acid residue, or an amino acid sequence that has at least 75% amino acid identity along its entire length with the sequence of SEQ ID NO:19.

Optionally, the N-terminus amino acid residues of the variant of the DhdR transcription factor are the amino acid residues of SEQ ID NO:20, wherein the amino acid residues are present in the order recited in SEQ ID NO:20, beginning at the terminal N-terminus amino acid residue, or an amino acid sequence that has at least 75% amino acid identity along its entire length with the sequence of SEQ ID NO:20.

Optionally the C-terminus amino acid residues of the variant of the DhdR transcription factor are the amino acid residues of SEQ ID NO:21, wherein the amino acid residues are present in the order recited in SEQ ID NO:21, beginning at the terminal C-terminus amino acid residue, or an amino acid sequence that has at least 75% amino acid identity along its entire length with the sequence of SEQ ID NO:21.

Optionally, the N-terminus amino acid residues of the variant of the DhdR transcription factor are the amino acid residues of SEQ ID NO:22, wherein the amino acid residues are present in the order recited in SEQ ID NO:22, beginning at the terminal N-terminus amino acid residue, or an amino acid sequence that has at least 75% amino acid identity along its entire length with the sequence of SEQ ID NO:22.

Optionally, the C-terminus amino acid residues of the variant of the DhdR transcription factor are the amino acid residues of SEQ ID NO:23, wherein the amino acid residues are present in the order recited in SEQ ID NO:23, beginning at the terminal C-terminus amino acid residue, or an amino acid sequence that has at least 75% amino acid identity along its entire length with the sequence of SEQ ID NO:23.

Most preferably, the variant of DhdR transcription factor comprises an amino acid sequence of SEQ ID NO:2 (Variant 2 truncated DhdR). Optionally, the variant may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:2.Optionally, the variant of DhdR transcription factor comprises an amino acid sequence of SEQ ID NO: 14 (Variant 1 truncated DhdR). Optionally, the variant may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14.

Optionally, the variant of DhdR transcription factor comprises an amino acid sequence of SEQ ID NO: 15 (Variant 3 truncated DhdR). Optionally, the variant may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:15.

### Construct

The construct is preferably a fusion protein.

Most preferably, the fusion protein comprises an amino acid sequence of SEQ ID NO:4. Optionally, the fusion protein may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:4.

Optionally, the fusion protein comprises an amino acid sequence of SEQ ID NO:16. Optionally, the fusion protein may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO: 16.

Optionally, the fusion protein an amino acid sequence of SEQ ID NO:17. Optionally, the fusion protein may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:17.

Optionally, the construct also comprises a nuclear localisation signal.

Optionally, the nuclear localisation signal comprises one or more short amino acid sequences of positively charged lysines or arginines exposed on the protein surface. Optionally, the nuclear localisation signal comprises the amino acid sequence PKKKRKV.

Optionally, the construct comprises the nuclear localization signal (NLS) MPKKKRKVEDA at the N-terminus.

Optionally, the construct also comprises a mitochondrial localisation signal.

Optionally, the mitochondrial localisation signal comprises a mostly alternating pattern of mostly hydrophobic and positively charged amino acid residues that form an amphipathic helix.

Optionally, the construct also comprises four copies of CoxVII (YVRPDAAAAAGLDRLGPAAPSAARQDPFVG) at the N-terminus for the mitochondrial localisation signal.

Optionally, the construct also comprises a nuclear export signal.

Optionally, the nuclear export signal comprises at least 4 hydrophobic amino acid residues.

Optionally, the construct comprises the nuclear export signal (NES) MLQLPPLERLTL at the N-terminus.

### Nucleic Acid Molecules

According to the invention, there is also provided an nucleic acid molecule (such as an isolated nucleic acid molecule) encoding a fusion protein according to the invention, or a nucleic acid molecule (such as an isolated nucleic acid molecule) comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with the nucleic acid molecule over its entire length, or the complement thereof.

Preferably, the nucleic acid molecule (or isolated nucleic acid molecule) comprises a nucleotide sequence of SEQ ID NO:3, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:3 over its entire length, or the complement thereof.

Preferably, the nucleic acid molecule (or isolated nucleic acid molecule) comprises a nucleotide sequence of SEQ ID NO:5, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:5 over its entire length, or the complement thereof.

### Vectors

According to the invention there is also provided a vector comprising a nucleic acid molecule according to the invention.

Optionally, the vector further comprises a promoter operably linked to the nucleic acid. Optionally, the promoter is for expression of a polypeptide encoded by the nucleic acid in a protein expression cell line.

Optionally, the promoter is for expression of a polypeptide encoded by the nucleic acid in mammalian cells.

Optionally, the promoter is for expression of a polypeptide encoded by the nucleic acid in bacterial cells.

Preferably, the vector is a pcDNA3 vector.

Preferably, the vector is a pBAD vector.

Optionally, the promoter is for expression of a polypeptide encoded by the nucleic acid in yeast or insect cells.

Optionally, the vector is a protein expression vector.

Optionally, is a DNA protein expression vector.

According to the invention there is also provided an isolated cell comprising a vector according to the invention.

### Pharmaceutical Compositions

According to the invention there is also provided a pharmaceutical composition comprising a construct (e.g. fusion protein) according to the invention, and a pharmaceutically acceptable carrier, excipient, or diluent.

Preferably, the pharmaceutical composition comprises a polypeptide comprising an amino acid sequence of SEQ ID NO:4. Optionally, the polypeptide may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:4.

Preferably the pharmaceutical composition comprises a polypeptide comprising a an amino acid sequence of SEQ ID NO:16. Optionally, the polypeptide may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO: 16.

Preferably the pharmaceutical composition comprises a polypeptide comprising an amino acid sequence of SEQ ID NO:17. Optionally, the fusion protein may comprise an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:17.

According to the invention there is also provided a pharmaceutical composition comprising a nucleic acid molecule according to the invention, and a pharmaceutically acceptable carrier, excipient, or diluent.

Preferably the pharmaceutical composition comprises a nucleic acid molecule encoding a polypeptide which comprises an amino acid sequence of SEQ ID NO:4, or the complement thereof. Optionally, the nucleic acid molecule encodes a polypeptide with an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:4, or a complement thereof.

Preferably the pharmaceutical composition comprises a nucleic acid molecule encoding a polypeptide which comprises an amino acid sequence of SEQ ID NO:16, or the complement thereof. Optionally, the nucleic acid molecule encodes a polypeptide with an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16, or a complement thereof.

Preferably the pharmaceutical composition comprises a nucleic acid molecule encoding a polypeptide which comprises an amino acid sequence of SEQ ID NO:17, or the complement thereof. Optionally, the nucleic acid molecule encodes a polypeptide with an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO: 17, or a complement thereof.

Preferably the pharmaceutical composition comprises a nucleic acid molecule comprising a nucleotide sequence of SEQ ID NO:5 or a complement thereof, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:5 over its entire length, or a complement thereof.

According to the invention there is also provided a pharmaceutical composition comprising a vector according to the invention, and a pharmaceutically acceptable carrier, excipient, or diluent.

### Sequence Identity

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4:29; program availablefrom http://bitincka.com/ledion/matgat), Gap (Needleman & Wunsch, 1970, J. Mol. Biol. 48: 443-453), FASTA (Altschul et al., 1990, J. Mol. Biol. 215: 403-410; program availablefrom http://www.ebi.ac.uk/fasta), Clustal W 2.0 and X 2.0 (Larkin et al., 2007, Bioinformatics 23: 2947-2948; program availablefrom http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs availablefrom http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

### Methods of Diagnosis

According to the invention, there is provided a method for detecting D-2-HG in a subject, such as in a tissue of a subject, using a construct (e.g. fusion protein) according to the invention.

Detecting D-2-HG may be used to diagnose cancer, such a glioma. For example, it may determine the nature of the tumor, for example the phenotype or genotype of the tumor.

The method may be carried out in vitro, in vivo, or ex vivo. Preferably, the method is carried out in vitro or ex vivo, or on a biological sample obtained from the subject.

According to the invention, there is provided a method for detecting D-2-HG in a biological sample obtained from a subject, using a fusion protein according to the invention

According to the invention there is also provided a method for determining the IDH1/2 mutational status of a biological sample obtained from a subject, wherein the method comprises detecting for D-2-HG in the sample using a fusion protein according to the invention.

According to the invention there is also provided a fusion protein according to the invention, for use in determining the IDH1/2 mutational status of a tissue or cell of a subject in vivo, wherein determining the IDH1/2 mutational status of the sample comprises detecting for D-2-HG in the tissue or cell.

According to the invention there is also provided use of a fusion protein according to the invention, in the manufacture of a medicament for determining the IDH1/2 mutational status of a tissue or cell in a subject in vivo, wherein determining the IDH1/2 mutational status of the sample comprises detecting for D-2-HG in the tissue or cell.

Optionally, detecting for D-2-HG comprises determining the relative difference in fluorescence intensities between the first fluorescent reporter and the second fluorescent reporter, or between the second fluorescent reporter and the first fluorescent reporter.

Optionally, the relative difference in fluorescence is a FRET ratio.

Optionally, the FRET ratio is used to quantify D-2-HG.

Optionally, a FRET ratio of ≥1.7 determines the presence of an IDH1/2 mutation.

Optionally, detecting D-2-HG and/or determining the IDH1/2 mutational status is performed before, with, or after treatment with a medicament.

Optionally, the medicament is an IDH1 inhibitor. Optionally, the IDH1 inhibitor is AG-120.

IDH1 inhibitors may be used to treat cancer, such as glioma.

Optionally, detecting D-2-HG and/or determining the IDH1/2 mutational status is performed before, during, or after surgery.

According to the invention there is provided a method of treating a subject after detecting D-2-HG and/or determining the IDH1/2 mutational status of the subject. The method of treating of treating may involve treatment of cancer, such as glioma. The method of treatment may comprise administering an IDH1 inhibitor.

According to the invention there is also provided a method for monitoring a change in D-2-HG levels in a subject, which comprises:
(i) obtaining a biological sample from a subject;
(ii) detecting for D-2-HG in the sample by determining the relative difference in fluorescence intensities between the first fluorescent reporter and the second fluorescent reporter, or between the second fluorescent reporter and the first fluorescent reporter, in the biological sample using a fusion protein according to the invention;
(iii) repeating steps (i) and (ii) after a period of time; and
(iv) determining whether the relative difference in fluorescence intensities between the first fluorescent reporter and the second fluorescent reporter, or between the second fluorescent reporter and the first fluorescent reporter, has changed.

According to the invention there is also provided a fusion protein according to the invention for use in monitoring a change in D-2-HG levels in a subject in vivo, wherein the monitoring of a change in D-2-HG levels comprises:
(i) detecting D-2-HG by determining the relative difference in fluorescence intensities between the first fluorescent reporter and the second fluorescent reporter, or between the second fluorescent reporter and the first fluorescent reporter, in the biological sample using a fusion protein according to the invention;
(ii) repeating step (i) after a period of time; and
(iii) determining whether the relative difference in fluorescence intensities between the first fluorescent reporter and the second fluorescent reporter, or between the second fluorescent reporter and the first fluorescent reporter, has changed.

According to the invention there is also provided use of a fusion protein according to the invention in a method of manufacture of a medicament for monitoring a change in D-2-HG levels in a subject in vivo, wherein the monitoring of a change in D-2-HG levels comprises:
(i) detecting D-2-HG by determining the relative difference in fluorescence intensities between the first fluorescent reporter and the second fluorescent reporter, or between the second fluorescent reporter and the first fluorescent reporter, in the biological sample using a fusion protein according to any of claims 1 to 153;
(ii) repeating step (i) after a period of time; and
(iii) determining whether the relative difference in fluorescence intensities between the first fluorescent reporter and the second fluorescent reporter, or between the second fluorescent reporter and the first fluorescent reporter, has changed.

Optionally, monitoring a change in D-2-HG levels in a subject is performed before, with, or after treatment with a medicament.

Optionally, monitoring a change in D-2-HG levels is performed before, during, or after surgery.

Optionally, the biological sample is a cell culture, plasma, serum, tumor, urine, cerebrospinal fluid, or artificial cerebrospinal fluid sample.

Optionally, the tumor sample is a glioma tumor sample.

Optionally, the tumor sample is a tumor lysates sample.

Optionally, the cell culture sample is a cell culture supernatant sample.

Optionally, the D-2-HG is detected for in the cytoplasm, mitochondria, or nucleus of the cell.

Optionally, the fluorescence intensities from the first and/or second fluorescent reporter are detected using a fluorescence microscope.

According to the invention there is also provided a method of analysing D-2-HG in a biological sample obtained from a subject, comprising detecting for D-2-HG in the cytoplasm or subcellular compartments of a cell in the biological sample using a construct which comprises a DhdR transcription factor or variant thereof linked to a first fluorescent reporter and a second fluorescent reporter.

According to the invention there is also provided a construct for use in analysing D-2-HG in a cell of a subject in vivo, wherein the use comprises detecting for D-2-HG in the cytoplasm or subcellular compartments of the cell, wherein the construct comprises a DhdR transcription factor or variant thereof linked to a first fluorescent reporter and a second fluorescent reporter.

According to the invention there is also provided use of a construct in the manufacture of a medicament for analysing D-2-HG in a cell of a subject in vivo, wherein the construct comprises a DhdR transcription factor or variant thereof linked to a first fluorescent reporter and a second fluorescent reporter, and wherein the use comprises detecting for D-2-HG in the cytoplasm or subcellular compartments of the cell.

Optionally, detecting for D-2-HG in the cytoplasm or subcellular compartments of the cell comprises determining the relative difference in fluorescence intensities between the first fluorescent reporter and the second fluorescent reporter, or between the second fluorescent reporter and the first fluorescent reporter.

Optionally, wherein detecting for the D-2-HG in the cytoplasm or subcellular compartments of the cell in the biological sample determines the subcellular distribution of D-2-HG.

Optionally, the subcellular location is the mitochondria, cytoplasm, or the nucleus.

Optionally, the construct also comprises a nuclear localisation signal.

Optionally, the nuclear localisation signal comprises one or more short amino acid sequences of positively charged lysines or arginines exposed on the protein surface. Optionally, the nuclear localisation signal comprises the amino acid sequence PKKKRKV.

Optionally, the construct comprises the nuclear localization signal (NLS) MPKKKRKVEDA at the N-terminus.

Optionally, the construct also comprises a mitochondrial localisation signal.

Optionally, the mitochondrial localisation signal comprises a mostly alternating pattern of mostly hydrophobic and positively charged amino acid residues that form an amphipathic helix.

Optionally, the construct also comprises four copies of CoxVII (YVRPDAAAAAGLDRLGPAAPSAARQDPFVG) at the N-terminus for the mitochondrial localisation signal.

Optionally, the construct also comprises a nuclear export signal.

Optionally, the nuclear export signal comprises at least 4 hydrophobic amino acid residues.

Optionally, the construct comprises the nuclear export signal (NES) MLQLPPLERLTL at the N-terminus.

Aspects of the invention are defined in the following numbered paragraphs:
1. A construct for detection of d-2-hydroxyglutarate (d-2-HG), comprising:
   a first reporter;
   a second reporter; and
   a DhdR transcription factor, or variant thereof,
   wherein the first reporter is linked at the N-terminus of the DhdR transcription factor, or variant thereof, and the second reporter is linked at the C-terminus of the DhdR transcription factor, or variant thereof, or wherein the first reporter is linked at the C-terminus of the DhdR transcription factor, or variant thereof, and the second reporter is linked at the N-terminus of the DhdR transcription factor, or variant thereof, and
   wherein the variant of the DhdR transcription factor comprises a truncated DhdR transcription factor for binding d-2-HG, in which up to 5 amino acid residues are truncated from the N-terminus of DhdR corresponding to the terminal N-terminus amino acid residues of DhdR, and/or up to 5 amino acid residues are truncated from the C-terminus of DhdR corresponding to the terminal C-terminus amino acid residues of DhdR,
   optionally wherein the first reporter is a first fluorescent reporter, preferably having an emission peak which is less than 490 nm, and the second reporter is a second fluorescent reporter preferably having an excitation peak which is less than 550 nm.
2. A construct according to paragraph 1, wherein the first reporter is linked at the N-terminus of the DhdR transcription factor, or variant thereof, and the second reporter is linked at the C-terminus of the DhdR transcription factor, or variant thereof.
3. A construct according to paragraph 1, wherein the first reporter is linked at the C-terminus of the DhdR transcription factor, or variant thereof, and the second reporter is linked at the N-terminus of the DhdR transcription factor, or variant thereof.
4. A construct according to any of paragraphs 1 to 3, wherein the first reporter is linked at the N-terminus of the DhdR transcription factor, or variant thereof, by a first linker which comprises at least one amino acid residue, and the second reporter is linked at the C-terminus of the DhdR transcription factor, or variant thereof, by a second linker which comprises at least one amino acid residue.
5. A construct according to any of paragraphs 1 to 3, wherein the first reporter is linked at the C-terminus of the DhdR transcription factor, or variant thereof, by a first linker which comprises at least one amino acid residue, and the second reporter is linked at the N-terminus of the DhdR transcription factor, or variant thereof, by a second linker which comprises at least one amino acid residue.
6. A construct according to paragraph 4 or 5, wherein the first linker comprises two amino acid residues.
7. A construct according to any of paragraphs 4 to 6, wherein the second linker comprises two amino acid residues.
8. A construct according to any of paragraphs 4 to 7 wherein the first linker comprises three amino acid residues.
9. A construct according to any of paragraphs 4 to 8, wherein the second linker comprises three amino acid residues.
10. A construct according to any of paragraphs 4 to 9, wherein the first linker comprises four amino acid residues.
11. A construct according to any of paragraphs 4 to 10, wherein the second linker comprises four amino acid residues.
12. A construct according to any of paragraphs 4 to 11, wherein the first linker comprises five amino acid residues.
13. A construct according to any of paragraphs 4 to 12, wherein the second linker comprises five amino acid residues.
14. A construct according to any of paragraphs 4 to 13, wherein the first linker comprises an amino acid R.
15. A construct according to any of paragraphs 4 to 14, wherein the first linker comprises an amino acid sequence of R and M.
16. A construct according to any of paragraphs 4 to 15, wherein the first linker comprises an amino acid sequence of R, M, and H.
17. A construct according to any of paragraphs 4 to 16, wherein the second linker comprises an amino acid R.
18. A construct according to any of paragraphs 4 to 17, wherein the second linker comprises an amino acid sequence of R and M.
19. A construct according to any of paragraphs 4 to 18, wherein the second linker comprises an amino acid sequence of R, M, and H.
20. A construct according to any of paragraphs 4 to 16, wherein the second linker comprises an amino acid E.
21. A construct according to any of paragraphs 4 to 16, or 20, wherein the second linker comprises an amino acid sequence of E, and L.
22. A construct according to any of paragraphs 4 to 13, or 17 to 21, wherein the first linker comprises an amino acid E.
23. A construct according to any of paragraphs 4 to 13, or 17 to 22, wherein the first linker comprises an amino acid sequence of E, and L.
24. A construct according to any preceding paragraph, wherein the first and the second reporter comprise first and second fluorescent proteins, respectively.
25. A construct according to paragraph 24, wherein the first fluorescent protein is enhanced cyan fluorescent protein (ECFP).
26. A construct according to paragraph 24 or 25, wherein the second fluorescent protein is cpVenus173.
27. A construct according to paragraph 24, wherein the first fluorescent protein is cpVenus173.
28. A construct according to paragraph 24 or 27, wherein the second fluorescent protein is ECFP.
29. A construct according to any preceding paragraph, in which one amino acid residue is truncated from the N-terminus of the truncated DhdR transcription factor, corresponding to the terminal N-terminus amino acid residue of the DhdR transcription factor.
30. A construct according to any preceding paragraph, in which one amino acid residue is truncated from the C-terminus of the truncated DhdR transcription factor, corresponding to the terminal C-terminus amino acid residue of the DhdR transcription factor.
31. A construct according to any of paragraphs 1 to 28, in which two amino acid residues are truncated from the N-terminus of the truncated DhdR transcription factor, corresponding to the two terminal N-terminus amino acid residues of DhdR transcription factor.
32. A construct according to any of paragraphs 1 to 28, in which two amino acid residues are truncated from the C-terminus of the truncated DhdR transcription factor, corresponding to the two terminal C-terminus amino acid residues of DhdR transcription factor.
33. A construct according to any of paragraphs 1 to 28, in which three amino acid residues are truncated from the N-terminus of the truncated DhdR transcription factor, corresponding to the three terminal N-terminus amino acid residues of DhdR transcription factor.
34. A construct according to any of paragraphs 1 to 28, in which three amino acid residues are truncated from the C-terminus of the truncated DhdR transcription factor, corresponding to the three terminal C-terminus amino acid residues of DhdR transcription factor.
35. A construct according to any of paragraphs 1 to 28, in which four amino acid residues are truncated from the N-terminus of the truncated DhdR transcription factor, corresponding to the four terminal N-terminus amino acid residues of DhdR transcription factor.
36. A construct according to any of paragraphs 1 to 28, in which four amino acid residues are truncated from the C-terminus of the truncated DhdR transcription factor, corresponding to the four terminal C-terminus amino acid residues of DhdR transcription factor.
37. A construct according to any of paragraphs 1 to 28, in which five amino acid residues are truncated from the N-terminus of the truncated DhdR transcription factor, corresponding to the five terminal N-terminus amino acid residues of DhdR transcription factor.
38. A construct according to any of paragraphs 1 to 28, in which five amino acid residues are truncated from the C-terminus of the truncated DhdR transcription factor, corresponding to the five terminal C-terminus amino acid residues of DhdR transcription factor.

39. A construct according to any of paragraphs 1 to 30, in which one amino acid residue is truncated from the N-terminus of the truncated DhdR transcription factor, corresponding to the terminal N-terminus amino acid residue of DhdR transcription factor, and one amino acid residue is truncated from the C-terminus of the truncated DhdR transcription factor, corresponding to the terminal C-terminus amino acid residue of DhdR transcription factor.
40. A construct according to any of paragraphs 1 to 28, 31, or 32, in which two amino acid residues are truncated from the N-terminus of the truncated DhdR transcription factor, corresponding to the two terminal N-terminus amino acid residues of DhdR, and two amino acid residues are truncated from the C-terminus of the truncated DhdR transcription factor, corresponding to the two terminal C-terminus amino acid residues of DhdR transcription factor.
41. A construct according to any of paragraphs 1 to 28, 33, or 34, in which three amino acid residues are truncated from the N-terminus of the truncated DhdR transcription factor, corresponding to the three terminal N-terminus amino acid residues of DhdR transcription factor, and three amino acid residues are truncated from the C-terminus of the truncated DhdR transcription factor, corresponding to the three terminal C-terminus amino acid residues of DhdR transcription factor.
42. A construct according to any of paragraphs 1 to 28, 31, 32, or 40, in which the N-terminus amino acid residues of the truncated DhdR transcription factor are the amino acid residues of SEQ ID NO:18, wherein the amino acid residues are present in the order recited in SEQ ID NO:18, beginning at the terminal N-terminus amino acid residue, or an amino acid sequence that has at least 75% amino acid identity along its entire length with the sequence of SEQ ID NO:18.
43. A construct according to any of paragraphs 1 to 28, 31, 32, 40, or 42, in which the C-terminus amino acid residues of the truncated DhdR transcription factor are the amino acid residues of SEQ ID NO:19, wherein the amino acid residues are present in the order recited in SEQ ID NO:19, beginning at the terminal C-terminus amino acid residue, or an amino acid sequence that has at least 75% amino acid identity along its entire length with the sequence of SEQ ID NO:19.
44. A construct according to any of paragraphs 1 to 28, 33, 34, or 41, in which the N-terminus amino acid residues of the truncated DhdR transcription factor are the amino acid residues of SEQ ID NO:20, wherein the amino acid residues are present in the order recited in SEQ ID NO:20, beginning at the terminal N-terminus amino acid residue, or an amino acid sequence that has at least 75% amino acid identity along its entire length with the sequence of SEQ ID NO:20.
45. A construct according to any of paragraphs 1 to 28, 33, 34, 41, or 44, in which the C-terminus amino acid residues of the truncated DhdR transcription factor are the amino acid residues of SEQ ID NO:21, wherein the amino acid residues are present in the order recited in SEQ ID NO:21, beginning at the terminal C-terminus amino acid residue, or an amino acid sequence that has at least 75% amino acid identity along its entire length with the sequence of SEQ ID NO:21.
46. A construct according to any of paragraphs 1 to 30, or 39, in which the N-terminus amino acid residues of the truncated DhdR transcription factor are the amino acid residues of SEQ ID NO:22, wherein the amino acid residues are present in the order recited in SEQ ID NO: 22, beginning at the terminal N-terminus amino acid residue, or an amino acid sequence that has at least 75% amino acid identity along its entire length with the sequence of SEQ ID NO:22
47. A construct according to any of paragraphs 1 to 30, 39, or 46, in which the C-terminus amino acid residues of the truncated DhdR transcription factor are the amino acid residues of SEQ ID NO:23, wherein the amino acid residues are present in the order recited in SEQ ID NO: 23, beginning at the terminal C-terminus amino acid residue, or an amino acid sequence that has at least 75% amino acid identity along its entire length with the sequence of SEQ ID NO:23.
48. A construct according to any of paragraphs 1 to 28, 31, 32, 40, 42, 43, wherein the truncated DhdR transcription factor comprises an amino acid sequence of SEQ ID NO:2 (Variant 2 truncated DhdR).
49. A construct according to any of paragraphs 1 to 30, 39, 46, or 47, wherein the truncated DhdR transcription factor comprises an amino acid sequence of SEQ ID NO:14 (Variant 1 truncated DhdR).
50. A construct according to any of paragraphs 1 to 28, 33, 34, 41, 44, or 45, wherein the truncated DhdR transcription factor comprises an amino acid sequence of SEQ ID NO:15 (Variant 3 truncated DhdR).
51. A construct according to any of paragraphs 1 to 28, wherein the DhdR transcription factor comprises an amino acid sequence of SEQ ID NO:1.
52. A construct according to any of paragraphs 1 to 28, 31, 32, 40, 42, 43, or 48, which comprises an amino acid sequence of SEQ ID NO:4.
53. A construct according to any of paragraphs 1 to 30, 39, 46, 47, or 49 which comprises an amino acid sequence of SEQ ID NO: 16.
54. A construct according to any of paragraphs 1 to 28, 33, 34, 41, 44, 45, or 50, which comprises an amino acid sequence of SEQ ID NO: 17.
55. A construct according to paragraph 24 or 28, wherein the amino acid sequence of ECFP comprises amino acid sequence of SEQ ID NO:6.
56. A construct according to paragraph 26 or 27, wherein the amino acid sequence of cpVenus173 comprises amino acid sequence of SEQ ID NO:8.
57. A construct for detection of d-2-hydroxyglutarate (d-2-HG), comprising
   a DhdR transcription factor, or variant thereof,
   a first reporter, preferably a first fluorescent reporter;
   a second reporter, preferably a second fluorescent reporter
   a first linker;
   a second linker;
   wherein the first reporter is linked at the N-terminus of the DhdR transcription factor, or variant thereof, and the second reporter is linked at the C-terminus of the DhdR transcription factor, or variant thereof, or wherein the first reporter is linked at the C-terminus of the DhdR transcription factor, or variant thereof, and the second reporter is linked at the N-terminus of the DhdR transcription factor, or variant thereof,
   wherein the variant of the DhdR transcription factor comprises a truncated DhdR transcription factor for binding d-2-HG, in which up to 5 amino acid residues are truncated from the N-terminus of DhdR corresponding to the terminal N-terminus amino acid residues of DhdR, and/or up to 5 amino acid residues are truncated from the C-terminus of DhdR corresponding to the terminal C-terminus amino acid residues of DhdR, and
   wherein the first linker comprises an amino acid sequence of R, M, and H, and the second linker comprises an amino acid sequence of E, and L, or wherein the first linker comprises an amino acid sequence of E, and L, and the second linker comprises an amino acid sequence of R, M, and H.
58. A construct according to paragraph 57, wherein the first reporter is linked at the N-terminus of the DhdR transcription factor, or variant thereof, and the second reporter is linked at the C-terminus of the a DhdR transcription factor, or variant thereof.
59. A construct according to paragraph 57, wherein the first reporter is linked at the C-terminus of the a DhdR transcription factor, or variant thereof, and the second reporter is linked at the N-terminus of the a DhdR transcription factor, or variant thereof.
60. A construct according to any of paragraphs 57 to 59, wherein the first linker comprises an amino acid sequence of R, M, and H, and the second linker comprises an amino acid sequence of E, and L.
61. A construct according to any of paragraphs 57 to 59, wherein the first linker comprises an amino acid sequence E, and L, and the second linker comprises an amino acid sequence of R, M, and H.
62. A construct according to any of paragraphs 57 to 61, wherein the first and the second reporter comprise a first and a second fluorescent protein, respectively.
63. A construct according to paragraph 62, wherein the first fluorescent protein has an emission peak which is less than 500 nm.
64. A construct according to paragraph 62 or 63, wherein the second fluorescent protein has an excitation peak which is less than 490 nm.
65. A construct according to paragraph 62, wherein the first fluorescent protein has an excitation peak which is less than 550 nm.
66. A construct according to paragraph 62 or 65, wherein the second fluorescent protein has an emission peak which is less than 500 nm.
67. A construct according to paragraph 62 or 63, wherein the first fluorescent protein is enhanced cyan fluorescent protein (ECFP).
68. A construct according to paragraph 62, 63, 64 or 67, wherein the second fluorescent protein is cpVenus173.
69. A construct according to paragraph 62 or 65, wherein the first fluorescent protein is cpVenus173.
70. A construct according to paragraph 62, 65, 66, or 69, wherein the second fluorescent protein is ECFP.
71. A construct according to any of paragraphs 67 or 70, wherein the amino acid sequence of ECFP comprises amino acid sequence of SEQ ID NO:6.
72. A construct according to any of paragraphs 68 or 69, wherein the amino acid sequence of cpVenus173 comprises amino acid sequence of SEQ ID NO:8.
73. A construct according to any of paragraphs 57 to 72, in which one amino acid residue is truncated from the N-terminus of the truncated DhdR transcription factor, corresponding to the terminal N-terminus amino acid residue of the DhdR transcription factor.
74. A construct according to any of paragraphs 57 to 73, in which one amino acid residue is truncated from the C-terminus of the truncated DhdR transcription factor, corresponding to the terminal C-terminus amino acid residue of the DhdR transcription factor.
75. A construct according to any of paragraphs 57 to 72, in which two amino acid residues are truncated from the N-terminus of the truncated DhdR transcription factor, corresponding to the two terminal N-terminus amino acid residues of DhdR transcription factor.
76. A construct according to any of paragraphs 57 to 72, in which two amino acid residues are truncated from the C-terminus of the truncated DhdR transcription factor, corresponding to the two terminal C-terminus amino acid residues of DhdR transcription factor.
77. A construct according to any of paragraphs 57 to 72, in which three amino acid residues are truncated from the N-terminus of the truncated DhdR transcription factor, corresponding to the three terminal N-terminus amino acid residues of DhdR transcription factor.
78. A construct according to any of paragraphs 57 to 72, in which three amino acid residues are truncated from the C-terminus of the truncated DhdR transcription factor, corresponding to the three terminal C-terminus amino acid residues of DhdR transcription factor.
79. A construct according to any of paragraphs 57 to 72, in which four amino acid residues are truncated from the N-terminus of the truncated DhdR transcription factor, corresponding to the four terminal N-terminus amino acid residues of DhdR transcription factor.
80. A construct according to any of paragraphs 57 to 72, in which four amino acid residues are truncated from the C-terminus of the truncated DhdR transcription factor, corresponding to the four terminal C-terminus amino acid residues of DhdR transcription factor.
81. A construct according to any of paragraphs 57 to 72, in which five amino acid residues are truncated from the N-terminus of the truncated DhdR transcription factor, corresponding to the five terminal N-terminus amino acid residues of DhdR transcription factor.
82. A construct according to any of paragraphs 57 to 72, in which five amino acid residues are truncated from the C-terminus of the truncated DhdR transcription factor, corresponding to the five terminal C-terminus amino acid residues of DhdR transcription factor.
83. A construct according to any of paragraphs 57 to 74, in which one amino acid residue is truncated from the N-terminus of the truncated DhdR transcription factor, corresponding to the terminal N-terminus amino acid residue of DhdR transcription factor, and one amino acid residue is truncated from the C-terminus of the truncated DhdR transcription factor, corresponding to the terminal C-terminus amino acid residue of DhdR transcription factor.
84. A construct according to any of paragraphs 57 to 72, 75, or 76, in which two amino acid residues are truncated from the N-terminus of the truncated DhdR transcription factor, corresponding to the two terminal N-terminus amino acid residues of DhdR, and two amino acid residues are truncated from the C-terminus of the truncated DhdR transcription factor, corresponding to the two terminal C-terminus amino acid residues of DhdR transcription factor.
85. A construct according to any of paragraphs 57 to 72, 77, or 78, in which three amino acid residues are truncated from the N-terminus of the truncated DhdR transcription factor, corresponding to the three terminal N-terminus amino acid residues of DhdR transcription factor, and three amino acid residues are truncated from the C-terminus of the truncated DhdR transcription factor, corresponding to the three terminal C-terminus amino acid residues of DhdR transcription factor.
86. A construct according to any of paragraphs 57 to 72, 75, 76, or 84, in which the N-terminus amino acid residues of the truncated DhdR transcription factor are the amino acid residues of SEQ ID NO:18, wherein the amino acid residues are present in the order recited in SEQ ID NO:18, beginning at the terminal N-terminus amino acid residue, or an amino acid sequence that has at least 75% amino acid identity along its entire length with the sequence of SEQ ID NO:18.
87. A construct according to any of paragraphs 57 to 72, 75, 76, 84, or 86, in which the C-terminus amino acid residues of the truncated DhdR transcription factor are the amino acid residues of SEQ ID NO:19, wherein the amino acid residues are present in the order recited in SEQ ID NO:19, beginning at the terminal C-terminus amino acid residue, or an amino acid sequence that has at least 75% amino acid identity along its entire length with the sequence of SEQ ID NO:19.
88. A construct according to any of paragraphs 57 to 72, 77, 78, or 85, in which the N-terminus amino acid residues of the truncated DhdR transcription factor are the amino acid residues of SEQ ID NO:20, wherein the amino acid residues are present in the order recited in SEQ ID NO:20, beginning at the terminal N-terminus amino acid residue, or an amino acid sequence that has at least 75% amino acid identity along its entire length with the sequence of SEQ ID NO:20.
89. A construct according to any of paragraphs 57 to 72, 77, 78, 85, or 88, in which the C-terminus amino acid residues of the truncated DhdR transcription factor are the amino acid residues of SEQ ID NO:21, wherein the amino acid residues are present in the order recited in SEQ ID NO:21, beginning at the terminal C-terminus amino acid residue, or an amino acid sequence that has at least 75% amino acid identity along its entire length with the sequence of SEQ ID NO:21.
90. A construct according to any of paragraphs 57 to 74, or 83, in which the N-terminus amino acid residues of the truncated DhdR transcription factor are the amino acid residues of SEQ ID NO:22, wherein the amino acid residues are present in the order recited in SEQ ID NO: 22, beginning at the terminal N-terminus amino acid residue, or an amino acid sequence that has at least 75% amino acid identity along its entire length with the sequence of SEQ ID NO:22
91. A construct according to any of paragraphs 57 to 74, 83, or 90, in which the C-terminus amino acid residues of the truncated DhdR transcription factor are the amino acid residues of SEQ ID NO:23, wherein the amino acid residues are present in the order recited in SEQ ID NO:23, beginning at the terminal C-terminus amino acid residue, or an amino acid sequence that has at least 75% amino acid identity along its entire length with the sequence of SEQ ID NO:23.
92. A construct according to any of paragraphs 57 to 72, 75, 76, 84, 86, or 87, wherein the truncated DhdR transcription factor comprises an amino acid sequence of SEQ ID NO:2 (Variant 2 truncated DhdR).
93. A construct according to any of paragraphs 57 to 74, 83, 90, 91, wherein the truncated DhdR transcription factor comprises an amino acid sequence of SEQ ID NO:14 (Variant 1 truncated DhdR).
94. A construct according to any of paragraphs 57 to 72, 77, 78, 85, 88, or 89, wherein the truncated DhdR transcription factor comprises an amino acid sequence of SEQ ID NO:15 (Variant 3 truncated DhdR).
95. A construct according to any of paragraphs 1 to 72, wherein the DhdR transcription factor comprises an amino acid sequence of SEQ ID NO:1.
96. A construct according to any of paragraphs 57 to 72, 75, 76, 84, 86, 87, or 92, which comprises an amino acid sequence of SEQ ID NO:4.
97. A construct according to any of paragraphs 57 to 74, 83, 90, 91 or 93, which comprises an amino acid sequence of SEQ ID NO:16.
98. A construct according to any of paragraphs 57 to 72, 77, 78, 85, 88, 89, or 94, which comprises an amino acid sequence of SEQ ID NO:17.
99. A construct for detection of d-2-hydroxyglutarate (d-2-HG), comprising:
   an amino acid sequence of SEQ ID NO:1 (full length DhdR), or variant thereof;
   a first reporter, preferably a first fluorescent reporter; and
   a second reporter, preferably a second fluorescent reporter;
   wherein the first reporter is linked at the N-terminus of the amino acid sequence of SEQ ID NO:1, and the second reporter is linked at the C-terminus of the amino acid sequence of SEQ ID NO:1, or wherein the first reporter is linked at the C-terminus of the amino acid sequence of SEQ ID NO:1, and the second reporter is linked at the N-terminus of the amino acid sequence of SEQ ID NO:1,
   wherein the variant of the amino acid sequence of SEQ ID NO:1 comprises a truncation, in which up to 5 amino acid residues are truncated from the N-terminus of the amino acid sequence of SEQ ID NO:1 corresponding to the terminal N-terminus amino acid residues of the amino acid sequence of SEQ ID NO:1, and/or up to 5 amino acid residues are truncated from the C-terminus of the amino acid sequence of SEQ ID NO:1 corresponding to the terminal C-terminus amino acid residues of the amino acid sequence of SEQ ID NO:1.
100. A construct according to paragraph 99, wherein the first reporter is linked at the N-terminus of the amino acid sequence of SEQ ID NO:1, or variant thereof, and the second reporter is linked at the C-terminus of the DhdR transcription factor, or variant thereof.
101. A construct according to paragraph 99, wherein the first reporter is linked at the C-terminus of the amino acid sequence of SEQ ID NO:1, or variant thereof, and the second reporter is linked at the N-terminus of the DhdR transcription factor, or variant thereof.
102. A construct according to an of paragraphs 99-101, wherein the first reporter is linked at the N-terminus of the amino acid sequence of SEQ ID NO:1, or variant thereof, by a first linker which comprises at least one amino acid residue, and the second reporter is linked at the C-terminus of the amino acid sequence of SEQ ID NO:1, or variant thereof, by a second linker which comprises at least one amino acid residue.
103. A construct according to any of paragraphs 99-102, wherein the first reporter is linked at the C-terminus of the amino acid sequence of SEQ ID NO:1, or variant thereof, by a first linker which comprises at least one amino acid residue, and the second reporter is linked at the N-terminus of the amino acid sequence of SEQ ID NO:1, or variant thereof, by a second linker which comprises at least one amino acid residue.
104. A construct according to paragraph 102 or 103, wherein the first linker comprises two amino acid residues.
105. A construct according to any of paragraphs 102 to 104, wherein the second linker comprises two amino acid residues.
106. A construct according to any of paragraphs 102 to 105 wherein the first linker comprises three amino acid residues.
107. A construct according to any of paragraphs 102 to 106, wherein the second linker comprises three amino acid residues.
108. A construct according to any of paragraphs 102 to 107, wherein the first linker comprises four amino acid residues.
109. A construct according to any of paragraphs 102 to 108, wherein the second linker comprises four amino acid residues.
110. A construct according to any of paragraphs 102 to 109, wherein the first linker comprises five amino acid residues.
111. A construct according to any of paragraphs 102 to 110, wherein the second linker comprises five amino acid residues.
112. A construct according to any of paragraphs 102 to 111, wherein the first linker comprises an amino acid R.
113. A construct according to any of paragraphs 102 to 112, wherein the first linker comprises an amino acid sequence of R and M.
114. A construct according to any of paragraphs 102 to 113, wherein the first linker comprises an amino acid sequence of R, M, and H.
115. A construct according to any of paragraphs 102 to 114, wherein the second linker comprises an amino acid R.
116. A construct according to any of paragraphs 102 to 115, wherein the second linker comprises an amino acid sequence of R and M.
117. A construct according to any of paragraphs 102 to 116, wherein the second linker comprises an amino acid sequence of R, M, and H.
118. A construct according to any of paragraphs 102 to 114, wherein the second linker comprises an amino acid E.
119. A construct according to any of paragraphs 102 to 114, or 118, wherein the second protein comprises an amino acid sequence of E, and L.
120. A construct according to any of paragraphs 102 to 111, or 112 to 119, wherein the first linker comprises an amino acid E.
121. A construct according to any of paragraphs 102 to 111, or 112 to 120, wherein the first linker comprises an amino acid sequence of E, and L.
122. A construct according to any of paragraphs 99 to 121, wherein the first and the second reporter comprise first and second fluorescent proteins, respectively.
123. A construct according to paragraph 122, wherein the first fluorescent protein is enhanced cyan fluorescent protein (ECFP).
124. A construct according to paragraph 122 or 123, wherein the second fluorescent protein is cpVenus173.
125. A construct according to paragraph 122, wherein the first fluorescent protein is cpVenus173.
126. A construct according to paragraph 122 or 123, wherein the second fluorescent protein is ECFP.
127. A construct according to paragraph 123 or 126, wherein the amino acid sequence of ECFP comprises amino acid sequence of SEQ ID NO:6.
128. A construct according to paragraph 124 or 125, wherein the amino acid sequence of cpVenus173 comprises amino acid sequence of SEQ ID NO:8.
129. A construct according to any of paragraphs 99 to 128, in which one amino acid residue is truncated from the variant of the amino acid sequence of SEQ ID NO:1, corresponding to the terminal N-terminus amino acid residue of the amino acid sequence of SEQ ID NO:1.
130. A construct according to any of paragraphs 99 to 129, in which one amino acid residue is truncated from the C-terminus of the variant of the amino acid sequence of SEQ ID NO: 1, corresponding to the terminal C-terminus amino acid residue of the amino acid sequence of SEQ ID NO:1.
131. A construct according to any of paragraphs 99 to 128, in which two amino acid residues are truncated from the N-terminus of the variant of the amino acid sequence of SEQ ID NO: 1, corresponding to the two terminal N-terminus amino acid residues of the amino acid sequence of SEQ ID NO:1.
132. A construct according to any of paragraphs 99 to 128, in which two amino acid residues are truncated from the C-terminus of the variant of the amino acid sequence of SEQ ID NO: 1, corresponding to the two terminal C-terminus amino acid residues of the amino acid sequence of SEQ ID NO:1.
133. A construct according to any of paragraphs 99 to 128, in which three amino acid residues are truncated from the N-terminus of the variant of the amino acid sequence of SEQ ID NO: 1, corresponding to the three terminal N-terminus amino acid residues of the amino acid sequence of SEQ ID NO:1.
134. A construct according to any of paragraphs 99 to 128, in which three amino acid residues are truncated from the C-terminus of the variant of the amino acid sequence of SEQ ID NO: 1, corresponding to the three terminal C-terminus amino acid residues of the amino acid sequence of SEQ ID NO:1.
135. A construct according to any of paragraphs 99 to 128, in which four amino acid residues are truncated from the N-terminus of the variant of the amino acid sequence of SEQ ID NO: 1, corresponding to the four terminal N-terminus amino acid residues of the amino acid sequence of SEQ ID NO:1.
136. A construct according to any of paragraphs 99 to 128, in which four amino acid residues are truncated from the C-terminus of the variant of the amino acid sequence of SEQ ID NO: 1, corresponding to the four terminal C-terminus amino acid residues of the amino acid sequence of SEQ ID NO:1.
137. A construct according to any of paragraphs 99 to 128, in which five amino acid residues are truncated from the N-terminus of the variant of the amino acid sequence of SEQ ID NO: 1, corresponding to the five terminal N-terminus amino acid residues of DhdR transcription factor.
138. A construct according to any of paragraphs 99 to 128, in which five amino acid residues are truncated from the C-terminus of the variant of the amino acid sequence of SEQ ID NO: 1, corresponding to the five terminal C-terminus amino acid residues of the amino acid sequence of SEQ ID NO:1.
139. A construct according to any of paragraphs 99 to 130, in which one amino acid residue is truncated from the N-terminus of the variant of the amino acid sequence of SEQ ID NO: 1, corresponding to the terminal N-terminus amino acid residue of DhdR transcription factor, and one amino acid residue is truncated from the C-terminus of the truncated DhdR transcription factor, corresponding to the terminal C-terminus amino acid residue of the amino acid sequence of SEQ ID NO:1.
140. A construct according to any of paragraphs 99 to 128, 131, or 132, in which two amino acid residues are truncated from the N-terminus of the variant of the amino acid sequence of SEQ ID NO:1, corresponding to the two terminal N-terminus amino acid residues of DhdR, and two amino acid residues are truncated from the C-terminus of the truncated DhdR transcription factor, corresponding to the two terminal C-terminus amino acid residues of the amino acid sequence of SEQ ID NO:1.
141. A construct according to any of paragraphs 99 to 128, 133, or 134, in which three amino acid residues are truncated from the N-terminus of the variant of the amino acid sequence of SEQ ID NO:1, corresponding to the three terminal N-terminus amino acid residues of DhdR transcription factor, and three amino acid residues are truncated from the C-terminus of the truncated DhdR transcription factor, corresponding to the three terminal C-terminus amino acid residues of the amino acid sequence of SEQ ID NO:1.
142. A construct according to any of paragraphs 99 to 128, 131, 132, or 140, in which the N-terminus amino acid residues of the truncated DhdR transcription factor are the amino acid residues of SEQ ID NO:18, wherein the amino acid residues are present in the order recited in SEQ ID NO:18, beginning at the terminal N-terminus amino acid residue, or an amino acid sequence that has at least 75% amino acid identity along its entire length with the sequence of SEQ ID NO:18.
143. A construct according to any of paragraphs 99 to 128, 131, 132, 140, or 142, in which the C-terminus amino acid residues of the truncated DhdR transcription factor are the amino acid residues of SEQ ID NO:19, wherein the amino acid residues are present in the order recited in SEQ ID NO:19, beginning at the terminal C-terminus amino acid residue, or an amino acid sequence that has at least 75% amino acid identity along its entire length with the sequence of SEQ ID NO:19.
144. A construct according to any of paragraphs 99 to 128, 133, 134, or 141, in which the N-terminus amino acid residues of the truncated DhdR transcription factor are the amino acid residues of SEQ ID NO:20, wherein the amino acid residues are present in the order recited in SEQ ID NO:20, beginning at the terminal N-terminus amino acid residue, or an amino acid sequence that has at least 75% amino acid identity along its entire length with the sequence of SEQ ID NO:20.
145. A construct according to any of paragraphs 99 to 128, 133, 134, 141, or 144, in which the C-terminus amino acid residues of the truncated DhdR transcription factor are the amino acid residues of SEQ ID NO:21, wherein the amino acid residues are present in the order recited in SEQ ID NO:21, beginning at the terminal C-terminus amino acid residue, or an amino acid sequence that has at least 75% amino acid identity along its entire length with the sequence of SEQ ID NO:21.
146. A construct according to any of paragraphs 99 to 130, or 139, in which the N-terminus amino acid residues of the truncated DhdR transcription factor are the amino acid residues of SEQ ID NO:22, wherein the amino acid residues are present in the order recited in SEQ ID NO: 22, beginning at the terminal N-terminus amino acid residue, or an amino acid sequence that has at least 75% amino acid identity along its entire length with the sequence of SEQ ID NO:22
147. A construct according to any of paragraphs 99 to 130, 139, or 146, in which the C-terminus amino acid residues of the truncated DhdR transcription factor are the amino acid residues of SEQ ID NO:23, wherein the amino acid residues are present in the order recited in SEQ ID NO:23, beginning at the terminal C-terminus amino acid residue, or an amino acid sequence that has at least 75% amino acid identity along its entire length with the sequence of SEQ ID NO:23.
148. A construct according to any of paragraphs 99 to 128, 131, 132, 140, 142, 143, wherein the truncated DhdR transcription factor comprises an amino acid sequence of SEQ ID NO: 2 (Variant 2 truncated DhdR).
149. A construct according to any of paragraphs 99 to 130, 139, 146, or 147, wherein the truncated DhdR transcription factor comprises an amino acid sequence of SEQ ID NO:14 (Variant 1 truncated DhdR).
150. A construct according to any of paragraphs 99 to 128, 133, 134, 141, 144, or 145, wherein the truncated DhdR transcription factor comprises an amino acid sequence of SEQ ID NO: 15 (Variant 3 truncated DhdR).
151. A construct according to any of paragraphs 99 to 128, 131, 132, 140, 142, 143, or 148, which comprises an amino acid sequence of SEQ ID NO:4.
152. A construct according to any of paragraphs 99 to 130, 139, 146, 147, or 149 which comprises an amino acid sequence of SEQ ID NO: 16.
153. A construct according to any of paragraphs 99 to 128, 133, 134, 141, 144, 145, or 150, which comprises an amino acid sequence of SEQ ID NO:17.
154. An isolated nucleic acid molecule encoding a construct according to any preceding paragraph, or an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with the nucleic acid molecule over its entire length, or the complement thereof.
155. An isolated nucleic acid molecule according to paragraph 154, comprising a nucleotide sequence of SEQ ID NO:3, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 3 over its entire length, or the complement thereof.
156. An isolated nucleic acid molecule according to paragraph 154, comprising a nucleotide sequence of SEQ ID NO:5, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 5 over its entire length, or the complement thereof.
157. A vector comprising a nucleic acid molecule according to any of paragraphs 154 to 156.
158. A vector according to paragraph 157, which further comprises a promoter operably linked to the nucleic acid.
159. A vector according to paragraph 158, wherein the promoter is for expression of a polypeptide encoded by the nucleic acid in a protein expression cell line.
160. A vector according to paragraph 158 or 159, wherein the promoter is for expression of a polypeptide encoded by the nucleic acid in mammalian cells.
161. A vector according to paragraph 158 or 159, wherein the promoter is for expression of a polypeptide encoded by the nucleic acid in bacterial cells.
162. A vector according to paragraph any of paragraphs 157 to 160, wherein the vector is a pcDNA3 vector.
163. A vector according to any of paragraphs 157 to 159, and 161, wherein the vector is a pBAD vector.
164. A vector according to paragraph 158 or 159, wherein the promoter is for expression of a polypeptide encoded by the nucleic acid in yeast or insect cells.
165. A vector according to any of paragraphs 157 to 164, wherein the vector is a protein expression vector.
166. A vector according to paragraph 165, which is a DNA protein expression vector.
167. An isolated cell comprising a vector of any of paragraphs 157 to 166.
168. A pharmaceutical composition comprising a construct according to any of paragraphs 1 to 153, and a pharmaceutically acceptable carrier, excipient, or diluent.
169. A pharmaceutical composition according to paragraph 168, comprising a polypeptide which comprises an amino acid sequence of SEQ ID NO:4.
170. A pharmaceutical composition according to paragraph 168, comprising a polypeptide which comprises an amino acid sequence of SEQ ID NO:16.
171. A pharmaceutical composition according to paragraph 168, comprising a polypeptide which comprises an amino acid sequence of SEQ ID NO: 17.
172. A pharmaceutical composition according to paragraph 168, comprising a nucleic acid molecule according to any of paragraphs 154 to 156, and a pharmaceutically acceptable carrier, excipient, or diluent.
173. A pharmaceutical composition according to paragraph 172, comprising a nucleic acid molecule encoding a polypeptide which comprises an amino acid sequence of SEQ ID NO:4, or the complement thereof.
174. A pharmaceutical composition according to paragraph 172, comprising a nucleic acid molecule encoding a polypeptide which comprises an amino acid sequence of SEQ ID NO: 16, or the complement thereof.
175. A pharmaceutical composition according to paragraph 172, comprising a nucleic acid molecule encoding a polypeptide which comprises an amino acid sequence of SEQ ID NO: 17, or the complement thereof.
176. A pharmaceutical composition according to paragraph 172, comprising a nucleic acid molecule comprising a nucleotide sequence of SEQ ID NO:5.
177. A pharmaceutical composition according to paragraph, comprising a vector according to any of paragraphs 157 to 166, and a pharmaceutically acceptable carrier, excipient, or diluent.
178. A method for determining the IDH1/2 mutational status of a biological sample obtained from a subject, wherein the method comprises detecting for D-2-HG in the sample using a construct according to any of paragraphs 1 to 153.
179. A construct according to any of paragraphs 1 to 153, for use in determining the IDH1/2 mutational status in a tissue or cell of a subject in vivo, wherein determining the IDH1/2 mutational status of the tissue or cell comprises detecting for D-2-HG in the sample.
180. Use of a construct according to any of paragraphs 1 to 153, in the manufacture of a medicament for determining the IDH1/2 mutational status in a tissue or cell of a subject in vivo, wherein determining the IDH1/2 mutational status of the sample comprises detecting for D-2-HG in the sample.
181. A method according to paragraph 178, a construct according to paragraph 179, or use according to paragraph 180, wherein detecting for D-2-HG in the sample comprises determining the relative difference in fluorescence intensities between the first fluorescent reporter and the second fluorescent reporter, or between the second fluorescent reporter and the first fluorescent reporter.
182. A method, construct, or use according to paragraph 181, wherein the relative difference in fluorescence is a FRET ratio.
183. A method, construct, or use according to paragraph 182, wherein the FRET ratio is used to quantify D-2-HG in the biological sample obtained from a subject.
184. A method, construct, or use, according to paragraph 182 or 183, wherein a FRET ratio of ≥1.7 determines the presence of an IDH1/2 mutation.
185. A method according to paragraph 178, or 181 to 184, a construct according to paragraph 179 or 181 to 184, or use according to paragraph 180 to 184, wherein determining the IDH1/2 mutational status of a biological sample obtained from a subject is performed before, with, or after treatment with a medicament.
186. A method, construct, or use according to paragraph 185, wherein the medicament is an IDH1 inhibitor.
187. A method for monitoring a change in D-2-HG levels in a subject, which comprises:
   (i) obtaining a biological sample from a subject;
   (ii) detecting for D-2-HG in the sample by determining the relative difference in fluorescence intensities between the first fluorescent reporter and the second fluorescent reporter, or between the second fluorescent reporter and the first fluorescent reporter, in the biological sample using a construct according to any of paragraphs 1 to 153;
   (iii) repeating steps (i) and (ii) after a period of time; and
   (iv) determining whether the relative difference in fluorescence intensities between the first fluorescent reporter and the second fluorescent reporter, or between the second fluorescent reporter and the first fluorescent reporter, has changed.
188. A construct according to any of paragraphs 1 to 153 for use in monitoring a change in D-2-HG levels in a subject in vivo, wherein the monitoring of a change in D-2-HG levels comprises:
   (i) detecting D-2-HG by determining the relative difference in fluorescence intensities between the first fluorescent reporter and the second fluorescent reporter, or between the second fluorescent reporter and the first fluorescent reporter, in the biological sample using a construct according to any of paragraphs 1 to 153;
   (ii) repeating step (i) after a period of time; and
   (iii) determining whether the relative difference in fluorescence intensities between the first fluorescent reporter and the second fluorescent reporter, or between the second fluorescent reporter and the first fluorescent reporter, has changed.
189. Use of a construct according to any of paragraphs 1 to 153 in a method of manufacture of a medicament for monitoring a change in D-2-HG levels in a subject in vivo, wherein the monitoring of a change in D-2-HG levels comprises:
   (i) detecting for D-2-HG in the sample by determining the relative difference in fluorescence intensities between the first fluorescent reporter and the second fluorescent reporter, or between the second fluorescent reporter and the first fluorescent reporter, in the biological sample using a construct according to any of paragraphs 1 to 153;
   (ii) repeating step (i) after a period of time; and
   (iii) determining whether the relative difference in fluorescence intensities between the first fluorescent reporter and the second fluorescent reporter, or between the second fluorescent reporter and the first fluorescent reporter, has changed.
190. A method according to paragraph 187, construct according to paragraph 188, or use according to paragraph 189, wherein the relative difference in fluorescence is a FRET ratio.
191. A method, construct, or use according to paragraph 190, wherein the FRET ratio is used to quantify D-2-HG in the biological sample obtained from a subject.
192. A method, construct, or use, according to paragraph 190 or 191, wherein a FRET ratio of ≥1.7 determines the presence of an IDH1/2 mutation.
193. A method according to paragraph 187, or 190 to 192, a construct according to paragraph 188 or 190 to 192, or use according to paragraph 189 to 192, wherein monitoring a change in D-2-HG levels in a subject is performed before, with, or after treatment with a medicament.
194. A method, construct, or use according to paragraph 193, wherein the medicament is an IDH1 inhibitor.
195. A method according to any of paragraphs 178 to 187, or 190 to 194, a construct according to any of paragraphs 179 to 186, 188, or 190 to 194, or use according to any of paragraphs 180 to 186, or 189 to 194, wherein the biological sample is a cell culture, serum, tumour, plasma, urine, cerebrospinal fluid, or artificial cerebrospinal fluid sample.
196. A method, construct, or use according to paragraph 195, wherein the tumour sample is a glioma tumour sample.
197. A method, construct, or use, according to paragraph 196, wherein the tumour sample is a tumour lysates sample.
198. A method, construct, or use according to paragraph 195, wherein the cell culture sample is a cell culture supernatant sample.
199. A method according to any of paragraphs 178 to 187, or 190 to 198, a construct according to any of paragraphs 179 to 186, 188, or 190 to 198, or use according to any of paragraphs 180 to 186, or 189 to 198, wherein the D-2-HG is detected for in the cytoplasm, mitochondria, or nucleus of the cell.
200. A method according to any of paragraphs 178 to 187, or 190 to 199, a construct according to any of paragraphs 179 to 186, 188, or 190 to 199, or use according to any of paragraphs 180 to 186, or 189 to 199, wherein fluorescence intensities from the first and/or second fluorescent reporter are detected using a fluorescence microscope.
201. A method of analysing D-2-HG in a biological sample obtained from a subject, comprising detecting for D-2-HG in the cytoplasm or subcellular compartments of a cell in the biological sample using a construct which comprises a DhdR transcription factor linked to a first reporter, preferably a first fluorescent reporter, and a second reporter, preferably a second fluorescent reporter.
202. A construct for use in analysing D-2-HG in a cell of a subject in vivo, wherein the use comprises detecting for D-2-HG in the cytoplasm or subcellular compartments of the cell, wherein the construct comprises a DhdR transcription factor linked to a first reporter, preferably a first fluorescent reporter, and a second reporter, preferably a second fluorescent reporter.
203. Use of a construct in the manufacture of a medicament for analysing D-2-HG in a cell of a subject in vivo, wherein the construct comprises a DhdR transcription factor linked to a first reporter, preferably a first fluorescent reporter, and a second reporter, preferably a second fluorescent reporter, and wherein the use comprises detecting for D-2-HG in the cytoplasm or subcellular compartments of the cell.
204. A method according to paragraph 201, a construct according to paragraph 202, or use according to paragraph 203, where the construct comprises a construct according to any of paragraphs 1 to 153.
205. A method according to paragraph 201 or 204, a construct according to paragraph 202 or 204, or use according to paragraph 203 or 204, wherein detecting for D-2-HG in the cytoplasm or subcellular compartments of the cell comprises determining the relative difference in fluorescence intensities between the first fluorescent reporter and the second fluorescent reporter, or between the second fluorescent reporter and the first fluorescent reporter.
206. A method, construct, or use according to paragraph 205, wherein the relative difference in fluorescence is a FRET ratio.
207. A method, construct, or use according to paragraph 206, wherein the FRET ratio is used to quantify D-2-HG in the biological sample obtained from a subject.
208. A method, construct, or use according to paragraph 206 or 207, wherein a FRET ratio of ≥1.7 determines the presence of an IDH1/2 mutation.
209. A method according to paragraph 201 or 204 to 208, a construct according to paragraph 202 or 204 to 208, or use according to paragraph 203 or 204 to 208, wherein detecting for the D-2-HG in the cytoplasm or subcellular compartments of the cell in the biological sample determines the subcellular distribution of D-2-HG.
210. A method, construct, or use according to paragraph 209, wherein the subcellular location is the mitochondria, cytoplasm, or the nucleus.
211. A method, construct, or use according to paragraph 210, wherein the construct comprises a nuclear localisation signal.
212. A method, construct, or use according to paragraph 210, wherein the construct comprises a mitochondria localisation signal.
213. A method, construct, or use according to paragraph 210, wherein the fusion protein comprises a nuclear export signal.

### BRIEF DESCRIPTION OF THE DRAWINGS

Embodiments of the invention are described herein with reference to the drawings described as hereinafter. The patent application drawing file contains at least one drawing executed in color. Copies of this patent or patent application publication with color drawing(s) will be provided by the Office upon request and payment of the necessary fee.

FIGS. 1A-1C show the design and optimization of a genetically encoded fluorescent sensor of D-2-hydroxyglutarate (D-2-HG), wherein:

FIG. 1A is a schematic representation of a FRET-based D-2-HG sensor;

FIG. 1B is a graph showing representative D-2-HG binding curves for the parent FRET sensor and three sensor variants, wherein variant 2 is shown at the top of the binding curve, with variants 1 and 3 below; and

FIG. 1C is a graph showing a comparison of the dynamic range (Rmax/Rmin) of the parent sensor and three sensor variants.

FIGS. 2A-2D show in vitro characterization of purified D2HGlo, wherein:

FIG. 2A is a diagram of the Citric Acid Cycle, highlighting key intermediates;

FIG. 2B is a graph showing the selectivity of D2HGlo for D-2-HG compared with seven Citric Acid Cycle intermediates (citrate, isocitrate, α-ketoglutarate, succinate, fumarate, oxaloacetate and malate);

FIG. 2C is a graph showing the influence of pH on in vitro fluorescence measurements; and

FIG. 2D is a graph showing the impact of temperature on in vitro fluorescence measurements.

FIG. 3A-3E show in vitro characterization of purified D2HGlo, wherein:

FIG. 3A shows a top diagram illustrating how IDH1-R132H catalyzes the conversion of α-ketoglutarate to D-2-hydroxyglutarate and a bottom diagram of the Citric Acid Cycle, highlighting key intermediates;

FIG. 3B is a graph of representative in vitro D-2-HG and L-2-HG binding curves for purified D2HGlo;

FIG. 3C are comparative graphs showing the percent increase in FRET ratio above baseline levels in response to D-2-HG and L-2-HG at 1 μM (left graph) and 10 μM (right graph);

FIG. 3D is a graph showing the selectivity of D2HGlo for D-2-HG compared with six Citric Acid Cycle intermediates (citrate, isocitrate, α-ketoglutarate, succinate, oxaloacetate and malate); and

FIG. 3E is a graph showing the influence of pH on in vitro fluorescence measurements.

FIGS. 4A-4F show in vitro characterization of the response of D2HGlo to L-2-HG, wherein:

FIG. 4A is a diagram showing how IDH1-R132H catalyzes the conversion of α-ketoglutarate to D-2-HG;

FIG. 4B is a graph showing representative in vitro D-2-HG and L-2-HG binding curves for purified D2HGlo. FIG. 4C is a comparative graph showing the percent increase in FRET ratio above baseline levels in response to D-2-HG and L-2-HG at 1 μM (left) and 10 μM (right);

FIG. 4D are comparative graphs showing representative in vitro D-2-HG binding curves for purified D2HGlo in a background of low concentrations of L-2-HG (1.5-5.5 μM, top) or high background concentrations of L-2-HG (15-55 μM, bottom);

FIG. 4E is a graph showing the in vitro dynamic range of D2HGlo determined from three independent D-2-HG titrations in the presence of background L-2-HG ranging from 1.5-55 μM; and

FIG. 4F is a graph showing the Kd′ of D2HGlo for D-2-HG from three independent experiments performed in the presence of L-2-HG at concentrations ranging from 1.5-55μM.

FIGS. 5A-5F show imaging intracellular D-2-HG in HeLa cells using D2HGlo, wherein:

FIG. 5A is a representative image of HeLa cells expressing Cyto-D2HGlo;

FIG. 5B is ratiometric FRET image of HeLa cells expressing Cyto-D2HGlo before and after addition of 1 mM D-2-HG (top) or 1 mM L-2-HG (bottom);

FIG. 5C is a graph showing time-lapse imaging of HeLa cells expressing Cyto-D2HGlo following exposure to increasing concentrations of D-2-HG;

FIG. 5D is a graph shoing time-lapse imaging of HeLa cells expressing Cyto-D2HGlo following exposure to increasing concentrations of L-2-HG. Data is presented the same as in panel C;

FIG. 5E is a graph showing HeLa cells expressing Cyto-D2HGlo that showed a concentration-dependent increase in the maximum FRET ratio that could be achieved with either D-2-HG and L-2-HG; and

FIG. 5F is a graph showing HeLa cells expressing Cyto-D2HGlo that showed a concentration-dependent increase in the maximum FRET ratio that could be achieved with either D-2-HG and L-2-HG.

FIG. 6A-6C shows subcellular targeting of the D2HGlo sensor that reveals that D-2-HG levels are elevated in the cytosol, nucleus and mitochondria of IDH1-R132H mutant U87 glioma cells, wherein:

FIG. 6A shows on the left, a representative image of a wild-type IDH1 U87MG cell expressing Cyto-D2HGlo, and on the right, a comparison graph of the average FRET ratio in wild-type IDH1 and IDH1-R132H mutant U87MG cells expressing Cyto-D2HGlo;

FIG. 6B shows on the left, a representative image of a wild-type IDH1 U87 clel cell expressing Nuc-D2HGlo, and on the right, a comparison graph of the average FRET ratio in wild-type IDH1 and IDH1-R132H mutant U87 cells expressing Nuc-D2HGlo; and

FIG. 6C shows on the left, a representative image of a wild-type IDH1 U87 cell expressing Mito-D2HGlo, and on the rights, a comparison graph of the average FRET ratio in wild-type IDH1 and IDH1-R132H mutant U87 cells expressing Mito-D2HGlo.

FIGS. 7A-7D shows side-by-side analysis of real-time changes in D-2-HG levels in living cells and the D-2-HG concentration in cell supernatants, wherein:

FIG. 7A is a graph showing IDH1-wildtype or IDH1-R132H mutant U87MG cells expressing Cyto-D2HGlo, which were treated with 10 mM 2-Deoxy-D-glucose (2-DG) and the FRET ratio was monitored for approximately 5.5 minutes;

FIG. 7B is a graph showing IDH1-wildtype or IDH1-R132H mutant U87MG cells, which were treated with 2-DG (10 mM) for 48 hours after which the concentration of D-2-HG was determined in culture supernatants;

FIG. 7C is a graph showing IDH1-wildtype or IDH1-R132H mutant U87MG cells expressing Cyto-D2HGlo, which were treated with 1 μM Compound 968 (glutaminase C inhibitor) and the FRET ratio was monitored for approximately 5.5 minutes; and

FIG. 7D is a graph showing IDH1-wildtype or IDH1-R132H mutant U87MG cells, which were treated with Compound 968 (1 μM) for 48 hours after which the level of D-2-HG was assessed in culture supernatants.

FIG. 8 shows that D2HGlo can be used a diagnostic tool to monitor D-2-HG levels in human biological fluids, wherein:

FIG. 8A is a graph showing a comparison of the D-2-HG concentration in supernatants collected form wild-type IDH1 and IDH1-R132H mutant U87MG cells;

FIG. 8B is a graph showing titration of purified D2HGlo with increasing concentrations of D-2-HG (100 nM-1 mM) spiked into human serum;

FIG. 8C is a graph showing average FRET ratio of D2HGlo in buffer, unspiked human serum and serum containing 1 μM, 3.2 μM or 10 μM D-2-HG;

FIG. 8D is a graph showing titration of purified D2HGlo with increasing concentrations of D-2-HG (100 nM-1 mM) spiked into human urine;

FIG. 8E is a graph showing average FRET ratio of D2HGlo in buffer, unspiked human urine and urine containing 1 μM, 3.2 μM or 10 μM D-2-HG;

FIG. 8F is a graph showing titration of purified D2HGlo with increasing concentrations of D-2-HG (100 nM-1 mM) spiked into aCSF; and

FIG. 8G is a graph showing average FRET ratio of D2HGlo in buffer, unspiked aCSF and aCSF containing 1 μM, 3.2 μM or 10 μM D-2-HG.

FIG. 9 shows that D2HGlo accurately predicts the IDH1 mutational status of brain tumor samples from human patients, wherein:

FIG. 9A is a graph wherein the D2HGlo FRET ratio is shown for twenty brain tumor samples;

FIG. 9B is a graph showing the average FRET ratio for wild-type IDH1 compared with IDH1-R132H mutants;

FIG. 9C is a table showing diagnosis, pathology report and the raw FRET value for all twenty brain tumor samples; and

FIG. 9D is a table, like FIG. 9C but showing the correlating concentration of D-2-HG in μM.

FIG. 10 is a graph showing the emission spectrum of purified D2HGlo.

FIG. 11A is a graph shows the limit of detection of D2HGlo wherein purified D2HGlo was exposed to concentrations of D-2-HG, ranging from 100 nM to 500 nM, before the FRET ratio was measured.

FIG. 11B is a further graph of the limit of detection wherein purified D2HGlo was exposed to concentrations of D-2-HG ranging from 50 nM to 500 nM, and the FRET ratio was collected at each concentration.

FIGS. 12A-121 are graphs showing that D2HGlo does not respond to Citric Acid Cycle intermediates, pyruvate, lactate and glutamate, wherein a purified sensor was titrated with increasing concentrations of:

FIG. 12A Citrate;

FIG. 12B Isocitrate;

FIG. 12C α-ketoglutarate;

FIG. 12D Succinate;

FIG. 12E Oxaloacetate;

FIG. 12F Malate;

FIG. 12G Pyruvate;

FIG. 12H Lactate; and

FIG. 12I Glutamate.

FIG. 13 is a graph showing D2HGlo function is unchanged by L-2-HG. D2HGlo was exposed to D-2-HG in the absence (blue) and presence (red) of 1 μM L-2-HG.

FIG. 14A-14B show viability of glioma cell lines decreases after exposure to elevated concentrations of membrane-permeable D-2-HG and L-2-HG, wherein:

FIG. 14A is a graph of showing cell viability of IDH1-WT U87MG cells, where IDH1-R132H mutant U87MG cells and human fetal glial SVG cells was measured using CellTiter-Glo® Luminescent Cell Viability Assay and cells were treated for 24 h with varying concentrations of octyl-D-2-HG (1.9 M-4 mM); and

FIG. 14B is a graph for the same set of experiments performed with octyl-L-2-HG.

FIG. 15 is a graph showing IDH1-R132H mutant U87MG cells expressing Cyto-D2HGlo do not respond to acute treatment with AG-120 wherein cells expressing Cyto-D2HGlo (48 h post-transfection) were treated with AG-120 (10 μM) and the FRET ratio was monitored for approximately 15 minutes after drug addition. Each line represents a single cell treated with AG-120.

FIG. 16A shows expression of Nuc-D2HGlo and Mito-D2HGlo in HeLa cells. HeLa cells were transfected with Nuc-D2HGlo or Mito-D2HGlo and imaged on a confocal microscope at 48 hours post-transfected.

FIG. 16B shows colocalization of Mito-D2HGlo with Mito Tracker DeepRed. HeLa cells were transfected with Mito-D2HGlo. At 48 hours post-transfected, cells were exposed to Mito Tracker for 10 min prior to fluorescence imaging. Separate images of Mito-D2HGlo and Mito Tracker DeepRed are shown. An overlay of both channels is shown on the right.

FIGS. 17A and 17B show a comparison of FRET ratio in wild-type IDH1 and IDH1-R132H mutant U87 cells among three targeted D2HGlo sensors, wherein:

FIG. 17A is a graph showing a comparison of the D2HGlo FRET ratio in the cytosol, nucleus and mitochondria of wild-type IDH1 U87 cells. The average±standard deviation is shown for n=59 cells obtained from four independent experiments (cytosol), n=59 cells obtained from four independent experiments (nucleus) and n=47 cells obtained from three independent experiments (mitochondria).

FIG. 17B is a graph showing a comparison of the D2HGlo FRET ratio in the cytosol, nucleus and mitochondria of IDH1-R132H mutant U87 cells. The average±standard deviation is shown for n=61 cells obtained from four independent experiments (cytosol), n=58 cells obtained from four independent experiments (nucleus) and n=48 cells obtained from three independent experiments (mitochondria). (A&B) Statistical analysis was performed using a one-way ANOVA test with post hoc Tukey (****, P<0.0001; *, P<0.05).

FIG. 18A-B show that EDTA abolishes D2HGlo FRET response, wherein:

FIG. 18A shows representative trace of D-2-HG dose-response curve with and without EDTA (1 mM) included in the titration buffer.

FIG. 18B shows direct comparison of percent increase in FRET ratio elicited by 10 μM D-2-HG (left) and 100 μM D-2-HG (right) in the absence and presence of 1 mM EDTA. The average±standard deviation for each condition is shown for five independent experiments. Statistical analysis was performed using an unpaired t-test (****, P<0.0001).

FIG. 19 shows expression of targeted D2HGlo sensors (Cyto-D2HGlo, Nuc-D2HGlo, Mito-D2HGlo) in HeLa cells (FIG. 19A) and IDH1-R132H mutant U87MG cells (FIG. 19B). Scale bar is 50 μm.

### DETAILED DESCRIPTION

The current application describes the development, optimization and applications of D2HGlo. Embodiments of the invention are now described, by way of example only, with reference to the accompanying drawings, in which:

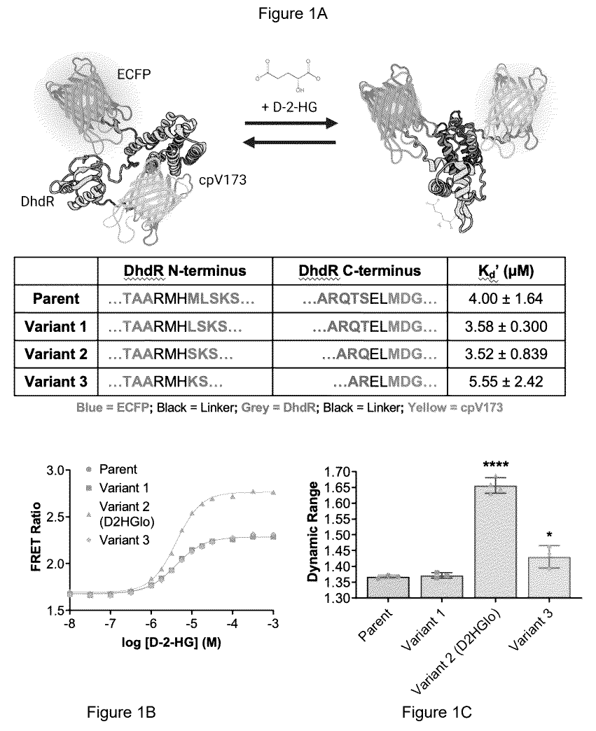

FIGS. 1A-1C show the design and optimization of a genetically encoded fluorescent sensor of D-2-hydroxyglutarate (D-2-HG); (A) Schematic representation of a FRET-based D-2-HG sensor. The transcription factor, DhdR, is flanked by two fluorescent proteins (ECFP and cpVenus173) that act as a FRET pair. The structure of DhdR was predicted using AlphaFold. The FRET sensor model was generated in BioRender. The amino acid sequences at the N-terminus and C-terminus of DhdR are shown for the parent sensor and three sensor variants, along with the Kd′ values for each construct. From left to right, the amino acid sequence of the DhdR N-terminus comprises the following amino components (shown as differing greyscale shading on the figure): ECFP; linker; DhdR. From left to right, the amino acid sequence of the DhdR C-terminus comprises the following amino components (shown as differing greyscale shading on the figure): DhdR; linker; cpV173; (B) Representative D-2-HG binding curves for the parent FRET sensor and three sensor variants. Variant 2 is shown at the top of the binding curve, with variants 1 and 3 below; (C) Comparison of the dynamic range (Rmax/Rmin) of the parent sensor and three sensor variants. The average dynamic range±standard deviation is shown for 3-4 independent experiments. Statistical analysis was performed using a one-way ANOVA test with post hoc Tukey (****, P<0.0001 compared with Parent, Variant 1 and Variant 3; *, P<0.05 compared with Parent and Variant 1);

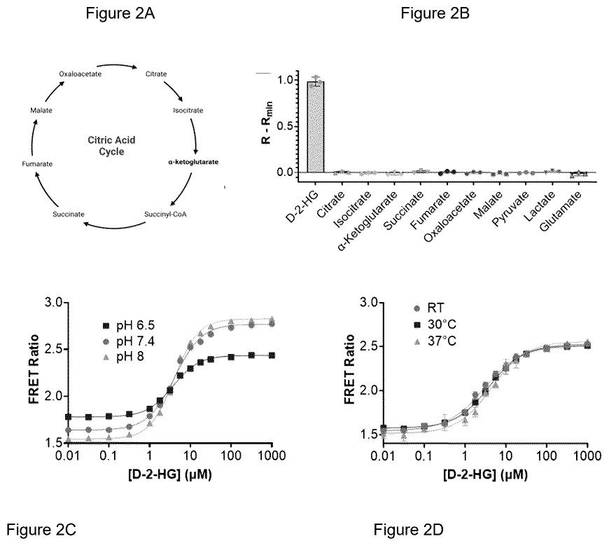

FIG. 2 shows in vitro characterization of purified D2HGlo. (A) Diagram of the Citric Acid Cycle, highlighting key intermediates. (B) Selectivity of D2HGlo for D-2-HG compared with seven Citric Acid Cycle intermediates (citrate, isocitrate, α-ketoglutarate, succinate, fumarate, oxaloacetate and malate). In addition, pyruvate, lactate and glutamate were tested. R-Rmin was calculated by subtracting the FRET ratio in the presence of 100 nM metabolite (Rmin) from the FRET ratio in the presence of 100 μM metabolite (R). The average±standard deviation for each condition is shown for three independent experiments. (C) The influence of pH on in vitro fluorescence measurements. Purified D2HGlo was diluted in three different buffers (pH 6.5, pH 7.4 and pH 8), and the representative binding curves are shown. (D) The impact of temperature on in vitro fluorescence measurements. D2HGlo was incubated at 30° C. or 37° C. in the presence of D-2-HG for 15 minutes prior to fluorescent measurements and the representative binding curves are shown.

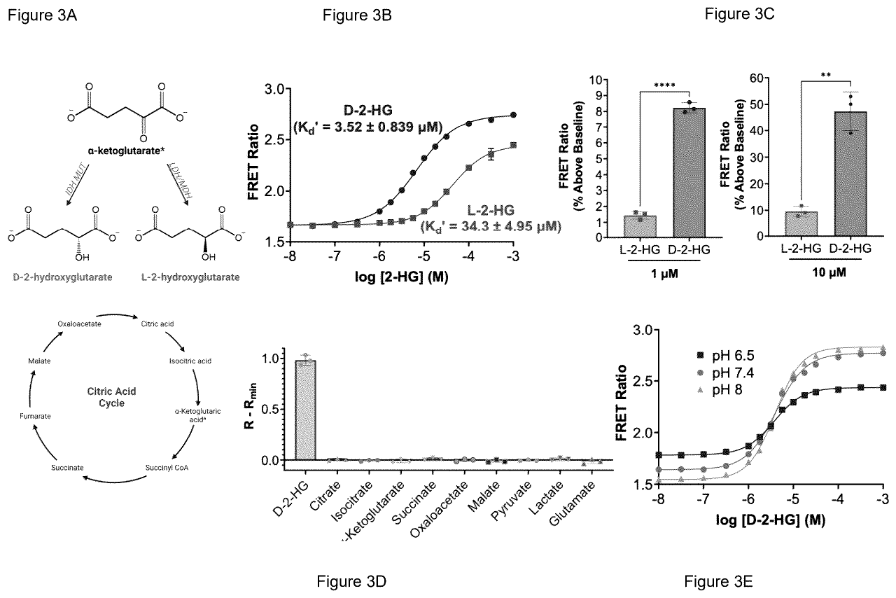

FIG. 3 shows in vitro characterization of purified D2HGlo. (A) Top: IDH1-R132H catalyzes the conversion of α-ketoglutarate to D-2-hydroxyglutarate. L-2-HG is produced by lactate dehydrogenase (LDH) and malate dehydrogenase (MDH). Bottom: Diagram of the Citric Acid Cycle, highlighting key intermediates. (B) Representative in vitro D-2-HG (top line) and L-2-HG (bottom line) binding curves for purified D2HGlo. The average Kd′ is shown for D-2-HG (blue) and L-2-HG (red) and represents three independent experiments. (C) The percent increase in FRET ratio above baseline levels in response to D-2-HG and L-2-HG at 1 μM (left) and 10 μM (right). The average±standard deviation for each condition is shown for three independent experiments. Statistical analysis was performed using an unpaired t-test (****, P<0.0001; **, P<0.01). (D) Selectivity of D2HGlo for D-2-HG compared with six Citric Acid Cycle intermediates (citrate, isocitrate, α-ketoglutarate, succinate, oxaloacetate and malate). In addition, pyruvate, lactate and glutamate were tested. R-Rmin was calculated by subtracting the FRET ratio at 100 nM metabolite (Rmin) from the FRET ratio at 100 μM metabolite (R). The average±standard deviation for each condition is shown for three independent experiments. (E) The influence of pH on in vitro fluorescence measurements. Purified D2HGlo was diluted in three different buffers (pH 6.5, pH 7.4 and pH 8), and the representative binding curves are shown;

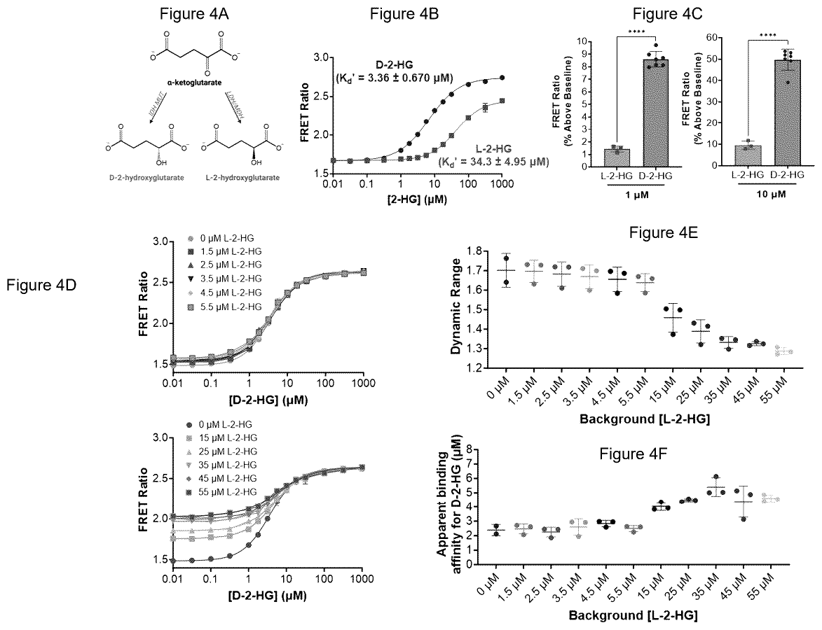

FIG. 4 shows in vitro characterization of the response of D2HGlo to L-2-HG. (A) IDH1-R132H catalyzes the conversion of α-ketoglutarate to D-2-HG. L-2-HG is produced by lactate dehydrogenase (LDH) and malate dehydrogenase (MDH). (B) Representative in vitro D-2-HG (top line) and L-2-HG (bottom line) binding curves for purified D2HGlo. The average K_{d}′±standard deviation is shown for D-2-HG (blue) and L-2-HG (red) and represents 3-7 independent experiments. (C) The percent increase in FRET ratio above baseline levels in response to D-2-HG and L-2-HG at 1 μM (left) and 10 μM (right). The average±standard deviation for each condition is shown for three independent experiments. Statistical analysis was performed using an unpaired t-test (****, P<0.0001). (D) Representative in vitro D-2-HG binding curves for purified D2HGlo in a background of low concentrations of L-2-HG (1.5-5.5 μM, top) or high background concentrations of L-2-HG (15-55 μM, bottom). (E) The in vitro dynamic range of D2HGlo determined from three independent D-2-HG titrations in the presence of background L-2-HG ranging from 1.5-55 μM. (F) The K_{d}′ of D2HGlo for D-2-HG from three independent experiments performed in the presence of L-2-HG at concentrations ranging from 1.5-55 μM.

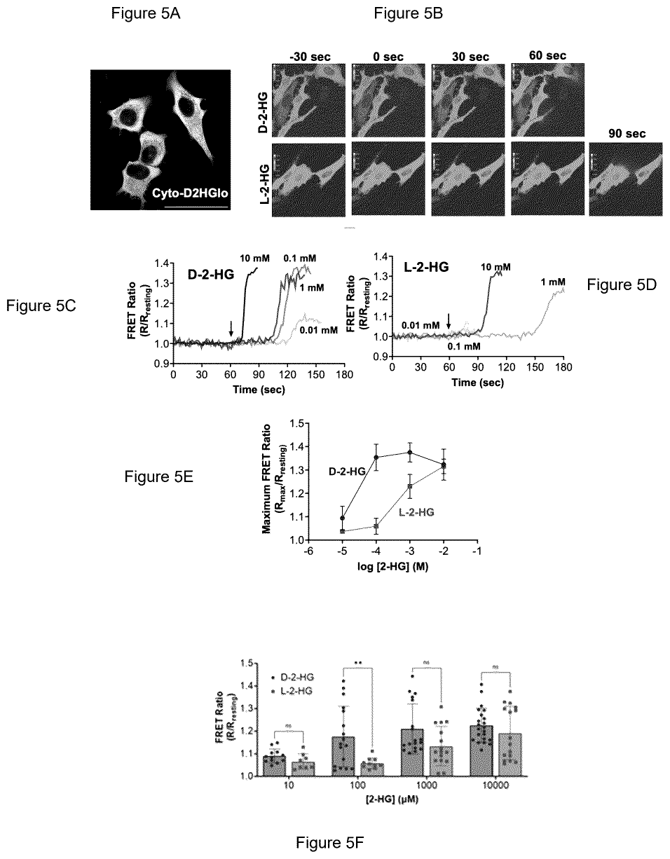

FIG. 5 shows imaging intracellular D-2-HG in HeLa cells using D2HGlo. (A) Representative image of HeLa cells expressing Cyto-D2HGlo. Scale bar is 50 μm. (B) Ratiometric FRET image of HeLa cells expressing Cyto-D2HGlo before and after addition of 1 mM D-2-HG (top) or 1 mM L-2-HG (bottom). Images are shown at 30 seconds before 2-HG addition (−30 sec), at the time of addition (0 sec) or for 30-90 sec following addition. (C) Time-lapse imaging of HeLa cells expressing Cyto-D2HGlo following exposure to increasing concentrations of D-2-HG. The FRET ratio was collected for one minute prior to the addition of digitonin (25 μM) and 0.01 mM, 0.1 mM, 1 mM or 10 mM D-2-HG. The FRET ratio (R) at each time point was normalized to the average FRET ratio recorded during the baseline measurements (Rresting). (D) Time-lapse imaging of HeLa cells expressing Cyto-D2HGlo following exposure to increasing concentrations of L-2-HG. Data is presented the same as in panel C. (E) HeLa cells expressing Cyto-D2HGlo showed a concentration-dependent increase in the maximum FRET ratio that could be achieved with either D-2-HG (top line) and L-2-HG (bottom line). Cells were treated with 25 μM digitonin and increasing concentrations of D-2-HG or L-2-HG, ranging from 0.01-10 mM. For D-2-HG, each data point represents n=3 cells from one independent experiment (0.01 mM D-2-HG), n=5 cells from two independent experiments (0.1 mM D-2-HG), n=5 cells from two independent experiments (1 mM D-2-HG) and n=5 cells from two independent experiments (10 mM D-2-HG). For L-2-HG, each data point represents n=4 cells from one independent experiment (0.01 mM L-2-HG), n=5 cells from two independent experiments (0.1 mM L-2-HG), n=6 cells from two independent experiments (1 mM L-2-HG) and n=7 cells from two independent experiments (10 mM L-2-HG). (F) HeLa cells expressing Cyto-D2HGlo showed a concentration-dependent increase in the maximum FRET ratio that could be achieved with either D-2-HG and L-2-HG. Cells were treated with 25 μM digitonin and increasing concentrations of D-2-HG or L-2-HG, ranging from 0.01-10 mM. For D-2-HG, the bar graph represents n=12 cells from four independent experiments (0.01 mM D-2-HG), n=17 cells from six independent experiments (0.1 mM D-2-HG), n=18 cells from six independent experiments (1 mM D-2-HG) and n=22 cells from eight independent experiments (10 mM D-2-HG). For L-2-HG, the bar graph represents n=8 cells from three independent experiments (0.01 mM L-2-HG), n=10 cells from five independent experiments (0.1 mM L-2-HG), n=16 cells from six independent experiments (1 mM L-2-HG) and n=16 cells from five independent experiments (10 mM L-2-HG). Statistical analysis was performed using a two-way ANOVA test with post hoc Tukey (**, P<0.01);

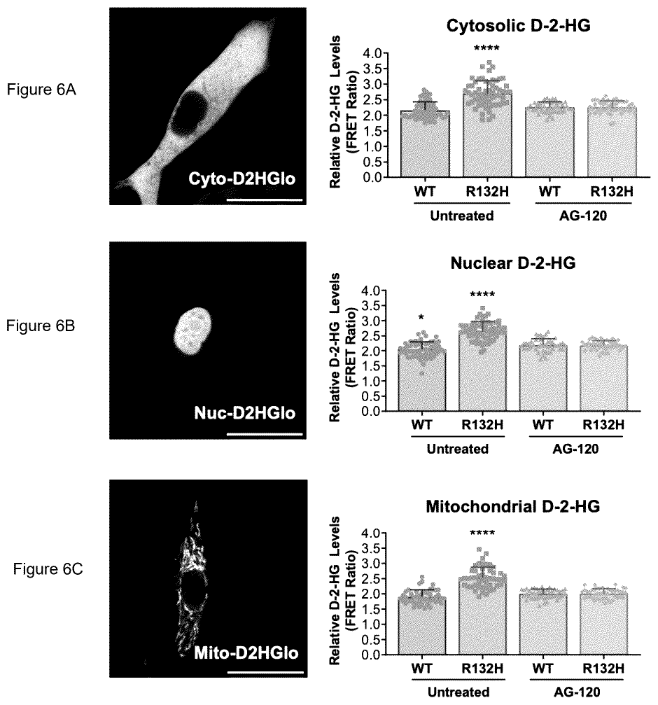

FIG. 6 shows subcellular targeting of the D2HGlo sensor reveals that D-2-HG levels are elevated in the cytosol, nucleus and mitochondria of IDH1-R132H mutant U87 glioma cells. (A) Left: Representative image of a wild-type IDH1 U87MG cell expressing Cyto-D2HGlo. Scale bar is 25 μm. Right: Comparison of the average FRET ratio in wild-type IDH1 and IDH1-R132H mutant U87MG cells expressing Cyto-D2HGlo. Each cell line was divided into two groups: untreated or treated with IDH1-R132H inhibitor AG-120 (3 μM) for 48 hours prior to collecting images. The average±standard deviation is shown for n=59 cells obtained from four independent experiments (untreated WT), n=48 cells obtained from three independent experiments (AG-120-treated WT), n=61 cells from four independent experiments (untreated R132H) and n=47 cells from three independent experiments (AG-120-treated R132H). (B) Left: Representative image of a wild-type IDH1 U87 clel cell expressing Nuc-D2HGlo. Scale bar is 25 μm. Right: Comparison of the average FRET ratio in wild-type IDH1 and IDH1-R132H mutant U87 cells expressing Nuc-D2HGlo. Same treatments as panel A. The average±standard deviation is shown for n=59 cells obtained from four independent experiments (untreated WT), n=45 cells obtained from three independent experiments (AG-120-treated WT), n=58 cells from four independent experiments (untreated R132H) and n=45 cells from three independent experiments (AG-120-treated R132H). (C) Left: Representative image of a wild-type IDH1 U87 cell expressing Mito-D2HGlo. Scale bar is 25 μm. Right: Comparison of the average FRET ratio in wild-type IDH1 and IDH1-R132H mutant U87 cells expressing Mito-D2HGlo. Same treatments as panel A. The average±standard deviation is shown for n=47 cells obtained from three independent experiments (untreated WT), n=45 cells obtained from three independent experiments (AG-120-treated WT), n=48 cells from three independent experiments (untreated R132H) and n=45 cells from three independent experiments (AG-120-treated R132H). (A-D) Statistical analysis was performed using a one-way ANOVA test with post hoc Tukey (****, P<0.0001 compared with untreated WT, AG-120-treated WT and AG-120-treated R132H; *, P<0.05 compared with AG-120-treated WT and AG-120-treated R132H);

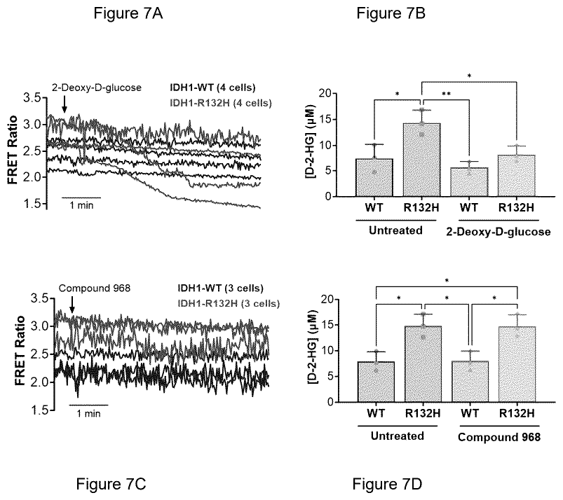

FIG. 7 shows side-by-side analysis of real-time changes in D-2-HG levels in living cells and the D-2-HG concentration in cell supernatants. (A) IDH1-wildtype (bottom lines) or IDH1-R132H mutant (top lines, and top lines which decrease after 2-DG is added)) U87MG cells expressing Cyto-D2HGlo were treated with 10 mM 2-Deoxy-D-glucose (2-DG) and the FRET ratio was monitored for approximately 5.5 minutes. Each line represents a single cell treated with 2-DG (n=4 cells for WT and R132H). (B) IDH1-wildtype or IDH1-R132H mutant U87MG cells were treated with 2-DG (10 mM) for 48 hours after which the concentration of D-2-HG was determined in culture supernatants. Statistical analysis was performed using a one-way ANOVA test with post hoc Tukey (**, P<0.01; *, P<0.05). (C) IDH1-wildtype (bottom lines) or IDH1-R132H mutant (top lines) U87MG cells expressing Cyto-D2HGlo were treated with 1 μM Compound 968 (glutaminase C inhibitor) and the FRET ratio was monitored for approximately 5.5 minutes. Each line represents a single cell treated with Compound 968 (n=3 cells for WT and R132H). (D) IDH1-wildtype or IDH1-R132H mutant U87MG cells were treated with Compound 968 (1 μM) for 48hours after which the level of D-2-HG was assessed in culture supernatants. Statistical analysis was performed using a one-way ANOVA test with post hoc Tukey (*, P<0.05);

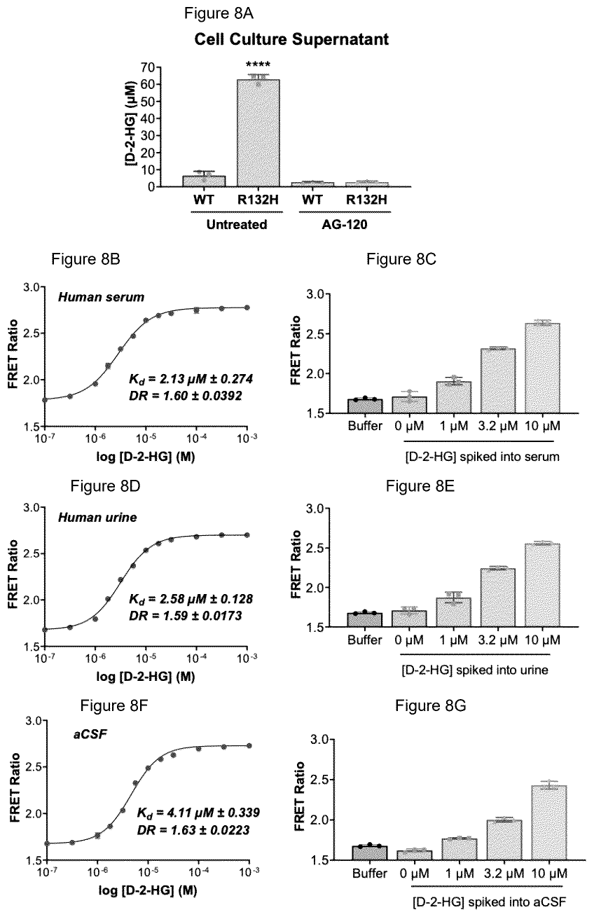

FIG. 8 shows D2HGlo can be used a diagnostic tool to monitor D-2-HG levels in human biological fluids. (A) Comparison of the D-2-HG concentration in supernatants collected form wild-type IDH1 and IDH1-R132H mutant U87MG cells. Both cell lines were left untreated or exposed to AG-120 (3 μM) for 48 hours prior to measurements. (B) Titration of purified D2HGlo with increasing concentrations of D-2-HG (100 nM-1 mM) spiked into human serum. The Kd′ and dynamic range for three independent replicates is shown in the plot. (C) Average FRET ratio of D2HGlo in buffer, unspiked human serum and serum containing 1 μM, 3.2 μM or 10 μM D-2-HG. (D) Titration of purified D2HGlo with increasing concentrations of D-2-HG (100 nM-1 mM) spiked into human urine. The Kd′ and dynamic range for three independent replicates is shown in the plot. (E) Average FRET ratio of D2HGlo in buffer, unspiked human urine and urine containing 1 μM, 3.2 μM or 10 μM D-2-HG. (F) Titration of purified D2HGlo with increasing concentrations of D-2-HG (100 nM-1 mM) spiked into aCSF. The Kd′ and dynamic range for three independent replicates is shown in the plot. (G) Average FRET ratio of D2HGlo in buffer, unspiked aCSF and aCSF containing 1 μM, 3.2 μM or 10 μM D-2-HG;

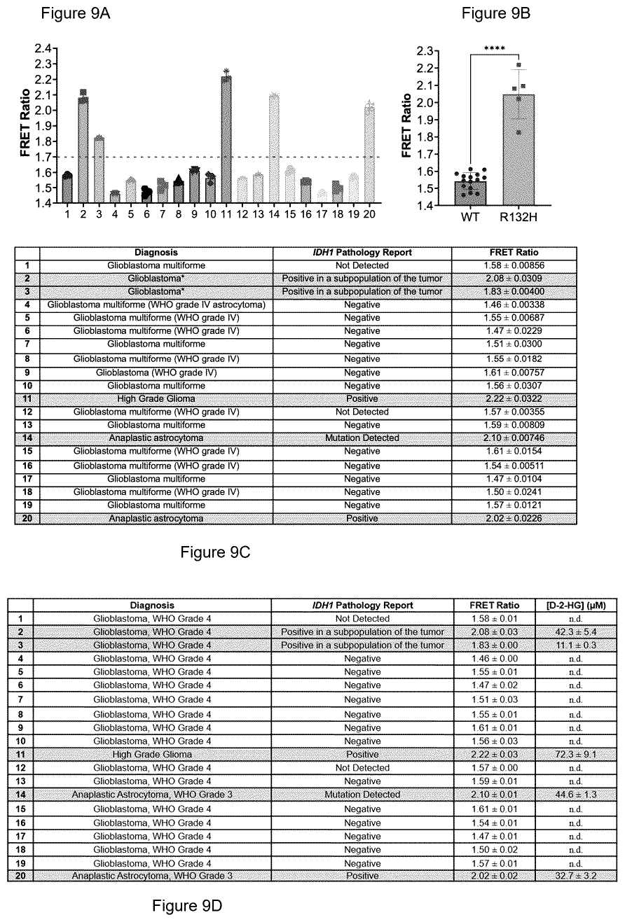

FIG. 9 shows that D2HGlo accurately predicts the IDH1 mutational status of brain tumor samples from human patients. (A) The D2HGlo FRET ratio is shown for twenty brain tumor samples. 5 samples were derived from IDH1-R132H mutant individuals, and 15 were derived from wildtype IDH1 samples. FRET ratio of 1.7 was used as a threshold to predict IDH1 mutational status. (B) The average FRET ratio for wild-type IDH1 compared with IDH1-R132H mutants. Statistical analysis was performed using an unpaired t-test (****, P<0.0001). (C) Table showing diagnosis, pathology report and the raw FRET value for all twenty brain tumor samples (n.d.=not detected using D2HGlo). This pathology report was provided prior to the release of the fifth edition of the World Health Organization (WHO) Classification of Tumors of the Central Nervous System (CNS5). While samples 2 and 3 are listed as glioblastoma, new classifications place gliomas with IDH mutations in a separate category from GBM. (D) Table as per (C), but showing the correlating concentration of D-2-HG in μM;

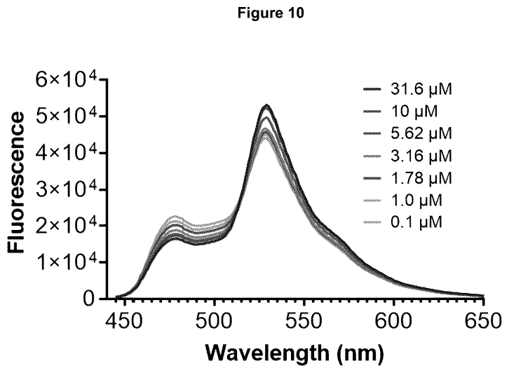

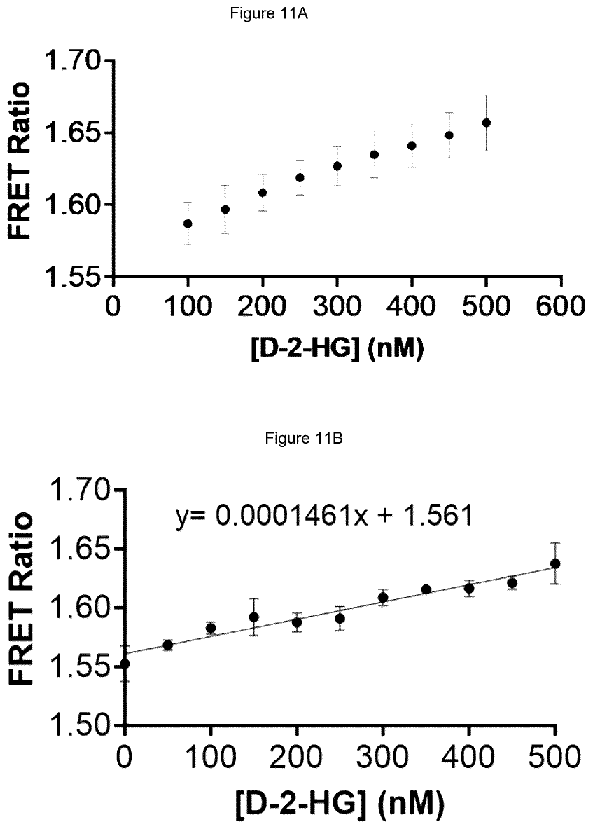

FIG. 10 shows the emission spectrum of purified D2HGlo. The purified sensor was exposed to D-2-HG at concentrations ranging from 0.1-31.6 μM. The FRET sensor was excited at 440 nm, and the emission spectrum window was collected between 450-650 nm. As the concentration of D-2-HG increased, the 482 nm peak corresponding with ECFP increased and the 531 nm peak increased. Thus, this supports a model in which D-2-HG binding DhdR results in increased FRET between the two fluorescent proteins;

FIG. 11 shows the limit of detection of D2HGlo. (A) Purified D2HGlo was exposed to concentrations of D-2-HG, ranging from 100 nM to 500 nM, before the FRET ratio was measured. (B) Purified D2HGlo was exposed to concentrations of D-2-HG ranging from 50 nM to 500 nM, and the FRET ratio was collected at each concentration. The LOD of D2HGlo was determined to be 308 nM. The limit of detection (LOD) was determined using the following equation: LOD=3o′/S, where o′ is the standard deviation of the FRET ratio measurements collected from the blank (no D-2-HG present) and S is the slope obtained from linear regression analysis;

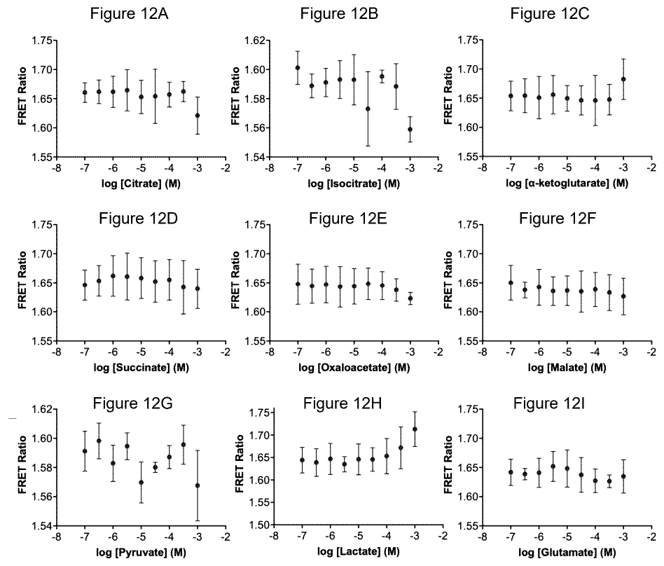

FIG. 12 shows that D2HGlo does not respond to Citric Acid Cycle intermediates, pyruvate, lactate and glutamate. Purified sensor was titrated with increasing concentrations of: (A) Citrate, (B) Isocitrate, (C) α-ketoglutarate, (D) Succinate, (E) Oxaloacetate, (F) Malate, (G) Pyruvate, (H) Lactate and (I) Glutamate. The FRET ratio is plotted against the log concentration of each metabolite. Each data point represents the average±standard deviation from three independent experiments;

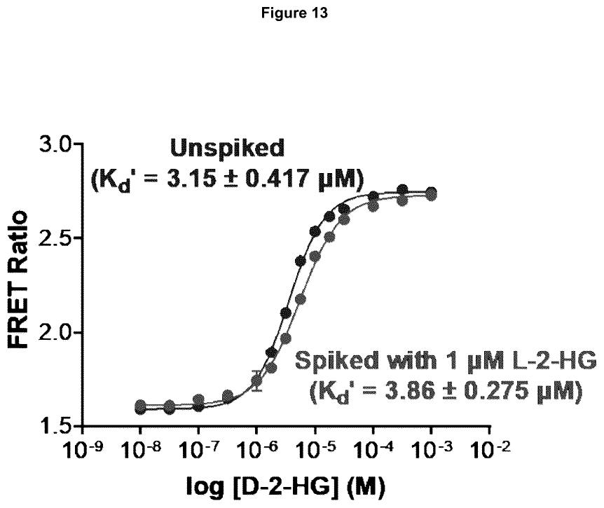

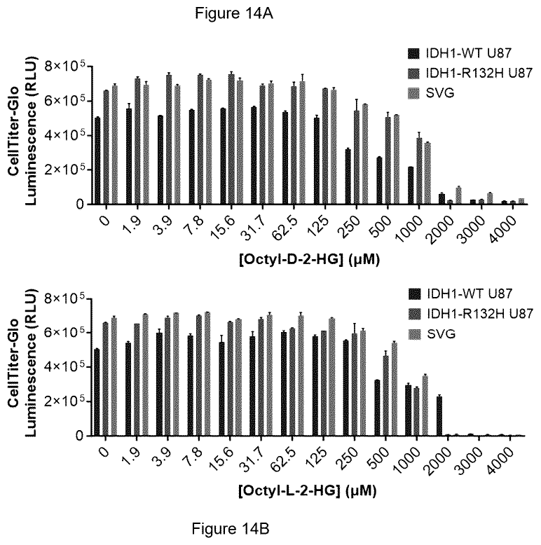

FIG. 13 shows D2HGlo function is unchanged by L-2-HG. D2HGlo was exposed to D-2-HG in the absence (blue; top line at 10⁻⁵M D-2HG) and presence (red; bottom line at 10⁻⁵M D-2HG) of 1 μM L-2-HG. Representative binding curves are shown for each condition. The average Kd′ is shown for D-2-HG (blue) and L-2-HG (red) and represents three independent experiments;

FIG. 14 shows viability of glioma cell lines decreases after exposure to elevated concentrations of membrane-permeable D-2-HG and L-2-HG. (A) Cell viability of IDH1-WT U87MG cells, IDH1-R132H mutant U87MG cells and human fetal glial SVG cells (shown in order from left to right at each Octyl-D-2-HG or Octyl-L-2-HG data point) was measured using CellTiter-Glo® Luminescent Cell Viability Assay. Cells were treated for 24h with varying concentrations of octyl-D-2-HG (1.9 μM-4 mM). (B) Same set of experiments performed with octyl-L-2-HG;

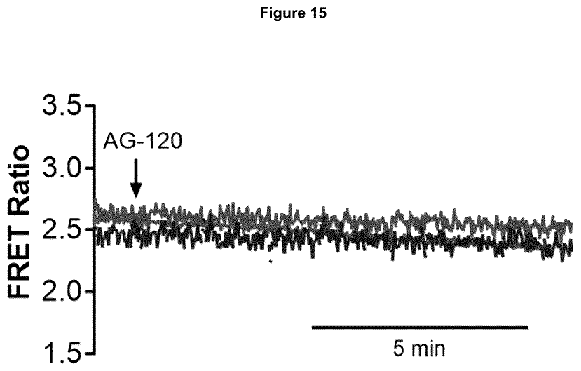

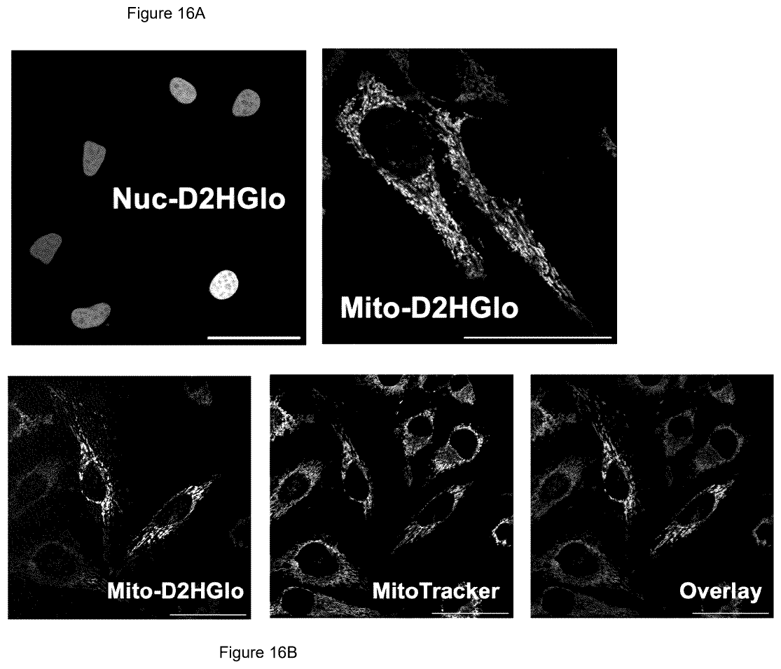

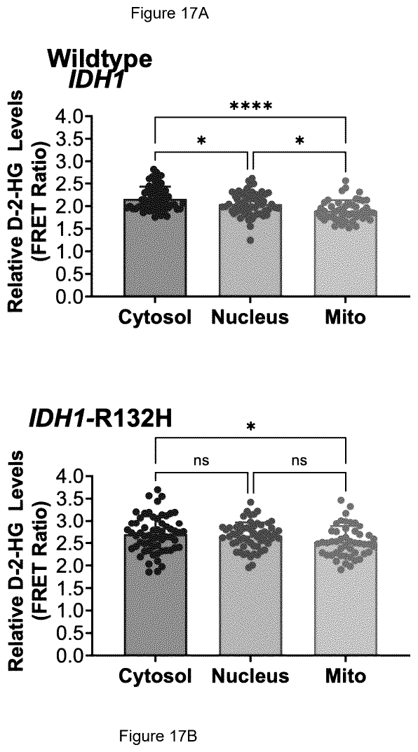

FIG. 15 shows IDH1-R132H mutant U87MG cells expressing Cyto-D2HGlo do not respond to acute treatment with AG-120. Cells expressing Cyto-D2HGlo (48h post-transfection) were treated with AG-120 (10 μM) and the FRET ratio was monitored for approximately 15 minutes after drug addition. Each line represents a single cell treated with AG-120;

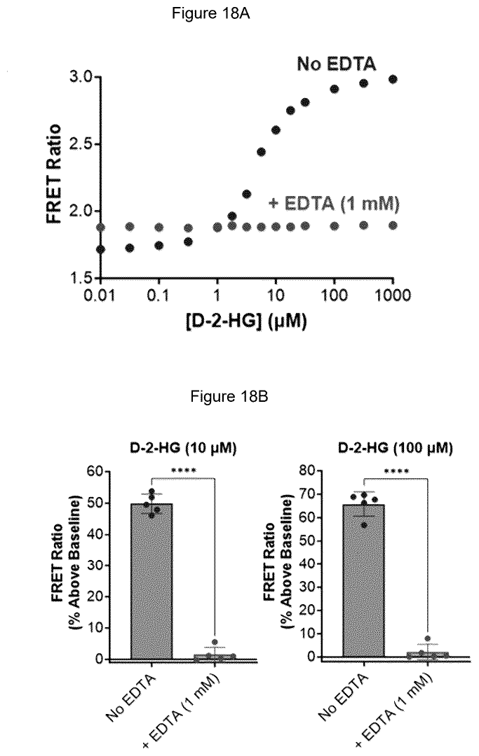

FIG. 16 shows (A) expression of Nuc-D2HGlo and Mito-D2HGlo in HeLa cells. HeLa cells were transfected with Nuc-D2HGlo or Mito-D2HGlo and imaged on a confocal microscope at 48 hours post-transfected. Scale bar is 50 μm. (B) Colocalization of Mito-D2HGlo with MitoTracker DeepRed. HeLa cells were transfected with Mito-D2HGlo. At 48 hours post-transfected, cells were exposed to Mito Tracker for 10 min prior to fluorescence imaging. Separate images of Mito-D2HGlo and MitoTracker DeepRed are shown. An overlay of both channels is shown on the right;

FIG. 17 shows comparison of FRET ratio in wild-type IDH1 and IDH1-R132H mutant U87 cells among three targeted D2HGlo sensors. (A) Comparison of the D2HGlo FRET ratio in the cytosol, nucleus and mitochondria of wild-type IDH1 U87 cells. The average±standard deviation is shown for n=59 cells obtained from four independent experiments (cytosol), n=59 cells obtained from four independent experiments (nucleus) and n=47 cells obtained from three independent experiments (mitochondria). (B) Comparison of the D2HGlo FRET ratio in the cytosol, nucleus and mitochondria of IDH1-R132H mutant U87 cells. The average±standard deviation is shown for n=61 cells obtained from four independent experiments (cytosol), n=58 cells obtained from four independent experiments (nucleus) and n=48 cells obtained from three independent experiments (mitochondria). (A&B) Statistical analysis was performed using a one-way ANOVA test with post hoc Tukey (****, P<0.0001; *, P<0.05);

FIG. 18 shows EDTA abolishes D2HGlo FRET response. (A) Representative trace of D-2-HG dose-response curve with and without EDTA (1 mM) included in the titration buffer. (B) Direct comparison of percent increase in FRET ratio elicited by 10 μM D-2-HG (left) and 100 μM D-2-HG (right) in the absence and presence of 1 mM EDTA. The average±standard deviation for each condition is shown for five independent experiments. Statistical analysis was performed using an unpaired t-test (****, P<0.0001); and

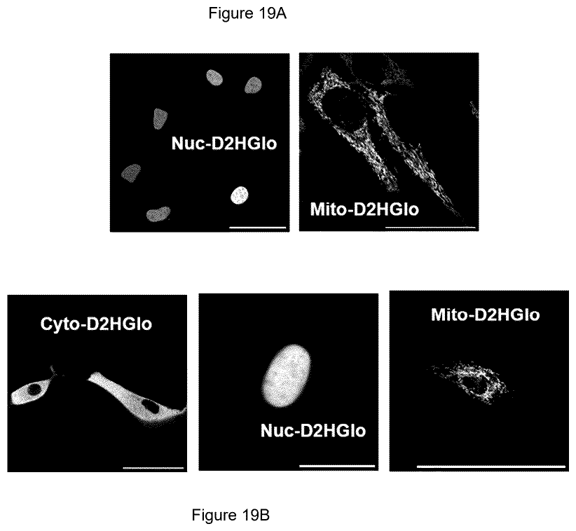

FIG. 19 shows expression of targeted D2HGlo sensors in HeLa cells and IDH1-R132H mutant U87MG cells. HeLa cells (A) and IDH1 mutant U87MG cells (B) were transfected with Cyto-D2HGlo, Nuc-D2HGlo, Mito-D2HGlo and imaged on a confocal microscope at 48 hours post-transfected. Scale bar is 50 μm.

### SEQ ID NO Table

1 Full length DhdR (amino acid sequence)
2 Variant 2 truncated DhdR (amino acid sequence)
3 Variant 2 truncated DhdR (nucleic acid sequence)
4 D2HGlo (amino acid sequence)
5 D2HGlo (nucleic acid sequence)
6 ECFP (amino acid sequence)
7 ECFP (nucleic acid sequence)
8 cpVenus173 (amino acid sequence)
9 cpVenus173 (nucleic acid sequence)
10 N-terminus linker (amino acid sequence)
11 N-terminus linker (nucleic acid sequence)
12 C-terminus linker (amino acid sequence)
13 C-terminus linker (nucleic acid sequence)
14 Variant 1 truncated DhdR (amino acid sequence)
15 Variant 3 truncated DhdR (amino acid sequence)
16 Variant 1 truncated DhdR fluorescent sensor of D-2-HG (amino acid sequence)
17 Variant 3 truncated DhdR fluorescent sensor of D-2-HG (amino acid sequence)
18 Variant 2 truncated DhdR N-terminus amino acid residues
19 Variant 2 truncated DhdR C-terminus amino acid residues
20 Variant 3 truncated DhdR N-terminus amino acid residues
21 Variant 3 truncated DhdR C-terminus amino acid residues
22 Variant 1 truncated DhdR N-terminus amino acid residues
23 Variant 1 truncated DhdR C-terminus amino acid residues
24 Parent sensor forward primer
25 Parent sensor reverse primer
26 Variant 1 forward primer
27 Variant 1 reverse primer
28 Variant 2 forward primer
29 Variant 2 reverse primer
30 Variant 3 forward primer
31 Variant 3 reverse primer
32 T52A forward primer
33 T52A reverse primer
34 S69A forward primer
35 S69A reverse primer
36 S69T forward primer
37 S69T reverse primer
38 Cyto-D2HGlo forward primer
39 Cyto-D2HGlo reverse primer
40 Nuc-D2HGlo forward primer
41 Nuc-D2HGlo reverse primer
42 Mito-D2HGlo forward primer
43 Mito-D2HGlo reverse primer

Full Length DhdR (amino acid sequence)
>SEQ ID NO: 1
MLSKSLTLTEQVARQIAGDIAEGVHSVGAKLPPGRVLAEQYGVSAAVIREATERLRAQGLIQSRQGSGSV
VVSRTGAQGFQVSAGLDDREQLASVYELRMELEGGAAALAARRRNATDLAAMAEALAALEANLDHPEQGV
EHDIAFHVAIAAATHNRYYQDLLQYLNLQLRLAVSTARTNSRRQEGLTAVVHQEHVAVYDAILAGDPDRA
RLAATRHLQQAASRLRLDLLSPAARQTS
Variant 2 truncated DhdR (amino acid sequence)
>SEQ ID NO: 2
SKSLTLTEQVARQIAGDIAEGVHSVGAKLPPGRVLAEQYGVSAAVIREATERLRAQGLIQSRQGSGSVVV
SRTGAQGFQVSAGLDDREQLASVYELRMELEGGAAALAARRRNATDLAAMAEALAALEANLDHPEQGVEH
DIAFHVAIAAATHNRYYQDLLQYLNLQLRLAVSTARTNSRRQEGLTAVVHQEHVAVYDAILAGDPDRARL
AATRHLQQAASRLRLDLLSPAARQ
Variant 2 truncated DhdR (nucleic acid sequence)
>SEQ ID NO: 3
AGCAAGAGCCTGACCTTGACCGAACAGGTCGCCCGCCAGATCGCGGGCGACATCGCCGAAGGCGTCCATT
CCGTGGGCGCCAAGCTGCCGCCCGGCCGTGTCCTGGCGGAGCAGTACGGTGTGAGCGCCGCGGTCATCCG
CGAGGCCACCGAGCGCCTGCGCGCCCAGGGGCTGATCCAGAGCCGCCAGGGCTCGGGCAGCGTGGTGGTG
TCCCGCACCGGTGCTCAGGGCTTCCAGGTTTCCGCCGGCCTCGACGATCGCGAGCAGCTGGCCAGCGTCT
ACGAATTGCGGATGGAACTGGAAGGCGGCGCGGCCGCCCTGGCGGCGAGGCGCCGCAACGCCACCGACCT
TGCGGCCATGGCCGAGGCCCTGGCCGCGCTGGAAGCGAACCTGGACCATCCGGAACAGGGCGTCGAGCAC
GACATCGCCTTCCACGTCGCCATCGCCGCCGCCACGCACAACCGTTATTACCAGGACCTGCTGCAGTACC
TGAACCTGCAGCTGCGCCTGGCCGTCAGCACCGCGCGCACCAACAGCCGCCGTCAGGAGGGCCTGACCGC
GGTGGTGCACCAGGAACACGTGGCCGTCTACGACGCCATCCTCGCGGGCGATCCCGACCGCGCCCGACTG
GCGGCGACCCGCCACTTGCAGCAGGCGGCCAGCCGCCTGCGTCTCGATCTCCTCTCTCCGGCCGCAAGGC
AG
D2HGlo (amino acid sequence)
>SEQ ID NO: 4
MVSKGEELFTGVVPILVELDGDVNGHRFSVSGEGEGDATYGKLTLKFICTTGKLPVPWPTLVTTLTWGVQ
CFSRYPDHMKQHDFFKSAMPEGYVQERTIFFKDDGNYKTRAEVKFEGDTLVNRIELKGIDFKEDGNILGH
KLEYNYISHNVYITADKQKNGIKAHFKIRHNIEDGSVQLADHYQQNTPIGDGPVLLPDNHYLSTQSALSK
DPNEKRDHMVLLEFVTAARMHSKSLTLTEQVARQIAGDIAEGVHSVGAKLPPGRVLAEQYGVSAAVIREA
TERLRAQGLIQSRQGSGSVVVSRTGAQGFQVSAGLDDREQLASVYELRMELEGGAAALAARRRNATDLAA
MAEALAALEANLDHPEQGVEHDIAFHVAIAAATHNRYYQDLLQYLNLQLRLAVSTARTNSRRQEGLTAVV
HQEHVAVYDAILAGDPDRARLAATRHLQQAASRLRLDLLSPAARQELMDGGVQLADHYQQNTPIGDGPVL
LPDNHYLSYQSALSKDPNEKRDHMVLLEFVTAAGITLGMDELYKGGSGGMVSKGEELFTGVVPILVELDG
DVNGHKFSVSGEGEGDATYGKLTLKLICTTGKLPVPWPTLVTTLGYGLQCFARYPDHMKQHDFFKSAMPE
GYVQERTIFFKDDGNYKTRAEVKFEGDTLVNRIELKGIDFKEDGNILGHKLEYNYNSHNVYITADKQKNG
IKANFKIRHNIE
D2HGlo (nucleic acid sequence)
>SEQ ID NO: 5
ATGGTGAGCAAGGGCGAGGAGCTGTTCACCGGGGTGGTGCCCATCCTGGTCGAGCTGGACGGCGACGTAA
ACGGCCACAGGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTGGGGCGTGCAG
TGCTTCAGCCGCTACCCCGACCACATGAAGCAGCACGACTTCTTCAAGTCCGCCATGCCCGAAGGCTACG
TCCAGGAGCGTACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCAC
AAGCTGGAGTACAACTACATCAGCCACAACGTCTATATCACCGCCGACAAGCAGAAGAACGGCATCAAGG
CCCACTTCAAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCCTGAGCAAA
GACCCCAACGAGAAGCGCGATCACATGGTCCTGCTGGAGTTCGTGACCGCCGCCCGCATGCATAGCAAGA
GCCTGACCTTGACCGAACAGGTCGCCCGCCAGATCGCGGGCGACATCGCCGAAGGCGTCCATTCCGTGGG
CGCCAAGCTGCCGCCCGGCCGTGTCCTGGCGGAGCAGTACGGTGTGAGCGCCGCGGTCATCCGCGAGGCC
ACCGAGCGCCTGCGCGCCCAGGGGCTGATCCAGAGCCGCCAGGGCTCGGGCAGCGTGGTGGTGTCCCGCA
CCGGTGCTCAGGGCTTCCAGGTTTCCGCCGGCCTCGACGATCGCGAGCAGCTGGCCAGCGTCTACGAATT
GCGGATGGAACTGGAAGGCGGCGCGGCCGCCCTGGCGGCGAGGCGCCGCAACGCCACCGACCTTGCGGCC
ATGGCCGAGGCCCTGGCCGCGCTGGAAGCGAACCTGGACCATCCGGAACAGGGCGTCGAGCACGACATCG
CCTTCCACGTCGCCATCGCCGCCGCCACGCACAACCGTTATTACCAGGACCTGCTGCAGTACCTGAACCT
GCAGCTGCGCCTGGCCGTCAGCACCGCGCGCACCAACAGCCGCCGTCAGGAGGGCCTGACCGCGGTGGTG
CACCAGGAACACGTGGCCGTCTACGACGCCATCCTCGCGGGCGATCCCGACCGCGCCCGACTGGCGGCGA
CCCGCCACTTGCAGCAGGCGGCCAGCCGCCTGCGTCTCGATCTCCTCTCTCCGGCCGCAAGGCAGGAGCT
CATGGACGGCGGCGTGCAGCTCGCCGACCACTACCAGCAGAACACCCCCATCGGCGACGGCCCCGTGCTG
CTGCCCGACAACCACTACCTGAGCTACCAGTCCGCCCTGAGCAAAGACCCCAACGAGAAGCGCGATCACA
TGGTCCTGCTGGAGTTCGTGACCGCCGCCGGGATCACTCTCGGCATGGACGAGCTGTACAAGGGTGGCAG
CGGTGGCATGGTGAGCAAGGGCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGCCACCTACGGCAAGCTGACCC
TGAAGCTGATCTGCACCACCGGCAAGCTGCCCGTGCCCTGGCCCACCCTCGTGACCACCCTGGGCTACGG
CCTGCAGTGCTTCGCCCGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACCCGCGCCGAGGTGAAGT
TCGAGGGCGACACCCTGGTGAACCGCATCGAGCTGAAGGGCATCGACTTCAAGGAGGACGGCAACATCCT
GGGGCACAAGCTGGAGTACAACTACAACAGCCACAACGTCTATATCACCGCCGACAAGCAGAAGAACGGC
ATCAAGGCCAACTTCAAGATCCGCCACAACATCGAG
ECFP (amino acid sequence)
>SEQ ID NO: 6
MVSKGEELFTGVVPILVELDGDVNGHRFSVSGEGEGDATYGKLTLKFICTTGKLPVPWPTLVTTLTWGVQ
CFSRYPDHMKQHDFFKSAMPEGYVQERTIFFKDDGNYKTRAEVKFEGDTLVNRIELKGIDFKEDGNILGH
KLEYNYISHNVYITADKQKNGIKAHFKIRHNIEDGSVQLADHYQQNTPIGDGPVLLPDNHYLSTQSALSK
DPNEKRDHMVLLEFVTAA
ECFP (nucleic acid sequence)
>SEQ ID NO: 7
ATGGTGAGCAAGGGCGAGGAGCTGTTCACCGGGGTGGTGCCCATCCTGGTCGAGCTGGACGGCGACGTAA
ACGGCCACAGGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTGGGGCGTGCAG
TGCTTCAGCCGCTACCCCGACCACATGAAGCAGCACGACTTCTTCAAGTCCGCCATGCCCGAAGGCTACG
TCCAGGAGCGTACCATCTTCTTCAAGGACGACGGCAACTACAAGACCCGCGCCGAGGTGAAGTTCGAGGG
CGACACCCTGGTGAACCGCATCGAGCTGAAGGGCATCGACTTCAAGGAGGACGGCAACATCCTGGGGCAC
AAGCTGGAGTACAACTACATCAGCCACAACGTCTATATCACCGCCGACAAGCAGAAGAACGGCATCAAGG
CCCACTTCAAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCCTGAGCAAA
GACCCCAACGAGAAGCGCGATCACATGGTCCTGCTGGAGTTCGTGACCGCCGCC
cpVenus173 (amino acid sequence)
>SEQ ID NO: 8
MDGGVQLADHYQQNTPIGDGPVLLPDNHYLSYQSALSKDPNEKRDHMVLLEFVTAAGITLGMDELYKGGS
GGMVSKGEELFTGVVPILVELDGDVNGHKFSVSGEGEGDATYGKLTLKLICTTGKLPVPWPTLVTTLGYG
LQCFARYPDHMKQHDFFKSAMPEGYVQERTI FFKDDGNYKTRAEVKFEGDTLVNRIELKGIDFKEDGNI
LGHKLEYNYNSHNVYITADKQKNGIKANFKIRHNIE
cpVenus173 (nucleic acid sequence)
>SEQ ID NO: 9
ATGGACGGCGGCGTGCAGCTCGCCGACCACTACCAGCAGAACACCCCCATCGGCGACGGCCCCGTGCTGC
TGCCCGACAACCACTACCTGAGCTACCAGTCCGCCCTGAGCAAAGACCCCAACGAGAAGCGCGATCACAT
GGTCCTGCTGGAGTTCGTGACCGCCGCCGGGATCACTCTCGGCATGGACGAGCTGTACAAGGGTGGCAGC
GGTGGCATGGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCTGACCCT
GAAGCTGATCTGCACCACCGGCAAGCTGCCCGTGCCCTGGCCCACCCTCGTGACCACCCTGGGCTACGGC
CTGCAGTGCTTCGCCCGCTACCCCGACCACATGAAGCAGCACGACTTCTTCAAGTCCGCCATGCCCGAAG
GCTACGTCCAGGAGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGACGGCAACATCCTG
GGGCACAAGCTGGAGTACAACTACAACAGCCACAACGTCTATATCACCGCCGACAAGCAGAAGAACGGCA
TCAAGGCCAACTTCAAGATCCGCCACAACATCGAG
N-terminus linker (amino acid sequence)
>SEQ ID NO: 10
RMH
N-terminus linker (nucleic acid sequence)
>SEQ ID NO: 11
CGCATGCAT
C-terminus linker (amino acid sequence)
>SEQ ID NO: 12
EL
C-terminus linker (nucleic acid sequence)
>SEQ ID NO: 13
GAGCTC
Variant 1 truncated DhdR (amino acid sequence)
>SEQ ID NO: 14
LSKSLTLTEQVARQIAGDIAEGVHSVGAKLPPGRVLAEQYGVSAAVIREATERLRAQGLIQSRQGSGSVV
VSRTGAQGFQVSAGLDDREQLASVYELRMELEGGAAALAARRRNATDLAAMAEALAALEANLDHPEQGVE
HDIAFHVAIAAATHNRYYQDLLQYLNLQLRLAVSTARTNSRRQEGLTAVVHQEHVAVYDAILAGDPDRAR
LAATRHLQQAASRLRLDLLSPAARQT
Variant 3 truncated DhdR (amino acid sequence)
>SEQ ID NO: 15
KSLTLTEQVARQIAGDIAEGVHSVGAKLPPGRVLAEQYGVSAAVIREATERLRAQGLIQSRQGSGSVVVS
RTGAQGFQVSAGLDDREQLASVYELRMELEGGAAALAARRRNATDLAAMAEALAALEANLDHPEQGVEHD
IAFHVAIAAATHNRYYQDLLQYLNLQLRLAVSTARTNSRRQEGLTAVVHQEHVAVYDAILAGDPDRARLA
ATRHLQQAASRLRLDLLSPAAR
Variant 1 truncated DhdR fluorescent sensor of D-2-HG
>SEQ ID NO: 16
MVSKGEELFTGVVPILVELDGDVNGHRFSVSGEGEGDATYGKLTLKFICTTGKLPVPWPTLVTTLTWGVQ
CFSRYPDHMKQHDFFKSAMPEGYVQERTIFFKDDGNYKTRAEVKFEGDTLVNRIELKGIDFKEDGNILGH
KLEYNYISHNVYITADKQKNGIKAHFKIRHNIEDGSVQLADHYQQNTPIGDGPVLLPDNHYLSTQSALSK
DPNEKRDHMVLLEFVTAARMHLSKSLTLTEQVARQIAGDIAEGVHSVGAKLPPGRVLAEQYGVSAAVIRE
ATERLRAQGLIQSRQGSGSVVVSRTGAQGFQVSAGLDDREQLASVYELRMELEGGAAALAARRRNATDLA
AMAEALAALEANLDHPEQGVEHDIAFHVAIAAATHNRYYQDLLQYLNLQLRLAVSTARTNSRRQEGLTAV
VHQEHVAVYDAILAGDPDRARLAATRHLQQAASRLRLDLLSPAARQTELMDGGVQLADHYQQNTPIGDGP
VLLPDNHYLSYQSALSKDPNEKRDHMVLLEFVTAAGITLGMDELYKGGSGGMVSKGEELFTGVVPILVEL
DGDVNGHKFSVSGEGEGDATYGKLTLKLICTTGKLPVPWPTLVTTLGYGLQCFARYPDHMKQHDFFKSAM
PEGYVQERTIFFKDDGNYKTRAEVKFEGDTLVNRIELKGIDFKEDGNILGHKLEYNYNSHNVYITADKQK
NGIKANFKIRHNIE
Variant 3 truncated DhdR fluorescent sensor of D-2-HG
>SEQ ID NO: 17
MVSKGEELFTGVVPILVELDGDVNGHRFSVSGEGEGDATYGKLTLKFICTTGKLPVPWPTLVTTLTWGVQ
CFSRYPDHMKQHDFFKSAMPEGYVQERTIFFKDDGNYKTRAEVKFEGDTLVNRIELKGIDFKEDGNILGH
KLEYNYISHNVYITADKQKNGIKAHFKIRHNIEDGSVQLADHYQQNTPIGDGPVLLPDNHYLSTQSALSK
DPNEKRDHMVLLEFVTAARMHKSLTLTEQVARQIAGDIAEGVHSVGAKLPPGRVLAEQYGVSAAVIREAT
ERLRAQGLIQSRQGSGSVVVSRTGAQGFQVSAGLDDREQLASVYELRMELEGGAAALAARRRNATDLAAM
AEALAALEANLDHPEQGVEHDIAFHVAIAAATHNRYYQDLLQYLNLQLRLAVSTARTNSRRQEGLTAVVH
QEHVAVYDAILAGDPDRARLAATRHLQQAASRLRLDLLSPAARELMDGGVQLADHYQQNTPIGDGPVLLP
DNHYLSYQSALSKDPNEKRDHMVLLEFVTAAGITLGMDELYKGGSGGMVSKGEELFTGVVPILVELDGDV
NGHKFSVSGEGEGDATYGKLTLKLICTTGKLPVPWPTLVTTLGYGLQCFARYPDHMKQHDFFKSAMPEGY
VQERTIFFKDDGNYKTRAEVKFEGDTLVNRIELKGIDFKEDGNILGHKLEYNYNSHNVYITADKQKNGIK
ANFKIRHNIE
Variant 2 truncated DhdR N-terminus amino acid residues
>SEQ ID NO: 18
SKSL
Variant 2 truncated DhdR C-terminus amino acid residues
>SEQ ID NO: 19
QRAA
Variant 3 truncated DhdR N-terminus amino acid residues
>SEQ ID NO: 20
KSLT
Variant 3 truncated DhdR C-terminus amino acid residues
>SEQ ID NO: 21
RAAP
Variant 1 truncated DhdR N-terminus amino acid residues
>SEQ ID NO: 22
LSKS
Variant 1 truncated DhdR C-terminus amino acid residues
>SEQ ID NO: 23
TQRA
Parent sensor forward primer
>SEQ ID NO: 24
CATCATGCATGCATATGCTGAGCAAGAGCCTGACC
Parent sensor reverse primer
>SEQ ID NO: 25
ATGATGGAGCTCTGATGTCTGCCTTGCGGCC
Variant 1 forward primer
>SEQ ID NO: 26
CATCATGCATGCATCTGAGCAAGAGCCTGACCTTGAC
Variant 1 reverse primer
>SEQ ID NO: 27
ATGATGGAGCTCTGTCTCTGCCTTGCGGCCG
Variant 2 forward primer
>SEQ ID NO: 28
CATCATGCATGCATAGCAAGAGCCTGACCTTGACCG
Variant 2 reverse primer
>SEQ ID NO: 29
ATGATGGAGCTCCTGCCTTGCGGCCG
Variant 3 forward primer
>SEQ ID NO: 30
CATCATGCATGCATAAGAGCCTGACCTTGACCGAAC
Variant 3 reverse primer
>SEQ ID NO: 31
ATGATGGAGCTCCCTTGCGGCCGGAGAGAG
T52A forward primer
>SEQ ID NO: 32
CCGCGAGGCCGCCGAGCGCC
T52A reverse primer
>SEQ ID NO: 33
ATGACCGCGGCGCTCACACCG
S69A forward primer
>SEQ ID NO: 34
GCCAGGGCTCGGGCGCCCGTGGTGGTGTCCCGC
S69A reverse primer
>SEQ ID NO: 35
GGCTCTGGATCAGCCCCTGGGCGC
S69T forward primer
>SEQ ID NO: 36
GCCAGGGCTCGGGCACCGTGGTGGTGTCCC
S69T reverse primer
>SEQ ID NO: 37
GGCTCTGGATCAGCCCCTGGGCGC
Cyto-D2HGlo forward primer
>SEQ ID NO: 38
CATCATGGATCCCATGGTGAGCAAGGGCGAGG
Cyto-D2HGlo reverse primer
>SEQ ID NO: 39
GCCAAAACAGCCAAGCTTCGAATTC
Nuc-D2HGlo forward primer
>SEQ ID NO: 40
CATCATGGTACCATGCCTAAAAAAAAACGTAAAGTTGAAGATGCTTCGGATCCCATGGTGAGCAAGGGCG
AGG
Nuc-D2HGlo reverse primer
>SEQ ID NO: 41
GCCAAAACAGCCAAGCTTCGAATTC
Mito-D2HGlo forward primer
>SEQ ID NO: 42
CATCATGGATCCCATGGTGAGCAAGGGCGAGG
Mito-D2HGlo reverse primer
>SEQ ID NO: 43
GCCAAAACAGCCAAGCTTCGAATTC

### Inventive Results and Description Thereof

### Development of a Genetically Encoded Fluorescent Sensor for D-2-hydroxyglutarate

The transcription factor D-2-HG dehydrogenase regulator (DhdR) was recently identified as a D-2-HG-binding protein and key mediator of D-2-HG catabolismin Achromobacter denitrificans, NBRC 15125 (Xiao et al. 2021). To engineer a genetically encoded fluorescent sensor of D-2-HG, the inventive DH2Glo sensor leverages DhdR as a D-2-HG binding domain.

Inventive DH2Glo is designed as a fluorescence resonance energy transfer (FRET)-based sensor by inserting DhdR between the fluorescent proteins Enhanced Cyan Fluorescent Protein (ECFP) and a circularly permuted variant of Venus (cp173Venus; cpV173). It was predicted that DhdR would bind D-2-HG, eliciting a conformational change resulting in an altered FRET signal (FIG. 1A). To test this, the invention purifies purified the DhdR-based fluorescent sensor and exposes the sensor to increasing concentrations of D-2-HG, ranging from 10 nM to 1 mM. The FRET ratio was determined by exciting the purified protein at 440 nm and dividing the fluorescent signal collected at 531 nm by the fluorescent signal at 480 nm. It is found that at concentrations greater than 100 nM D-2-HG, the FRET ratio increased in a dose-dependent manner until plateauing around 100 μM D-2-HG (FIG. 1B). The apparent binding affinity (Kd′) of this sensor for D-2-HG was 4.00±1.64 μM, with an estimated linear range of 1 μM-50 μM. The dynamic range, defined as the maximum FRET ratio (Rₘₐₓ) divided by the minimum FRET ratio (Rₘᵢₙ), (Rmax/Rmin) of the initial FRET construct was 1.37, which is low compared with FRET-based sensors developed for other cellular metabolites (Ewald et al., 2011), (Lüddecke et al., 2017).

The binding properties of the initial FRET sensor suggested that it was suited to detect D-2-HG in the reported physiological range (Table 2). However, the dynamic range was less than other FRET-based sensors developed for similar metabolites. In an effort to improve the dynamic range, the invention manipulated the linker regions connecting DhdR with both fluorescent proteins. The inventors generated three sensor variants by systematically removing one amino acid at a time from both the N-terminus and C-terminus of DhdR and tested each variant in comparison to the original construct. The Kd′ for D-2-HG remained relatively unchanged (FIG. 1A & 1B); however, Variant 2 exhibited a significantly greater dynamic range compared with the parent sensor and the other two variants (FIG. 1B & 1C). The invention named and describes this variant as D2HGlo, which has an affinity of 3.52 μM for D-2-HG and an in vitro dynamic range of 1.66. The emission spectrum of D2HGlo, when exposed to different concentrations of D-2-HG, demonstrates that the sensor performs as predicted (FIG. 10). Purified D2HGlo was exposed to concentrations of D-2-HG, ranging from 100 nM to 500 nM, before the FRET ratio was measured (FIG. 11). At concentrations as low as 150-200 nM, there is a difference in FRET ratio compared with 100 nM. Testing of the invention finds that the lower limit of detection (LOD) for D2HGlo is 250 nM (FIG. 11A). When purified D2HGlo was exposed to concentrations of D-2-HG ranging from 50 nM to 500 nM, the LOD was determined to be 308 nM (FIG. 11B). Thus, it is estimated that the range of detection would be between 250 nM and 100 μM. The emission spectrum also revealed that the rise in FRET ratio was due to a drop in fluorescent signal at 485nm and concomitant increase in the fluorescent signal at 531 nm (FIG. 10).

### In Vitro Characterization of Purified D2HGlo

The inventors next sought to characterize the specificity of D2HGlo by evaluating its response to six Citric Acid Cycle intermediates and three additional metabolites that structurally resemble D-2-HG (pyruvate, lactate, and glutamate). D2HGlo did not respond to any of the metabolites at concentrations up to 100 μM (FIG. 2B and FIG. 12). These results indicate that D2HGlo has exceptional specificity for D-2-HG compared to other relevant biological molecules. The inventors then evaluated the effect of pH and temperature on D2HGlo in vitro. pH did not drastically alter the sensor's affinity for D-2-HG (FIG. 2C, Table 2 and 3); however, the dynamic range of

D2HGlo was decreased at pH 6.5 and increased at pH 8 relative to measurements performed at pH 7.4. Lastly, the inventors assessed if incubating the sensor with ligand at increased temperatures of 30° C. or 37° C. impacted the ability of D2HGlo to reliably quantify D-2-HG in comparison to experiments performed at room temperature. No significant difference in K_{d}′ or dynamic range was found when comparing room temperature and 30° C. or 37° C. incubations

(FIG. 2D, Table 3). Thus, D2HGlo specifically detects D-2-HG over normally occurring metabolites at physiological pH and at temperatures amenable for in vitro or in situ studies. The crystal structure for DhdR has recently been solved (Protein Data Base entry 9JPJ), which demonstrates that zinc may be a critical cofactor in binding D-2-HG. To assess the impact of zinc availability on D2HGlo function, the inventors performed D-2-HG titrations in a background of 1 mM EDTA (FIG. 18A). The inventors found that chelating zinc prevents D-2-HG induced changes to FRET ratio, regardless of the concentration (FIG. 18B). These results indicate that the presence of zinc is required for D2HGlo function.

### In Vitro Characterization of the Response of D2HGlo to L-2-HG

The production of D-2-HG in human cancers occurs due to the neomorphic enzymatic activity of mutant IDH1/2 utilizing α-ketoglutarate as a substrate. Alternatively, L-2-HG is a product of the promiscuous activity of lactate dehydrogenase (LDH) and malate dehydrogenase (MDH) (Rzem et al., 2007), (Intlekofer et al., 2017) (FIG. 3A). For D2HGlo to be considered a robust analytical tool for measuring D-2-HG in biological samples, discrimination between D-2-HG and L-2-HG is essential. Therefore, the inventors directly compared D-2-HG and L-2-HG binding in vitro (FIG. 3B). The inventors found that the K_{d}′ of D2HGlo for L-2-HG was 34.3±4.95 μM, compared with a K_{d}′ of 3.52±0.839 μM for D-2-HG. Thus, D2HGlo has a ten-fold greater affinity for D-2-HG compared with its enantiomer L-2-HG. The concentration of L-2-HG in the plasma and cerebrospinal fluid (CSF) of healthy humans typically ranges between 0.1 and 1 μM (Strain et al., 2020), (Kranendijk et al., 2012), (Gibson et al., 1993) (Bories et al., 2009). Testing of the invention found that at 1 μM, L-2-HG only elicited a 1.43% increase in the FRET ratio above baseline levels, compared with an 8.22% increase in response to the same concentration of D-2-HG (FIG. 3C). In a background of 1 μM L-2-HG, the affinity of D2HGlo for D-2-HG remained unaltered (FIG. 13). Individuals with IDH1/2 mutations have elevated levels of D-2-HG that can accumulate to more than 100 μM in fluids such as CSF (Kalinina et al., 2016). There is evidence of L-2-HG levels up to approximately 6 μM in patients with either IDH1/2 wildtype or mutant glioma (Fujita et al., 2022), (Kalinina et al., 2016). Thus, D2HGlo was exposed to 10 μM L-2-HG or D-2-HG, resulting in a 9.58% and 47.3% increase, respectively. While 10 μM L-2-HG did induce a response, concentrations of L-2-HG above this level have rarely been reported in body fluids of individuals without an oncogenic IDH mutation or metabolic disorder such as L-2-hydroxyglutaric aciduria. Therefore, D2HGlo is highly specific for D-2-HG over L-2-HG and can accurately measure D-2-HG at physiologically relevant levels.

To further assess specificity, the inventors evaluated the response of D2HGlo to six Citric Acid Cycle intermediates and three other metabolites structurally resembling D-2-HG (pyruvate, lactate, and glutamate). D2HGlo did not respond to any of the metabolites tested at a 100 μM concentration (FIG. 3D, FIG. 12). These results indicate that D2HGlo has exquisite specificity to D-2-HG compared to other relevant biomolecules. As the pH of biological fluids may vary, the inventors evaluated the effect of pH on D2HGlo in vitro (FIG. 3E). As shown in Table 2 below, pH did not drastically alter the sensor's affinity for D-2-HG; however, the dynamic range was decreased at pH 6.5 and increased at pH 8 relative to measurements performed at pH 7.4. These results show that D2HGlo has remarkable sensitivity and specificity for D-2-HG at physiological pH and physiologically relevant concentrations of D-2-HG.

TABLE 2
In vitro D2HGlo characterization at different pH values.
Values shown are the average of three independent experiments.
The Kd′ of D2HGlo remained unchanged across three
different pH values. In contrast, the dynamic range was
dramatically decreased at pH 6.5 and was increased at
pH 8, relative to measurements performed at pH 7.4.

| | K_{d} (μM) | Dynamic Range |
|---|---|---|
| pH 6.5 | 3.51 ± 0.398 | 1.35 ± 0.0205 |
| pH 7.4 | 3.52 ± 0.839 | 1.66 ± 0.0248 |
| pH 8 | 3.25 ± 0.424 | 1.80 ± 0.0245 |

TABLE 3
In vitro characterization of D2HGlo at different pH and temperatures.
The table shows the results of table 1 to 2 d.p, plus the K_{d}′
and dynamic range of D2HGlo at 30° C. or 37° C. and pH 7.4.

| | K_{d}′ (μM) | Dynamic Range |
|---|---|---|
| pH 6.5 | 3.51 ± 0.40 | 1.35 ± 0.02 |
| pH 7.4 | 3.36 ± 0.67 | 1.67 ± 0.03 |
| pH 8 | 3.25 ± 0.42 | 1.80 ± 0.02 |
| 30° C. (pH 7.4) | 2.77 ± 0.12 | 1.67 ± 0.06 |
| 37° C. (pH 7.4) | 3.26 ± 0.53 | 1.69 ± 0.07 |

To further assess the impact of a physiologically relevant concentration of L-2-HG on the ability of D2HGlo to quantify D-2-HG, D-2-HG titrations were performed in a background of L-2-HG ranging from 1.5-5.5 μM (FIG. 4D). As expected, the inventors did not observe a significant change in the dynamic range or the K_{d}′ (FIG. 4E and FIG. 4F). In a background of higher concentrations of L-2-HG ranging from 15-55 μM, the dynamic range gradually decreased until plateauing at ˜1.3 at background L-2-HG concentrations ranging from 35-55 μM (FIG. 4E). Interestingly, higher concentrations of L-2-HG (15-55 μM) modestly increased the K_{d}′ for D-2-HG by approximately 2 M, demonstrating that an excess background and competition from L-2-HG only slightly inhibited D-2-HG binding (FIG. 4F).

### D2HGlo Detects D-2-HG in Living Cells

Current methods for assessing D-2-HG levels are limited to fluids or supernatants; as such, D-2-HG has never been detected in living cells. To investigate if D2HGlo can be used to detect cellular D-2-HG, the inventors developed a cytosolic version of D2HGlo for expression in mammalian cells. Cyto-D2HGlo displayed proper localization when expressed in HeLa cells, as evidenced by its nuclear exclusion (FIG. 5A). Permeabilized HeLa cells expressing Cyto-D2HGlo were treated with 1 mM D-2-HG. The inventors observed a dramatic increase in the FRET ratio to a level 1.37-fold above baseline (FIG. 5B, 3C, 3E). Importantly, this demonstrates the successful use of D2HGlo in situ. In contrast, 1 mM L-2-HG only elicited a modest 1.23-fold rise (FIG. 5B, 3D, 3E). Additionally, the inventors noted a substantial delay between 2-HG addition and the observed rise in FRET ratio for both 2-HG enantiomers; however, this interval appeared to be far greater for L-2-HG. To assess whether the concentration of exogenous D-2-HG affected the magnitude of the in situ response of Cyto-D2HGlo, the inventors repeated our cellular measurements with 10 mM, 0.1 mM, and 0.01 mM D-2-HG (FIG. 5C). The inventors found that D-2-HG at 0.1 mM and 10 mM resulted in a comparable response to 1 mM, suggesting Cyto-D2HGlo was saturated at these concentrations. In contrast, 0.01 mM D-2-HG elicited a moderate FRET response above baseline (1.09-fold). Thus, Cyto-D2HGlo responds to exogenously applied D-2-HG in a dose-dependent manner (FIG. 5E). Importantly, Cyto-D2HGlo did not respond to L-2-HG at concentrations less than 1 mM (FIG. 5D). This evidence further supports our in vitro finding that D2HGlo is specific for physiologically relevant concentrations of D-2-HG relative to L-2-HG.

### Illumination of Nuclear and Mitochondrial D-2-HG Using D2HGlo

IDH1-R132H is expressed in the cytosol of cells, yet many of the deleterious oncogenic effects of D-2-HG are postulated to occur in the nucleus (Kusi et al., 2022), (Chowdhury et al., 2011), (Yang et al., 2017), (Ceccarelli et al., 2016), (Turcan et al., 2018). IDH2 is present in the mitochondrial matrix and is a key player in the Krebs cycle. There is evidence that D-2-HG interferes with transcription in the nucleus. Additionally, D-2-HG is degraded in the mitochondria by D-2-HG dehydrogenase (Ye et al., 2018). However, the presence of D-2-HG in the nucleus or mitochondria has not been demonstrated. To test the feasibility of using D2HGlo to interrogate cellular D-2-HG, the inventors developed a cytosolic cellular probe, Cyto-D2HGlo, and assessed its response to exogenously applied D-2-HG and L-2-HG in HeLa cells. Cyto-D2HGlo responded to concentrations of D-2-HG ranging from 10 μM-10 mM, however, a significant difference in discrimination between D-2-HG and L-2-HG only occurred at a concentration of 100 μM (FIG. 5). The inventors developed two additional cellular D2HGlo probes that were targeted to the nucleus (Nuc-D2HGlo) and the mitochondria (Mito-D2HGlo). The inventors validated that the nuclear and mitochondrial sensors exhibited proper localization in HeLa and IDH1 mutant U87MG cells (FIG. 19) and wild type IDH1 U87MG cells (FIG. 6). Cyto-D2HGlo, Nuc-D2HGlo, and Mito-D2HGlo were expressed and evaluated in IDH1 wildtype and IDH1-R132H mutant U87MG cells. By calculating the FRET ratio for each condition, the inventors compared the relative endogenous D-2-HG concentrations in IDH1 wildtype and mutant cells (FIG. 6A-C). It was found that the FRET ratio was significantly elevated in the mutant cells compared with the wildtype in all three cellular compartments, suggesting that D-2-HG levels are elevated in the cytosol, nucleus, and mitochondria of IDH1 mutant cells. Since the FRET ratio is a direct readout of D-2-HG concentration, these results suggest that IDH1 mutations drive accumulation of D-2-HG in compartments other than the cytosol, including the nucleus where D-2-HG is known to have considerable epigenetic implications. Notably, this is the first time mutant IDH1-driven accumulation of D-2-HG has been demonstrated in the nucleus and mitochondria.

To validate the role of mutant IDH1 in the production of cellular D-2-HG, the inventors utilized AG-120, an FDA-approved mutant IDH1 inhibitor. Small molecule inhibitors of mutant IDH1 are a promising new targeted therapy effective in treating gliomas (Popovici et al., 2018) however, their effect on D-2-HG levels in living cells has never been directly examined. Real-time imaging of IDH1 mutant cells expressing Cyto-D2HGlo revealed that AG-120 did not result in acute changes to the FRET ratio over a short time period (within 15 minutes following drug addition) (FIG. 15). Therefore, the inventors elected to treat IDH1 wildtype and IDH1-R132H mutant U87MG cells with AG-120 for 48 hours prior to collecting FRET ratio images. The inventors demonstrated that AG-120 (3 μM) reduced relative FRET ratios in all three compartments of IDH1 mutant cells to levels comparable with IDH1 wildtype cells (FIG. 6A). As expected, AG-120 had no effect on D-2-HG levels in wildtype IDH1 U87MG cells. This evidence further supports the specificity of D2HGlo to endogenous D-2-HG in situ. Theoretically, the AG-120 mediated reduction of D-2-HG in the nucleus should mitigate the epigenetic reprogramming associated with IDH1 mutations. This suggests a mechanism for the efficacy of mutant IDH1 inhibitor therapy. These results are consistent with the findings of AG-120 clinical trials involving both newly diagnosed and advanced cancer, which demonstrate a reduction of 2-HG to levels similar to those seen in healthy populations (Fan et al., 2020), (Fan et al., 2020), (Fan et al., 2022).

While AG-120 was able to reduce the production of D-2-HG over a 48-hour period, no acute changes to the FRET ratio were observed. To determine whether real-time differences in cytosolic D-2-HG could be detected using Cyto-D2HGlo, the inventors targeted other potential pathways upstream of mutant IDH-driven D-2-HG production. The inventors utilized the glutaminase C inhibitor Compound 968 and 2-Deoxy-D-glucose (2-DG), an inhibitor of glycolysis. Acute application of 2-DG (10 mM) selectivity reduced the FRET ratio in IDH1-R132H mutant cells, whereas IDH1 wildtype cells were unaffected (FIG. 7a). In agreement with this finding, the concentration of D-2-HG in the supernatant of IDH1-R132H mutant cells that had been treated with 2-DG for 48 hours was reduced to levels comparable to untreated wildtype cells (FIG. 7b). Acute application of Compound 968 (1 μM) did not elicit any changes in FRET ratio in wildtype or mutant cells expressing Cyto-D2HGlo (FIG. 7c) nor did it affect D-2-HG accumulation in cell culture supernatants (FIG. 7d). Thus, D2HGlo can illuminate real-time changes in D-2-HG concentration in situ in parallel with in vitro measurements of D-2-HG levels in cellular supernatants. In this case, the evidence shows that mutant IDH-driven D-2-HG accumulation is downstream of glycolysis as opposed to glutamine catabolism

### D2HGlo Detects D-2-HG in Cell Culture Supernatant And Human Biological Fluids

Since D-2-HG is released extracellularly (Bunse et al., 2018), the inventors examined whether D2HGlo can detect differences in D-2-HG concentrations in cellular supernatants. The D-2-HG concentration of supernatant from wildtype cells had a concentration of ˜6.56 μM (FIG. 8A), while supernatant from IDH1-R132H cells contained ˜63.0 μM. As previously demonstrated in situ (FIG. 6), 48-hour pre-treatment with AG-120 reduced the D-2-HG concentration to baseline levels. A shown in FIG. 8A, Treatment of IDH1 mutant cells with AG-120 (3 μM) for 48 hours reduced the D-2-HG concentration to 2.79±0.41 μM in IDH1 wildtype cells while IDH1-R132H cells contained 2.88±0.59 μM. These results demonstrate the ability of the sensor to recognize changes in endogenous D-2-HG concentrations in biological fluids. This suggests that D2HGlo may be used to monitor the efficacy of mutant IDH1 inhibitors.

Due to the previously established ability of D2HGlo to produce reliable FRET measurements in cell culture media, as well as the clinical significance of quantifying D2-HG within biological fluids, the inventors next sought to assess sensor performance in serum, artificial cerebrospinal fluid (aCSF), and urine. The inventors spiked D-2-HG in urine, serum and aCSF at concentrations ranging from 100 nM to 1 mM and measured changes in FRET ratios (FIG. 8B-G). Importantly, no significant differences in the dynamic range were observed when comparing D2HGlo's response between buffer, serum, urine, and aCSF. Furthermore, the Kd′ of these samples showed little variation regardless of the biological matrix. This demonstrates that the performance of D2HGlo is not reduced by serum, urine, or aCSF (FIG. 8B-8G), supporting the potential use of D2HGlo to measure D-2-HG in clinical samples to detect IDH mutations and/or monitor patient therapy.

### D2HGlo Successfully Predicts Mutant IDH1 Status in the Supernatant of Patient Derived Glioma Tumor Samples

Following the successful performance of D2HGlo within various biological mediums, the inventors sought to assess the feasibility of measuring D-2-HG in the supernatant of patient-derived tumor samples. Twenty archived glioma tumor samples with IDH1 mutational status previously determined by immunohistochemistry (IHC) and colorimetric peptide nucleic acid loop-mediated isothermal amplification (CPNA-LAMP) were assessed for the presence of D-2-HG. Significant differences were observed between all IDH1 mutant and wildtype samples (FIG. 9A), with an average FRET ratio of 2.0 recorded for IDH1 mutant samples and an average ratio of 1.5 for IDH1 wildtype (FIG. 9B). Thus, the inventors successfully differentiated between IDH1 wildtype and IDH1 mutant samples using D2HGlo with 100% concordance with the pathology report (FIG. 9C). D-2-HG concentrations in IDH1-R132H mutant tumor supernatants ranged between 11.1 and 72.3 μM and were not detectable in IDH1-R132 wildtype supernatants. These results suggest that a FRET ratio of ≥1.7 (FIG. 9D), which correlates with a D-2-HG concentration of 1.87 μM, is predictive of the presence of an IDH1 mutation, supporting the feasibility of optimizing D2HGlo to intraoperatively diagnose the presence of an IDH1/2 point mutation based on elevated D-2-HG levels.

### Discussion

Quantification of D-2-HG in samples has historically been challenging, and current assays are difficult, expensive, time-consuming, and/or do not function in a broad and biologically relevant range of concentrations. Discrimination between D-2-HG and L-2-HG is often ignored (Wang et al., 2013) or difficult using commonly available techniques.

Quantification of D-2-HG can be performed using LC-MS (Fujita et al., 2022), (Tuna et al., 2022), (Struys et al., 2004), (Zhang et al., 2023), ion-paired reverse-phase liquid chromatography (Fathi et al., 2016), GC-MS (Fernández-Galán et al., 2018), and MRS (Choi et al., 2012). These methods do not easily discern between D and L-2-HG, and performance time limits their utility. Commercially available fluorimetric assays for detecting D-2-HG also exist, but require a deproteinization step and 30-60 minute incubation. A FRET sensor capable of the specific detection of D-2-HG has recently been developed (Xiao et al., 2023) but is limited by a poor dynamic range and use at a physiologically impossible pH of 10. In comparison, D2HGlo has superior performance, as demonstrated by a substantially improved dynamic range and use at the physiologically relevant pH of 7.4. Additionally, D-2-HGlo has broad utility to assess D-2-HG in various sample types.

Additionally, currently utilized techniques for the direct detection of IDH mutations such as genetic sequencing and IHC have higher cost and time requirements and are not amenable to remote testing. IHC is also limited to the detection of IDH1-R132H, whereas detection of elevated D-2-HG is inclusive of less common variants (Nakagawa et al., 2022, Pusch et al., 2014). There is an urgent need for a rapid and convenient liquid biopsy tool amenable for D-2-HG quantification in the clinical setting. Genetically encoded fluorescent sensors have been developed to quantify the concentration of metal ions and cellular metabolites in situ (Pratt et al., 2019, Pratt et al., 2021), however, they are not routinely used as an in vitro diagnostic device. A FRET sensor capable of the specific detection of D-2-HG has recently been developed (Xiao et al., 2023) but is limited by a poor dynamic range and use at a physiologically impossible pH of 10.

The inventors have developed a FRET-based sensor of D-2-HG that can reliably measure D-2-HG in living cells, biological fluids, and tumor supernatant. In comparison to the biosensor of the prior art (Xiao et al. 2023), D2HGlo performs at a physiologically relevant pH of 7.4 and quantifies D-2-HG within the currently documented disease range. Notably, D2HGlo does not respond to metabolites that are similar in structure to D-2-HG, and demonstrates clear discrimination between D-2-HG and L-2-HG at physiologically possible concentrations. The sensitivity and specificity of D-2-HGlo are remarkable, thus making it a powerful tool for investigating D-2-HG in situ and in vitro. Using D2Hglo, the inventors detected D-2-HG in subcellular compartments, including the nucleus, which has not previously been demonstrated. Furthermore, D2HGlo can rapidly quantify D-2-HG levels within a broad range of mediums, including cell culture supernatant, serum, aCSF, and supernatant derived from tumor lysates with no significant decrease in binding affinity or dynamic range in comparison to buffer. However, our results indicate that D2HGlo is not compatible with heparinized plasma or whole blood. Although the inventors were not able to acquire patient serum, urine, or CSF samples, the inventors demonstrate the feasibility of accurate quantification of D-2-HG using contrived samples in these matrices.

This assay could offer many advantages over current practices, including cost effectiveness and reducing patient distress. Results are available in approximately 15 minutes, and D-2-HG in biological fluids remains stable through several freeze-thaw cycles (Kalinina et al., 2016) or exposure to excess heat (Voelxen et al., 2016), making D2HGlo an attractive candidate for facilitating the remote diagnosis and disease monitoring of patients through mail in samples. Additionally, the inventors found D2HGlo's performance to be highly reproducible. This is demonstrated by data in FIGS. 1b-c, 2b-d, 4b-f, 8a-g, and 9a-b, which are all representative of at least three independent sensor purifications. While D2HGlo has a greater affinity for D-2-HG over L-2-HG, it is recognized that its response to L-2-HG at higher concentrations may be a shortcoming in various potential applications. The inventors found that at L-2-HG background concentrations below 10 μM, accurate quantification of D-2-HG reliably occurs; however, it was found that accurate quantification of D-2-HG is increasingly hindered by elevated concentrations of L-2-HG (>10 μM) in a dose dependent manner. To address this, the inventors attempted to improve enantiomer selectivity with site-directed mutagenesis of predicted binding domains but were unable to lower the affinity for D-2-HG or L-2-HG. While these results suggest the inventors did not target the correct residues, it is highly probable that D-2-HG and L-2-HG share the same binding domain; thus, lowering the sensors affinity for L-2-HG will likely lower it for D-2-HG in tandem. Though D2HGlo's utility in detecting IDH1 mutations using serum, urine, and/or CSF must be assessed with clinical samples, successful discrimination between IDH1 wildtype and IDH1 mutant tumor samples was achieved in this study. L-2-HG did not appear to be present in appreciable amounts for any IDH1 wildtype sample, as the FRET ratios obtained were not indicative of detectable amounts of ligand. Further, literature values of L-2-HG in the serum and CSF of humans without IDH mutations or metabolic disorders such as L-2-hydroxyglutaric aciduria are reported as ≤6.1 μM, suggesting D2HGlo is highly compatible with quantifying D-2-HG in these matrices.

In addition to the potential clinical applications of D2HGlo, the inventors also demonstrate preliminary findings of its utility in-situ. The inventors investigated the ability of D2HGlo to recognize subcellular differences in endogenous D-2-HG between IDH1 mutant and IDH1 wildtype cells by targeting the sensor to the nucleus, mitochondria, and cytoplasm. FRET ratios were significantly elevated in IDH1 mutant cells in all three compartments, suggesting that D-2-HG accumulates throughout these cells. Our prediction that endogenous D-2-HG caused this increase in FRET ratio was supported by the inhibition of D-2-HG production through AG-120, a mutant IDH pharmaceutical inhibitor. 48-hour treatment with AG-120 caused the average FRET ratio for the nucleus, mitochondria, and cytoplasm of IDH1 mutant cells to decrease to levels resembling those found in IDH1 wildtype cells. Since no published tool currently exists to assess D-2-HG in living cells, these results offer findings of elevated D-2-HG within the nucleus, providing a potential mechanism for the abundance of epigenetic changes that are characteristic of IDH mutations. These findings also indicate that D2HGlo may be a valuable tool for further investigating the cellular distribution of D-2-HG and other cell-based studies of IDH mutant cells that have previously not been possible. However, as the crystal structure for DhdR has not been solved, the inventors were unable to generate a dead sensor as a negative control in these experiments. Further characterization, such as the impact of pH and hypoxia on performance in-situ are also warranted. The crystal structure of DhdR has recently been solved by Sun and Wang (Protein Data Base entry 9JPJ) with the related study awaiting publication. With this crystal structure, specific residues associated with D-2-HG binding have been elucidated. Thus, future characterization of in-situ D2HGlo probes will include site directed mutagenesis studies targeting residues associated with ligand binding, potentially generating sensors with altered affinity for D-2-HG and/or L-2-HG. These experiments may also prove beneficial for the specificity of D2HGlo in vitro. Additional future characterization of in-situ probes includes experiments to determine the impact of pH and hypoxia on sensor performance. Further in-situ characterization of D2HGlo probes using primary cell lines or other IDH mutant cell lines such as BT142 will also be useful in assessing variation in D-2-HG production and localization.

The presence of IDH1 mutations in glioma is associated with improved survival following aggressive surgical resection (Beiko et al., Cahill et al., 2021, Jakola et al., 2022), but no standardized intraoperative methods are currently used to identify IDH mutations. MRS may be employed to gauge total 2-HG levels preoperatively, but this technique is limited by high cost, operator dependence, and low signal-to-noise ratio which require lesions to be at least several millimeters in volume and sufficiently distant from fluid-brain or air-fluid interfaces. Various genotyping approaches have also been developed to determine the IDH mutational status within the intraoperative window (Choate et al., 2024, Shankar et al., 2018, Xue et al., 2023). As D2HGlo could accurately predict IDH1 mutational status in glioma tumor supernatants, the intraoperative use of D2HGlo may prove useful if altering the extent of resection based on IDH mutational status is desired. Importantly, the tissue requirement for this assay is modest and requires minimal processing. Using D2HGlo, the inventors successfully determined the IDH mutational status using the supernatant derived from 100 mg of each respective tumor (FIG. 9). Tissue samples were briefly homogenized in 500 μL of physiological saline prior to assaying, with a final requirement of only 30 μL per triplicate reaction. Given the reaction volume used, the inventors estimate that as a little as 10 mg of tumor tissue homogenized in 50 μL is sufficient for D2HGlo analysis.

Collectively, our results suggest that D2HGlo may be a viable tool for inferring the presence of IDH mutations preoperatively and/or to monitor the efficacy of mutant IDH inhibitor therapy through liquid biopsies. Since the inventors were unable to acquire patient serum or urine, the utility of D2HGlo for these purposes remains speculative and will require extensive clinical validation with patient samples. This validation would likely be performed in parallel with established methods for 2-HG quantification, such as MS. While quantification of 2-HG in tumor lysates with an orthogonal method such as LC-MS/GC-MS would have been useful in this study, the inventors were limited by tissue availability.

For future clinical implementation, the inventors intend to validate D2HGlo in accordance with CLIA standards for its use as a diagnostic and predictive tool and envision the development of a lyophilized sensor that can be resuspended using sample buffer, water, or patient samples. In addition to clinical applications, the inventors also present D2HGlo cellular probes which facilitated the study of D-2-HG distribution in-situ. These probes may play a valuable role in further characterizing the mechanism of D-2-HG driven oncogenesis in IDH mutant cells (Beiko et al., 2014, Cahill et al., 2021, Jakola et al., 2022, Molinaro et al., 2020, Pomorski et al., 2013).

The inventors propose that D-2-HGlo can be used to interrogate the regulation and dynamics of D-2-HG in living cells, providing a better understanding of the deleterious effects of this oncometabolite. Importantly, D-2-HGlo specifically detects D-2-HG in a biologically relevant range and is amenable to assessing the presence of IDH1/2 mutations and monitoring the efficacy of mutant IDH1 inhibitor therapy. The presence of IDH1 mutations in glioma is associated with improved survival following aggressive surgical resection (Beiko et al., 2014), (Cahill, 2021), (Jakola et al., 2022), (Molinaro et al., 2020), but no preoperative or intraoperative methods of detection are currently available. As D-2-HGlo could accurately predict IDH1 mutational status in glioma tumor supernatants, the inventors envision the intraoperative use of D-2-HGlo to detect the presence of an IDH1/2 mutation. Additionally, D-2-HGlo may prove useful in the preoperative assessment of elevated D-2-HG levels in cancer patients.

### Conclusions

The inventors have developed a first-generation FRET-based sensor that can reliably measure D-2-HG in contrived clinical specimens and tumor supernatants. Notably, D2HGlo does not respond to metabolites that are similar in structure to D-2-HG and can discriminate between D-2-HG and L-2-HG in the presence of physiologically relevant concentrations of L-2-HG. The sensitivity and specificity of D2HGlo in contrived and patient tumor samples demonstrates that it may be a powerful tool for quantifying D-2-HG via a liquid biopsy approach. Additionally, using targeted cellular probes of D2HGlo the inventors detected elevated D-2-HG in several distinct subcellular compartments of IDH1 mutant cells, including the nucleus.

### Materials & Methods

Chemicals and reagents: Oxaloacetic acid, α-ketoglutaric acid, succinic acid, L-glutamic acid, sodium hydrogen DL-malate, sodium L-lactate, and 2-Deoxy-D-glucose were purchased from Thermo Fisher Scientific (Waltham, MA). Sodium citrate was purchased from Ward's Science. Fumarate was purchased from TCI Chemical Company. Sodium pyruvate was purchased from Beantown Chemical. D-2-HG, L-2-HG, isocitrate, AG-120, 2R-Octyl-α-hydroxyglutarate, 2S-Octyl-α-hydroxyglutarate, and glutaminase inhibitor (compound 968) were purchased from Cayman Chemical (Ann Arbor, MI).

Molecular Cloning: The gene encoding the transcription factor DhdR from Achromobacter denitrificans NBRC 15125 (ADE01S_RS31870) was purchased as a gene fragment from Azenta Life Sciences (Burlington, MA). The DhdR gene was PCR amplified using primers containing the Sphl and Sacl restriction sites and inserted into a pBAD vector containing the fluorescent proteins ECFP and cpVenus173 (Supplemental Table 1). The fluorescent proteins originated from pcDNA3.1 (+)-NES-ZapCV2 (cpV143) (Addgene Plasmid #36231). The complete DNA sequence (SEQ ID NO: . . . ) and amino acid sequence (SEQ ID NO: . . . ) for D2HGlo are disclosed in the application. Three variants of the parent DhdR-based FRET sensor were generated by removing the full-length DhdR domain using Sphl and Sacl. The three truncated versions of DhdR were generated by PCR amplification and inserted in place of the parent DhdR domain. For mammalian expression of D2HGlo sensors, D2HGlo was PCR amplified from the original pBAD vector and transferred to one of two pcDNA3 backbones. To generate Cyto-D2HGlo and Nuc-D2HGlo, pcDNA3.1(+)-NES-ZapCV2 (cpV143) (Addgene Plasmid #36231) was used as a destination vector. Both D2HGlo and the vector were digested with either BamHI/EcoRI for Cyto-D2HGlo or KpnI/EcoRI for Nuc-DH2Glo. This resulted in a cytosolic D2HGlo construct with the nuclear export signal (NES) MLQLPPLERLTL at the N-terminus or a nuclear construct with the nuclear localization signal (NLS) MPKKKRKVEDA at the N-terminus. To generate Mito-D2HGlo pcDNA-mito-ZapCY (Addgene Plasmid #36231) was the destination vector and BamHI/EcoRI were used for restriction cloning. This resulted in a mitochondrial D2HGlo construct with four copies of CoxVII (YVRPDAAAAAGLDRLGPAAPSAARQDPFVG) at the N-terminus. All DNA constructs were transformed into Invitrogen™ Subcloning Efficiency™ DH5α Competent Cells, and colonies were selected and cultured in LB broth containing 100 ug/mL ampicillin. Plasmid DNA was purified using E.Z.N.A Plasmid DNA Mini Kit I (Omega Bio-Tek, D6948). All DNA constructs were confirmed by Sanger sequencing.

Site-Directed Mutagenesis: AlphaFold was utilized to convert the primary sequence of DhdR into a 3D structure, then SwissDock was used to identify the most stable binding mode for DhdR with D-2-HG or L-2-HG ligands. SwissDock identified that L-2-HG formed an interaction with T52 and D-2-HG formed an interaction with S69. PCR was then utilized to generate three constructs containing mutations anticipated to impact 2-HG binding (T52A, S69A, and S69T). Mutations were produced by incorporating the desired base pair changes into the primer sequence, amplifying the D2HGlo plasmid, and digesting the original construct with DPNI. Prepared constructs were transformed into competent bacteria followed by protein purification and sensor assessment via titrations of D-2-HG and L-2-HG as previously described. Primer sequences can be found in Supplemental Table 1.

Protein Expression and Purification: DhdR-based FRET construct (encoded by the pBAD vector) were transformed into One Shot TOP10 Chemically Competent E. coli (Thermo Fisher Scientific, Waltham, MA). Colonies were selected and grown up at 37°° C. in 2xYT microbial growth media containing 100 μg/mL ampicilin. Once the culture reached an OD₆₀₀ of approximately 0.6, protein expression was induced by the addition of 0.2% L-arabinose. Cultures were then grown at 22° C. overnight. Bacterial cell lysis was performed using B-PER reagent (Thermo Fisher Scientific) supplemented with lysozyme (50 mg/mL) and DNase | (2,500 U/mL). The resulting crude lysate was cleared by centrifugation, and the supernatant was sterile filtered. D2HGlo was purified using Cobalt NTA Affinity Resin (vendor) (Takara Bio). The resin was washed with equilibration buffer (50 mM Tris, 300 mM NaCl, pH 7.4) prior to addition of the cleared lysate. The column was rinsed with wash buffer (50 mM Tris, 300 mM NaCl, 20 mM imidazole) two times before addition of the elution buffer (50 mM Tris, 300 mM NaCl, 250 mM Imidazole). The eluent was exchanged into an experimental buffer containing 150 mM HEPES, 100 mM NaCL and 10% glycerol (pH 7.4) using PD-10 desalting columns. For pH studies, the experimental buffer was buffered at pH 6.5 or pH 8. In all experiments the concentration of purified FRET sensor was determined based on absorbance of the acceptor fluorescent protein cpVenus173 (ε=92,000 M⁻¹ cm⁻¹ at 515 nm).

In vitro characterization of purified D2HGlo: Purified D2HGlo was diluted to 5 μM in experimental buffer for all in vitro fluorescence measurements. D-2-HG was prepared as a 50 mM stock in phosphate buffered saline (PBS) pH 7.4, and serial dilutions were performed to achieve concentrations of metabolites between 10 nM and 1 mM. L-2-HG and other cellular metabolites were prepared in a similar manner. 90 μL of purified protein was mixed with 10 μL of a 10× concentration of D-2-HG in a blackwalled 96-well plate. Fluorescence measurements were performed using a Synergy Multi-Mode Plate Reader (BioTek Instruments, Winooski, VT). For the collection of emission spectra, the imaging parameters were: 440 nm excitation and 450-650 nm emission window. In some experiments, the imaging parameters for collection of emission spectra were: 420/10 nm excitation and 450-650 nm emission window (1 nm stepsize). For the D-2-HG binding studies, the imaging parameters included: 440 nm excitation/482 nm emission, 440 nm excitation/485 nm emission, and 440 nm excitation/531 emission. The FRET ratio was calculated by dividing the fluorescent signal collected at 531 nm by the fluorescent signal collected at 482 nm or 485 nm. The FRET ratio was plotted against the log₁₀ concentration of D-2-HG. The data points were fit with the following equation: FRET_{bound}*xⁿ+FRET_{unbound}*K_{d}ⁿ)/(CFP_{bound}*xⁿ+CFP_{unbound}*K_{d}ⁿ to determine the apparent binding affinity (K′_{d}), where FRET_{bound} is the maximum FRET ratio and FRET_{unbound} is the minimum FRET ratio (Pomorski et al., 2013), and where x=D-2-HG. For temperature-based characterization, varying concentrations of D-2-HG were combined with 5 μM sensor, incubated at 30° C. or 37° C. for 15 minutes, then fluorescence measurements were obtained immediately. L-2-HG and other metabolites were prepared in the same manner as D-2-HG, and binding studies were conducted in identical fashion.

In one set of experiments, D-2-HG binding was evaluated in the presence of 1 μM L-2-HG. For pH experiments, sensor was prepared in titration buffer with a final pH of 6.5, 7.5, or 8.5 then analyzed for D-2-HG binding capacity as previously specified. For EDTA experiments, D2HGlo titrations with D-2-HG were performed in a background of 1 mM EDTA prior to fluorescence measurements.

Cell Culture and Transfection. IDH1 wild-type U87MG, CRISPR edited IDH1-R132H Isogenic U87HTB-141G™ (ATCC, Manassas, VA, USA), and HeLa cells (ATCC, Manassas, Virginia) were cultured in DMEM (Lonza, Portsmouth, NH, USA) supplemented with 10% fetal bovine serum (Atlanta Biologicals, Norcross, GA, USA) and 1% penicillin/streptomycin/amphotericin B (PSA) (Lonza, Portsmouth, NH, USA) at 37° C. and 5% CO2 For imaging experiments, cells were plated in 6-well dishes at 70-80% confluence. Cells were transfected 24 hours after plating with 1.0 μg D2HGlo constructs and 3.0 μL Trans-IT®-LT1 transfection reagent (Mirus Bio, Madison, WI). The cells were allowed to express for 48 hours before imaging.

Fluorescence Live-Cell Imaging. IDH1 wild-type U87MG, CRISPR edited IDH1-R132H Isogenic U87HTB-141G™ cells and HeLa cells were imaged in 8-chamber coverslips (Ibidii, Fitchburg, WI. The media was exchanged for HEPES-buffered Hank's Balanced Salt Solution (140 mM NaCl, 5 mM KCl, 1 mM CaCl₂, 4 mM MgSO₄, 5 mM MgCl₂, 3 mM Na₂PO₄, 4 mM PO₄ 6 mM glucose, 4 mM NaHCO₃, pH 7.4). Cells expressing the D2HGlo sensors were imaged using an Olympus IX-81 microscope and FLUOVIEW Confocal Microscope. The standard imaging parameters were: 457 nm laser line and 482 nm and 531 nm emission. In some experiments cells expressing the D2HGlo sensors were imaged using an Olympus IX-81 microscope and FLUOVIEW FV3000 Confocal Microscope. The standard imaging parametersD2HGlo sensors were: 457 excited at 458 nm laser line and 482 nm and 531 nm and the fluorescent signal was collected using a BA 480-495 emission filter (ECFP) and a BA 535-565 emission filter (FRET). The FRET ratio was determined by dividing the FRET signal by the ECFP signal. For real-time FRET experiments of HeLa cells expressing Cyto-D2HGlo, a baseline FRET ratio was collected for one minute prior to addition of D-2-HG or L-2-HG at 0.01 mM, 0.1 mM, 1 mM or 10 mM. The FRET ratio was collected until a maximum FRET ratio had been achieved or the cells had died. The maximum FRET ratio was determined for the entire time-course and normalized to the baseline FRET ratio. For long-term FRET experiments, ratiometric FRET images of U87MG or CRISPR edited IDH1-R132H Isogenic U87HTB-141G™ cells that had been transfected with Cyto-D2HGlo, Nuc-D2HGlo or Mito-D2HGlo were collected. A region of interest was drawn on each cell, and the average FRET ratio within each ROI was determined. In some sets of experiments, cells were treated with AG-120 (3 μM) for 48 hours prior to FRET ratio collection. Alternatively, glutaminase inhibitor (1 μM), or 2-DG (10 mM) were added to imaging wells after collecting baseline FRET values for 30 seconds.

Cell Viability Assays: U87MG, IDH1-R132H mutant U87MG, and SVG (ATCC, Manassas, VA, CRL-8621) cells were plated at 10,000 cells per well in an opaque 96-well plate with complete media and incubation conditions as described previously. Cells were incubated overnight, then media was then replaced with DMEM containing 10% FBS, 1% PSA, and a concentration of cell permeable D-2-HG (2R-Octyl-α-hydroxyglutarate), or L-2-HG (2S-Octyl-α hydroxyglutarate) ranging from 4 mM-0 μM. Cells were incubated for 48 hours, then assessed for viability with CellTiter-Glo® (Promega) according to protocol. Luminescence was measured on a Synergy Multi-Mode Plate Reader.

Cell Culture Supernatant: U87MG IDH1 wildtype and mutant cells were plated at 60% confluence in 6-well plates. For some experiments, AG-120 in DMSO (3 μM), Glutaminase inhibitor (1 μM), or 2-DG (10 mM) was added to the cell culture media before plating the cells. For all conditions and cell lines, the cell culture supernatant was collected after 48 hours of incubation at 37° C. 10 μL of cell culture media was mixed with 90 μL of purified D2HGlo (5 μM), and the FRET ratios were collected. D-2-HG levels were estimated by using the titration curve as a calibration curve and converting the FRET ratios of unknowns into concentrations. Biological Fluids: Urine and serum were collected from three healthy donors while aCSF was purchased (Tocris Bioscience). Biological fluids were spiked with D-2-HG at concentrations ranging from 10 nM to 1 mM. Fluorescence was collected at 480 nm or 485 nm and 531 nm, and the FRET ratio was determined. Binding constants were determined the same as earlier.

Tumor Lysates: Advocate Aurora Research Institute, LLC, Milwaukee, WI, generously provided Archived Tumor Samples with known IDH1 mutational status (Aurora IRB #14-79), which was further confirmed using CPNA-LAMP. Tumor lysates were created by homogenizing approximately 0.1 gram of tumor in 500 μL of physiological saline. A portion of the archived samples were identified as post-processing cells (samples 10, 12, 15, 16, and 18) and were analyzed directly. Prior to measurement with FRET, the samples were thawed and centrifuged at 16,000×G for 4 minutes. For quantification of D-2-HG, twenty tumor-derived lysates were mixed (in triplicate) with purified 5 μM D2HGlo in a 96-well plate, and the FRET ratio was recorded using a Synergy Multi-Mode Plate Reader using the same settings as described in the in vitro characterization of D2HGlo. The average [D-2-HG] for each tumor lysate was determined using a calibration curve of [D-2-HG] vs. FRET ratio with the following equation:

$$FRET ratio = ( 983540.33 \times {[ D - 2 - HG ]}^{0.9078} + 5.8039281 ) / ( 358522.33 \times {[ D - 2 - HG ]}^{0.9078} + 3.4788086 ) .$$

Availability of data and materials: The DNA and protein sequences for D2HGlo are available in GenBank (Banklt2901380 BSeq #1 PQ777100), and in the current application. Raw data files and raw fluorescence images/videos are available upon request.

### Statistics and Reproducibility

At least three independent experiments were performed for each assay. Data is shown as either representative experiments (binding curves) or individual data points (bar graphs). Statistical analysis was performed using GraphPad Prism 10.1.2.

Abbreviations

| | |
|---|---|
| Acute myeloid leukemia | AML |
| Apparent binding affinity | K_{d}′ |
| Artificial cerebral spinal fluid | aCSF |
| circularly permuted variant of Venus | cpV173 |
| Classification of Tumors of the Central | CNS5 |
| Nervous System | |
| D-2-HG dehydrogenase regulator | DhdR |
| D-2-Hydroxyglutarate | D-2-HG |
| Dynamic range | Rₘₐₓ/Rₘᵢₙ |
| Enhanced Cyan Fluorescent Protein | ECFP |
| Fluorescence resonance energy transfer | FRET |
| gas chromatography-mass spectrometry | GC-MS |
| Glioblastoma | GBM |
| Global-CpG Island methylation phenotype | G-CIMP |
| Immunohistochemistry | IHC |
| Isocitrate dehydrogenase | IDH |
| L-2-Hydroxyglutarate | L-2-HG |
| Lactate dehydrogenase | LDH |
| liquid chromatography-tandem mass | LC-MS/MS |
| spectrometry | |
| Magnetic resonance spectroscopy | MRS |
| Malate dehydrogenase | MDH |
| Maximum FRET ratio | Rₘₐₓ |
| Minimum FRET ratio | Rₘᵢₙ |
| Poly(ADP)-ribose polymerase | PARP |
| Wildtype | WT |
| World Health Organization | WHO |

PCR Primers

| | | |
|---|---|---|
| Parent sensor forward | SEQ ID | CATCATGCATGCATATGCTGAGCAAGAGCCTGACC |
| primer | NO: 24 | |
| Parent sensor reverse | SEQ ID | ATGATGGAGCTCTGATGTCTGCCTTGCGGCC |
| primer | NO: 25 | |
| Variant 1 forward | SEQ ID | CATCATGCATGCATCTGAGCAAGAGCCTGACCTTGAC |
| primer | NO: 26 | |
| Variant 1 reverse | SEQ ID | ATGATGGAGCTCTGTCTCTGCCTTGCGGCCG |
| primer | NO: 27 | |
| Variant 2 forward | SEQ ID | CATCATGCATGCATAGCAAGAGCCTGACCTTGACCG |
| primer | NO: 28 | |
| Variant 2 reverse | SEQ ID | ATGATGGAGCTCCTGCCTTGCGGCCG |
| primer | NO: 29 | |
| Variant 3 forward | SEQ ID | CATCATGCATGCATAAGAGCCTGACCTTGACCGAAC |
| primer | NO: 30 | |
| Variant 3 reverse | SEQ ID | ATGATGGAGCTCCCTTGCGGCCGGAGAGAG |
| primer | NO: 31 | |
| T52A forward primer | SEQ ID | CCGCGAGGCCGCCGAGCGCC |
| | NO: 32 | |
| T52A reverse primer | SEQ ID | ATGACCGCGGCGCTCACACCG |
| | NO: 33 | |
| S69A forward primer | SEQ ID | GCCAGGGCTCGGGCGCCCGTGGTGGTGTCCCGC |
| | NO: 34 | |
| S69A reverse primer | SEQ ID | GGCTCTGGATCAGCCCCTGGGCGC |
| | NO: 35 | |
| S69T forward primer | SEQ ID | GCCAGGGCTCGGGCACCGTGGTGGTGTCCC |
| | NO: 36 | |
| S69T reverse primer | SEQ ID | GGCTCTGGATCAGCCCCTGGGCGC |
| | NO: 37 | |
| Cyto-D2HGlo forward | SEQ ID | CATCATGGATCCCATGGTGAGCAAGGGCGAGG |
| primer | NO: 38 | |
| Cyto-D2HGlo reverse | SEQ ID | GCCAAAACAGCCAAGCTTCGAATTC |
| primer | NO: 39 | |
| Nuc-D2HGlo forward | SEQ ID | CATCATGGTACCATGCCTAAAAAAAAACGTAAAGTT |
| primer | NO: 40 | GAAGATGCTTCGGATCCCATGGTGAGCAAGGGCGAGG |
| Nuc-D2HGlo reverse | SEQ ID | GCCAAAACAGCCAAGCTTCGAATTC |
| primer | NO: 41 | |
| Mito-D2HGlo forward | SEQ ID | CATCATGGATCCCATGGTGAGCAAGGGCGAGG |
| primer | NO: 42 | |
| Mito-D2HGlo reverse | SEQ ID | GCCAAAACAGCCAAGCTTCGAATTC |
| primer | NO: 43 | |

Supplemental Table 1. Primers used in molecular cloning. The forward and reverse primers for generating the initial DhdR-based sensor construct, three D2HGlo linker variants, three DhdR binding pocket mutants and the three cellular D2HGlo constructs.

### References

Beiko, J. et al. IDH1 mutant malignant astrocytomas are more amenable to surgical resection and have a survival benefit associated with maximal surgical resection. Neuro Oncol 16, 81-91 (2014). https://doi.org/10.1093/neuonc/not159
Bunse, L. et al. Suppression of antitumor T cell immunity by the oncometabolite (R)-2-hydroxyglutarate. Nature Medicine 24, 1192-1203 (2018). https://doi.org/10.1038/s41591-018-0095-6
Cahill, D. P. Extent of resection of glioblastoma: A critical evaluation in the molecular era. Neurosurg Clin N Am 32, 23-29 (2021). https://doi.org/10.1016/j.nec.2020.09.006
Capper, D. et al. 2-Hydroxyglutarate concentration in serum from patients with gliomas does not correlate with IDH1/2 mutation status or tumor size. Int J Cancer 131, 766-768 (2012). https://doi.org/10.1002/ijc.26425
Ceccarelli, M. et al. Molecular profiling reveals biologically discrete subsets and pathways of progression in diffuse glioma. Cell 164, 550-563 (2016). https://doi.org/10.1016/j.cell.2015.12.028
Choate, K. A. et al. Rapid extraction-free detection of the R132H isocitrate dehydrogenase mutation in glioma using colorimetric peptide nucleic acid-loop mediated isothermal amplification (CPNA-LAMP). PLOS One 18, e0291666 (2023). https://doi.org/10.1371/journal.pone.0291666
Choate, K. A. et al. Rapid IDH1-R132 genotyping panel utilizing locked nucleic acid loop-mediated isothermal amplification (LNA-LAMP). Biology Methods and Protocols (2024). https://doi.org/10.1093/biomethods/bpae012
Choi, C. et al. 2-hydroxyglutarate detection by magnetic resonance spectroscopy in IDH-mutated patients with gliomas. Nat Med 18, 624-629 (2012). https://doi.org/10.1038/nm.2682
Chowdhury, R. et al. The oncometabolite 2-hydroxyglutarate inhibits histone lysine demethylases. EMBO Rep 12, 463-469 (2011). https://doi.org/10.1038/embor.2011.43
Cui, D. et al. R132H mutation in IDH1 gene reduces proliferation, cell survival and invasion of human glioma by downregulating Wnt/β-catenin signaling. Int J Biochem Cell Biol 73, 72-81 (2016). https://doi.org/10.1016/j.biocel.2016.02.007
Dang, L. et al. Cancer-associated IDH1 mutations produce 2-hydroxyglutarate. Nature 462, 739-744 (2009). https://doi.org/10.1038/nature08617
Delahousse, J. et al. Circulating oncometabolite D-2-hydroxyglutarate enantiomer is a surrogate marker of isocitrate dehydrogenase—mutated intrahepatic cholangiocarcinomas. European Journal of Cancer 90, 83-91 (2018). https://doi.org/https://doi.org/10.1016/j.ejca.2017.11.024
Ewald, J. C. et al. Engineering genetically encoded nanosensors for real-time in vivo measurements of citrate concentrations. PLOS One 6, e28245 (2011). https://doi.org/10.1371/journal.pone.0028245
Fan, B. et al. Clinical pharmacokinetics and pharmacodynamics of ivosidenib in patients with advanced hematologic malignancies with an IDH1 mutation. Cancer Chemother Pharmacol 85, 959-968 (2020). https://doi.org/10.1007/s00280-020-04064-6
Fan, B. et al. Clinical pharmacokinetics and pharmacodynamics of ivosidenib, an oral, targeted inhibitor of mutant IDH1, in patients with advanced solid tumors. Invest New Drugs 38, 433-444 (2020). https://doi.org/10.1007/s10637-019-00771-x
Fan, B. et al. Pharmacokinetic/pharmacodynamic evaluation of Ivosidenib or Enasidenib combined with intensive induction and consolidation chemotherapy in patients with newly diagnosed IDH1/2-mutant acute myeloid leukemia. Clin Pharmacol Drug Dev 11, 429-441 (2022). https://doi.org/10.1002/cpdd.1067
Fathi, A. T. et al. Elevation of urinary 2-Hydroxyglutarate in IDH-Mutant glioma. Oncologist 21, 214-219 (2016). https://doi.org/10.1634/theoncologist.2015-0342
Fernandez-Galan, E. et al. Validation of a routine gas chromatography mass spectrometry method for 2-hydroxyglutarate quantification in human serum as a screening tool for detection of idh mutations. J Chromatogr B Analyt Technol Biomed Life Sci 1083, 28-34 (2018). https://doi.org/10.1016/j.jchromb.2018.02.038
Fujita, Y. et al. IDH1 p.R132H ctDNA and D-2-hydroxyglutarate as CSF biomarkers in patients with IDH-mutant gliomas. J Neurooncol 159, 261-270 (2022). https://doi.org/10.1007/s11060-022-04060-1
Hartmann, C. et al. Patients with IDH1 wild type anaplastic astrocytomas exhibit worse prognosis than IDH1-mutated glioblastomas, and IDH1 mutation status accounts for the unfavorable prognostic effect of higher age: implications for classification of gliomas. Acta Neuropathol 120, 707-718 (2010). https://doi.org/10.1007/s00401-010-0781-z
Intlekofer, A. M. et al. L-2-Hydroxyglutarate production arises from noncanonical enzyme function at acidic pH. Nat Chem Biol 13, 494-500 (2017). https://doi.org/10.1038/nchembio.2307
Jakola, A. S. et al. The impact of resection in IDH-mutant WHO grade 2 gliomas: a retrospective population-based parallel cohort study. J Neurosurg, 1-8 (2022). https://doi.org/10.3171/2022.1.JNS212514
Kalinina, J. et al. Selective detection of the D-enantiomer of 2-Hydroxyglutarate in the CSF of glioma patients with mutated isocitrate dehydrogenase. Clin Cancer Res 22, 6256-6265 (2016). https://doi.org/10.1158/1078-0432.CCR-15-2965
Kusi, M. et al. 2-Hydroxyglutarate destabilizes chromatin regulatory landscape and lineage fidelity to promote cellular heterogeneity. Cell Rep 38, 110220 (2022). https://doi.org/10.1016/j.celrep.2021.110220
Lee, C. L. et al. Circulating oncometabolite 2-hydroxyglutarate as a potential biomarker for isocitrate dehydrogenase (IDH1/2) mutant cholangiocarcinoma. Mol Cancer Ther 23, 394-399 (2024). https://doi.org/10.1158/1535-7163.Mct-23-0460
Lombardi, G. et al. Diagnostic value of plasma and urinary 2-hydroxyglutarate to identify patients with isocitrate dehydrogenase-mutated glioma. Oncologist 20, 562-567 (2015). https://doi.org/10.1634/theoncologist.2014-0266
Louis, D. N. et al. The 2021 WHO classification of tumors of the central nervous system: a summary. Neuro Oncol 23, 1231-1251 (2021). https://doi.org/10.1093/neuonc/noab106
Luddecke, J. et al. P (II) Protein-derived FRET sensors for quantification and live-cell imaging of 2-Oxoglutarate. Sci Rep 7, 1437 (2017). https://doi.org/10.1038/s41598-017-01440-w
Malta, T. M. et al. Glioma CpG island methylator phenotype (G-CIMP): biological and clinical implications. Neuro Oncol 20, 608-620 (2018). https://doi.org/10.1093/neuonc/nox183
Mellinghoff, I. K. et al. Vorasidenib in IDH1- or IDH2-mutant low-grade glioma. N Engl J Med 389, 589-601 (2023). https://doi.org/10.1056/NEJMoa2304194
Molinaro, A. M. et al. Association of maximal extent of resection of contrast-enhanced and non-contrast-enhanced tumor with survival within molecular subgroups of patients with newly diagnosed glioblastoma. JAMA Oncol 6, 495-503 (2020). https://doi.org/10.1001/jamaoncol.2019.6143
Nakagawa, M. et al. Clinical usefulness of 2-hydroxyglutarate as a biomarker in IDH-mutant chondrosarcoma. J Bone Oncol 34, 100430 (2022). https://doi.org/10.1016/j.jbo.2022.100430
Philip, B. et al. Mutant IDH1 promotes glioma formation in vivo. Cell Rep 23, 1553-1564 (2018). https://doi.org/10.1016/j.celrep.2018.03.133
Pomorski, A. et al. Method for accurate determination of dissociation constants of optical ratiometric systems: chemical probes, genetically encoded sensors, and interacting molecules. Anal Chem 85, 11479-11486 (2013). https://doi.org/10.1021/ac402637h
Popovici-Muller, J. et al. Discovery of AG-120 (Ivosidenib): A first-in-class mutant IDH1 inhibitor for the treatment of IDH1 mutant cancers. ACS Med Chem Lett 9, 300-305 (2018). https://doi.org/10.1021/acsmedchemlett.7b00421
Pratt, E. P. S. et al. Regulation of CAMP accumulation and activity by distinct phosphodiesterase subtypes in INS-1 cells and human pancreatic B-cells. PLOS One 14, e0215188 (2019). https://doi.org/10.1371/journal.pone.0215188
Pratt, E. P. S. et al. Tools and techniques for illuminating the cell biology of zinc. Biochim Biophys Acta Mol Cell Res 1868, 118865 (2021). https://doi.org/10.1016/j.bbamcr.2020.118865
Pusch, S. et al. D-2-Hydroxyglutarate producing neo-enzymatic activity inversely correlates with frequency of the type of isocitrate dehydrogenase 1 mutations found in glioma. Acta Neuropathol Commun 2, 19 (2014). https://doi.org/10.1186/2051-5960-2-19
Raimondi, V., Ciotti, G., Gottardi, M. & Ciccarese, F. 2-Hydroxyglutarate in acute myeloid leukemia: A Journey from Pathogenesis to Therapies. Biomedicines 10 (2022). https://doi.org/10.3390/biomedicines10061359
Rzem, R. et al. L-2-hydroxyglutaric aciduria, a defect of metabolite repair. J Inherit Metab Dis 30, 681-689 (2007). https://doi.org/10.1007/s10545-007-0487-0
Sim, H. W. et al. Tissue 2-Hydroxyglutarate as a biomarker for isocitrate dehydrogenase mutations in gliomas. Clin Cancer Res 25, 3366-3373 (2019). https://doi.org/10.1158/1078-0432.Ccr-18-3205
Strain, S. K., et al. Enantioseparation and detection of (R)-2-Hydroxyglutarate and(S)-2-Hydroxyglutarate by chiral gas chromatography-triple quadrupole mass spectrometry. Metabolomics, 89-100 (2021). https://doi.org/10.1007/978-1-0716-0864-7_8
Struys, E. et al. Measurement of urinary D-and L-2-hydroxyglutarate enantiomers by stable-isotope-dilution liquid chromatography-tandem mass spectrometry after derivatization with diacetyl-L-tartaric anhydride. Clin Chem 50, 1391-1395 (2004). https://doi.org/10.1373/clinchem.2004.033399
Tuna, G. et al. Minimally invasive detection of IDH1 mutation with cell-free circulating tumor DNA and D-2-Hydroxyglutarate, D/L-2-Hydroxyglutarate ratio in gliomas. J Neuropathol Exp Neurol 81, 502-510 (2022). https://doi.org/10.1093/jnen/nlac036
Turcan, S. et al. IDH1 mutation is sufficient to establish the glioma hypermethylator phenotype. Nature 483, 479-483 (2012). https://doi.org/10.1038/nature10866
Voelxen, N. F. et al. Quantitative imaging of D-2-Hydroxyglutarate in selected histological tissue areas by a novel bioluminescence technique. Front Oncol 6, 46 (2016). https://doi.org/10.3389/fonc.2016.00046
Wang, J. H. et al. Prognostic significance of 2-hydroxyglutarate levels in acute myeloid leukemia in China. Proc Natl Acad Sci U S A 110, 17017-17022 (2013). https://doi.org/10.1073/pnas.1315558110
Xiao, D. et al. A D-2-hydroxyglutarate biosensor based on specific transcriptional regulator DhdR. Nat Commun 12, 7108 (2021). https://doi.org/10.1038/s41467-021-27357-7
Xiao, D. et al. A Förster resonance energy transfer-based D-2-hydroxyglutarate biosensor. Sensors and Actuators B: Chemical 385, 133681 (2023). https://doi.org/https://doi.org/10.1016/j.snb.2023.133681
Yang, Z. et al. 2-HG inhibits necroptosis by stimulating DNMT1-dependent hypermethylation of the RIP3 promoter. Cell Rep 19, 1846-1857 (2017). https://doi.org/10.1016/j.celrep.2017.05.012
Ye, D., Guan, K. L. & Xiong, Y. Metabolism, activity, and targeting of D-and L-2-Hydroxyglutarates. Trends Cancer 4, 151-165 (2018). https://doi.org/10.1016/j.trecan.2017.12.005
Zhang, C. et al. Serum D-2-hydroxyglutarate and the ratio of D-2HG/L-2HG predict IDH mutation in acute myeloid leukemia. EJHaem 4, 723-727 (2023). https://doi.org/10.1002/jha2.723
Shankar, G. M. et al. Genotype-targeted local therapy of glioma. Proc Natl Acad Sci U S A 115, E8388-e8394 (2018). https://doi.org/10.1073/pnas.1805751115
Xue, H. et al. Application of Intraoperative Rapid Molecular Diagnosis in Precision Surgery for Glioma: Mimic the World Health Organization CNS5 Integrated Diagnosis. Neurosurgery 92, 762-771 (2023). https://doi.org/10.1227/neu.0000000000002260

1. A construct for detection of d-2-hydroxyglutarate (d-2-HG), comprising:
a first fluorescent reporter having an emission peak which is less than 490 nm;
a second fluorescent reporter having an excitation peak which is less than 550 nm; and
a DhdR transcription factor, or variant thereof,
wherein the first fluorescent reporter is linked at the N-terminus of the DhdR transcription factor, or variant thereof, and the second fluorescent reporter is linked at the C-terminus of the DhdR transcription factor, or variant thereof, or wherein the first fluorescent reporter is linked at the C-terminus of the DhdR transcription factor, or variant thereof, and the second fluorescent reporter is linked at the N-terminus of the DhdR transcription factor, or variant thereof, and
wherein the variant of the DhdR transcription factor comprises a truncated DhdR transcription factor for binding d-2-HG, in which up to 5 amino acid residues are truncated from the N-terminus of DhdR corresponding to the terminal N-terminus amino acid residues of DhdR, and/or up to 5 amino acid residues are truncated from the C-terminus of DhdR corresponding to the terminal C-terminus amino acid residues of DhdR.

2. A construct according to claim 1, wherein the first fluorescent reporter is linked at the N-terminus of the DhdR transcription factor, or variant thereof, by a first linker which comprises at least one amino acid residue, and the second fluorescent reporter is linked at the C-terminus of the DhdR transcription factor, or variant thereof, by a second linker which comprises at least one amino acid residue, or wherein the first fluorescent reporter is linked at the C-terminus of the DhdR transcription factor, or variant thereof, by a first linker which comprises at least one amino acid residue, and the second fluorescent reporter is linked at the N-terminus of the DhdR transcription factor, or variant thereof, by a second linker which comprises at least one amino acid residue.

3. A construct according to claim 1, wherein the first and the second fluorescent reporter comprise first and second fluorescent proteins, respectively.

4. A construct according to claim 3, wherein the first fluorescent protein is enhanced cyan fluorescent protein (ECFP), and the second fluorescent protein is cpVenus173.

5. A construct according to claim 1, in which two amino acid residues are truncated from the N-terminus of the truncated DhdR transcription factor, corresponding to the two terminal N-terminus amino acid residues of DhdR, and two amino acid residues are truncated from the C-terminus of the truncated DhdR transcription factor, corresponding to the two terminal C-terminus amino acid residues of DhdR transcription factor.

6. A construct according to claim 1, in which the N-terminus amino acid residues of the truncated DhdR transcription factor are the amino acid residues of SEQ ID NO:18, wherein the amino acid residues are present in the order recited in SEQ ID NO:18, beginning at the terminal N-terminus amino acid residue, or an amino acid sequence that has at least 75% amino acid identity along its entire length with the sequence of SEQ ID NO:18, and the C-terminus amino acid residues of the truncated DhdR transcription factor are the amino acid residues of SEQ ID NO:19, wherein the amino acid residues are present in the order recited in SEQ ID NO:19, beginning at the terminal C-terminus amino acid residue, or an amino acid sequence that has at least 75% amino acid identity along its entire length with the sequence of SEQ ID NO:19.

7. A construct according to claim 1, wherein the truncated DhdR transcription factor comprises an amino acid sequence of SEQ ID NO:2 (Variant 2 truncated DhdR).

8. A construct according to claim 1, which comprises an amino acid sequence of SEQ ID NO: 4.

9. A construct according to claim 1, wherein the first fluorescent reporter has an excitation peak which is less than 480 nm and/or the second fluorescent reporter has an emission peak which is more than 490 nm.

10. A nucleic acid molecule encoding a construct according to claim 1, or an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with the nucleic acid molecule over its entire length, or the complement thereof.

11. A vector comprising a nucleic acid molecule according to claim 10.

12. A vector according to claim 11, which further comprises a promoter operably linked to the nucleic acid, preferably wherein the promoter is for expression of a polypeptide encoded by the nucleic acid in a protein expression cell line.

13. A vector according to claim 12, wherein the promoter is for expression of a polypeptide encoded by the nucleic acid in mammalian cells or bacterial cells.

14. An cell comprising a vector according to claim 11.

15. A pharmaceutical composition comprising a construct according to claim 1, and a pharmaceutically acceptable carrier, excipient, or diluent.

16. A pharmaceutical composition comprising a nucleic acid molecule according to claim 9, and a pharmaceutically acceptable carrier, excipient, or diluent.

17. A pharmaceutical composition comprising a vector according to claim 11, and a pharmaceutically acceptable carrier, excipient, or diluent.

18. A method for determining the IDH1/2 mutational status of a biological sample obtained from a subject, wherein the method comprises detecting for D-2-HG in the sample using a construct according to claim 1.

19. A method according to claim 18, wherein detecting for D-2-HG in the sample comprises determining the relative difference in fluorescence intensities between the first fluorescent reporter and the second fluorescent reporter, or between the second fluorescent reporter and the first fluorescent reporter.

20. A method according to claim 18, wherein determining the IDH1/2 mutational status of a biological sample obtained from a subject is performed before, with, or after treatment with a medicament.

21. A method according to claim 20, wherein the medicament is an IDH1 inhibitor.

22. A method for monitoring a change in D-2-HG levels in a subject, which comprises:
(i) obtaining a biological sample from a subject;
(ii) detecting for D-2-HG in the sample by determining the relative difference in fluorescence intensities between the first fluorescent reporter and the second fluorescent reporter, or between the second fluorescent reporter and the first fluorescent reporter, in the biological sample using a construct according to any of claims 1 to 153;
(iii) repeating steps (i) and (ii) after a period of time; and
(iv) determining whether the relative difference in fluorescence intensities between the first fluorescent reporter and the second fluorescent reporter, or between the second fluorescent reporter and the first fluorescent reporter, has changed.

23. A method according to claim 22, wherein monitoring a change in D-2-HG levels in a subject is performed before, with, or after treatment with a medicament.

24. A method according to claim 23, wherein the medicament is an IDH1 inhibitor.

25. A method of analysing D-2-HG in a biological sample obtained from a subject, comprising detecting for D-2-HG in the cytoplasm or subcellular compartments of a cell in the biological sample using a construct which comprises a DhdR transcription factor linked to a first fluorescent reporter and a second fluorescent reporter.

26. A method according to claim 25, wherein the construct comprises a construct according to claim 1.

27. A method according to claim 25, wherein detecting for D-2-HG in the cytoplasm or subcellular compartments of the cell comprises determining the relative difference in fluorescence intensities between the first fluorescent reporter and the second fluorescent reporter, or between the second fluorescent reporter and the first fluorescent reporter.

28. A method according to claim 25, wherein the subcellular location is the mitochondria, cytoplasm, or the nucleus.


SEQUENCE LISTING (43 sequences)

SEQ ID NO: 1 - protein, 238 aa, Achromobacter denitrificans
MLSKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQTS

SEQ ID NO: 2 - protein, 234 aa, synthetic construct
SKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQ

SEQ ID NO: 3 - DNA, 702 bp, synthetic construct
  source 1..702: mol_type = other DNA
agcaagagcctgaccttgaccgaacaggtcgcccgccagatcgcgggcgacatcgccgaaggcgtccattccgtgggcgccaagctgccgcccggccgtgtcctggcggagcagtacggtgtgagcgccgcggtcatccgcgaggccaccgagcgcctgcgcgcccaggggctgatccagagccgccagggctcgggcagcgtggtggtgtcccgcaccggtgctcagggcttccaggtttccgccggcctcgacgatcgcgagcagctggccagcgtctacgaattgcggatggaactggaaggcggcgcggccgccctggcggcgaggcgccgcaacgccaccgaccttgcggccatggccgaggccctggccgcgctggaagcgaacctggaccatccggaacagggcgtcgagcacgacatcgccttccacgtcgccatcgccgccgccacgcacaaccgttattaccaggacctgctgcagtacctgaacctgcagctgcgcctggccgtcagcaccgcgcgcaccaacagccgccgtcaggagggcctgaccgcggtggtgcaccaggaacacgtggccgtctacgacgccatcctcgcgggcgatcccgaccgcgcccgactggcggcgacccgccacttgcagcaggcggccagccgcctgcgtctcgatctcctctctccggccgcaaggcag

SEQ ID NO: 4 - protein, 712 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMHSKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQELMDGGVQLADHYQQNTPIGDGPVLLPDNHYLSYQSALSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 5 - DNA, 2136 bp, synthetic construct
  source 1..2136: mol_type = other DNA
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgcatagcaagagcctgaccttgaccgaacaggtcgcccgccagatcgcgggcgacatcgccgaaggcgtccattccgtgggcgccaagctgccgcccggccgtgtcctggcggagcagtacggtgtgagcgccgcggtcatccgcgaggccaccgagcgcctgcgcgcccaggggctgatccagagccgccagggctcgggcagcgtggtggtgtcccgcaccggtgctcagggcttccaggtttccgccggcctcgacgatcgcgagcagctggccagcgtctacgaattgcggatggaactggaaggcggcgcggccgccctggcggcgaggcgccgcaacgccaccgaccttgcggccatggccgaggccctggccgcgctggaagcgaacctggaccatccggaacagggcgtcgagcacgacatcgccttccacgtcgccatcgccgccgccacgcacaaccgttattaccaggacctgctgcagtacctgaacctgcagctgcgcctggccgtcagcaccgcgcgcaccaacagccgccgtcaggagggcctgaccgcggtggtgcaccaggaacacgtggccgtctacgacgccatcctcgcgggcgatcccgaccgcgcccgactggcggcgacccgccacttgcagcaggcggccagccgcctgcgtctcgatctcctctctccggccgcaaggcaggagctcatggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggtggcagcggtggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgag

SEQ ID NO: 6 - protein, 228 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAA

SEQ ID NO: 7 - DNA, 684 bp, synthetic construct
  source 1..684: mol_type = other DNA
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcc

SEQ ID NO: 8 - protein, 245 aa, synthetic construct
MDGGVQLADHYQQNTPIGDGPVLLPDNHYLSYQSALSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 9 - DNA, 735 bp, synthetic construct
  source 1..735: mol_type = other DNA
atggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggtggcagcggtggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgag

SEQ ID NO: 10-13 - skipped (4 entries)

SEQ ID NO: 14 - protein, 236 aa, synthetic construct
LSKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQT

SEQ ID NO: 15 - protein, 232 aa, synthetic construct
KSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAAR

SEQ ID NO: 16 - protein, 714 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMHLSKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARQTELMDGGVQLADHYQQNTPIGDGPVLLPDNHYLSYQSALSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 17 - protein, 710 aa, synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMHKSLTLTEQVARQIAGDIAEGVHSVGAKLPPGRVLAEQYGVSAAVIREATERLRAQGLIQSRQGSGSVVVSRTGAQGFQVSAGLDDREQLASVYELRMELEGGAAALAARRRNATDLAAMAEALAALEANLDHPEQGVEHDIAFHVAIAAATHNRYYQDLLQYLNLQLRLAVSTARTNSRRQEGLTAVVHQEHVAVYDAILAGDPDRARLAATRHLQQAASRLRLDLLSPAARELMDGGVQLADHYQQNTPIGDGPVLLPDNHYLSYQSALSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIE

SEQ ID NO: 18 - protein, 4 aa, synthetic construct
SKSL

SEQ ID NO: 19 - protein, 4 aa, synthetic construct
QRAA

SEQ ID NO: 20 - protein, 4 aa, synthetic construct
KSLT

SEQ ID NO: 21 - protein, 4 aa, synthetic construct
RAAP

SEQ ID NO: 22 - protein, 4 aa, synthetic construct
LSKS

SEQ ID NO: 23 - protein, 4 aa, synthetic construct
TQRA

SEQ ID NO: 24 - DNA, 35 bp, synthetic construct
  source 1..35: mol_type = other DNA
catcatgcatgcatatgctgagcaagagcctgacc

SEQ ID NO: 25 - DNA, 31 bp, synthetic construct
  source 1..31: mol_type = other DNA
atgatggagctctgatgtctgccttgcggcc

SEQ ID NO: 26 - DNA, 37 bp, synthetic construct
  source 1..37: mol_type = other DNA
catcatgcatgcatctgagcaagagcctgaccttgac

SEQ ID NO: 27 - DNA, 31 bp, synthetic construct
  source 1..31: mol_type = other DNA
atgatggagctctgtctctgccttgcggccg

SEQ ID NO: 28 - DNA, 36 bp, synthetic construct
  source 1..36: mol_type = other DNA
catcatgcatgcatagcaagagcctgaccttgaccg

SEQ ID NO: 29 - DNA, 26 bp, synthetic construct
  source 1..26: mol_type = other DNA
atgatggagctcctgccttgcggccg

SEQ ID NO: 30 - DNA, 36 bp, synthetic construct
  source 1..36: mol_type = other DNA
catcatgcatgcataagagcctgaccttgaccgaac

SEQ ID NO: 31 - DNA, 30 bp, synthetic construct
  source 1..30: mol_type = other DNA
atgatggagctcccttgcggccggagagag

SEQ ID NO: 32 - DNA, 20 bp, synthetic construct
  source 1..20: mol_type = other DNA
ccgcgaggccgccgagcgcc

SEQ ID NO: 33 - DNA, 21 bp, synthetic construct
  source 1..21: mol_type = other DNA
atgaccgcggcgctcacaccg

SEQ ID NO: 34 - DNA, 33 bp, synthetic construct
  source 1..33: mol_type = other DNA
gccagggctcgggcgcccgtggtggtgtcccgc

SEQ ID NO: 35 - DNA, 24 bp, synthetic construct
  source 1..24: mol_type = other DNA
ggctctggatcagcccctgggcgc

SEQ ID NO: 36 - DNA, 30 bp, synthetic construct
  source 1..30: mol_type = other DNA
gccagggctcgggcaccgtggtggtgtccc

SEQ ID NO: 37 - DNA, 24 bp, synthetic construct
  source 1..24: mol_type = other DNA
ggctctggatcagcccctgggcgc

SEQ ID NO: 38 - DNA, 32 bp, synthetic construct
  source 1..32: mol_type = other DNA
catcatggatcccatggtgagcaagggcgagg

SEQ ID NO: 39 - DNA, 25 bp, synthetic construct
  source 1..25: mol_type = other DNA
gccaaaacagccaagcttcgaattc

SEQ ID NO: 40 - DNA, 73 bp, synthetic construct
  source 1..73: mol_type = other DNA
catcatggtaccatgcctaaaaaaaaacgtaaagttgaagatgcttcggatcccatggtgagcaagggcgagg

SEQ ID NO: 41 - DNA, 25 bp, synthetic construct
  source 1..25: mol_type = other DNA
gccaaaacagccaagcttcgaattc

SEQ ID NO: 42 - DNA, 32 bp, synthetic construct; identical to SEQ ID NO: 38
  source 1..32: mol_type = other DNA

SEQ ID NO: 43 - DNA, 25 bp, synthetic construct
  source 1..25: mol_type = other DNA
gccaaaacagccaagcttcgaattc